(19) United States
(12) Patent Application Publication
(10) Pub. No.: US 2009/0137566 A1
(43) Pub. Date: May 28, 2009

(54) Substituted Piperdines as Renin Inhibitors

(76) Inventors: Takeru Ehara, Ibaraki (JP); Yuko Hitomi, Ibaraki (JP); Kazuhide Konishi, Ibaraki (JP); Kelichi Masuya, Bottmingen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/914,869

(22) PCT Filed: May 24, 2006
(86) PCT No.: PCT/EP2006/004941

§ 371 Date: Nov. 19, 2007

(30) Foreign Application Priority Data
May 26, 2005 (US) 0510810.5

(51) Int. Cl. A61K 31/5415; A61K 31/538; A61K 31/445; A61K 31/4545; A61K 31/454; C07D 417/02; C07D 413/02; C07D 405/02; C07D 409/02

(52) U.S. Cl. 514/224.2; 514/230.5; 514/323; 514/307; 514/314; 514/321; 514/318; 544/51; 544/105; 546/148; 546/167; 546/201; 546/193; 546/196; 546/225

(57) ABSTRACT

The invention relates to substituted 3,4- or higher substituted piperididine compounds, the use thereof for the preparation of a pharmaceutical formulation for the treatment of a disease that depends on activity of renin; the use of a compound of that class in the treatment of a disease that depends on activity of renin; these compounds for use in the diagnostic and therapeutic treatment of a warm-blooded animal, especially for the treatment of a disease (=disorder) that depends on activity of renin; pharmaceutical formulations or products comprising said compounds, and/or a method of treatment comprising administering said compounds, a method for the manufacture of said compounds, as well as novel intermediates, starting materials and/or partial steps for their synthesis. The compounds preferably have the formula I,

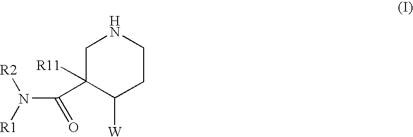

wherein the moieties R1, R2, R11 and W are as defined in the specification.

The invention relates to substituted 3,4- or higher substituted piperidine compounds, the use thereof for the preparation of a pharmaceutical formulation for the treatment of a disease that depends on activity of renin; the use of a compound of that class in the treatment of a disease that depends on activity of renin; these compounds for use in the diagnostic and therapeutic treatment of a warm-blooded animal, especially for the treatment of a disease (=disorder) that depends on activity of renin; pharmaceutical formulations or products comprising said compounds, and/or a method of treatment comprising administering said compounds, a method for the manufacture of said compounds, as well as novel intermediates, starting materials and/or partial steps for their synthesis.

The invention especially relates to a compound of the formula I,

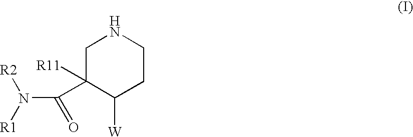

wherein
R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is unsubstituted or substituted polycyclic heterocyclyl or unsubstituted or substituted polycyclic aryl; and
R11 is hydrogen, hydroxy, halo, C₁-C₇-alkyl, halo-C₁-C₇-alkyl, cycloalkyl, halo-substituted cycloalkyl, C₁-C₇-alkoxy, halo-C₁-C₇-alkoxy or cyano,
or a (preferably pharmaceutically acceptable) salt thereof.

The compounds of the present invention exhibit inhibitory activity on the natural enzyme renin. Thus, compounds of formula I may be employed for the treatment (this term also including prophylaxis) of one or more disorders or diseases especially selected from the diseases given in detail below, especially as far as these diseases can be modulated (more especially beneficially influenced) by renin inhibition.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo. If not explicitly or implicitly stated otherwise, halo can also stand for more than one halogen substituent in moieties such as alkyl, alkanoyl and the like (e.g. in trifluoromethyl, trifluoroacetyl).

Unsubstituted or substituted alkyl is preferably C₁-C₂₀-alkyl, more preferably C₁-C₇-alkyl, that is straight-chained or branched (one or, if desired and possible, more times), and which is unsubstituted or substituted by one or more, e.g. up to three moieties independently selected from unsubstituted or substituted aryl as described below, especially phenyl or naphthyl each of which is unsubstituted or substituted as described below for unsubstituted or substituted aryl, unsubstituted or substituted heterocyclyl as described below, especially pyrrolyl, furanyl, thienyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydro-pyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl and benzo[1,2,5]oxadiazolyl each of which is unsubstituted or substituted as described below for unsubstituted or substituted heterocyclyl, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkylcarbonyl, C₁-C₇-alkoxycarbonyl, phenyl- or naphthyloxycarbonyl, phenyl- or naphthyl-C₁-C₇-alkoxycarbonyl, carbamoyl, N-mono- or N,N-di-(C₁-C₇-alkyl-, naphthyl- and/or phenyl-C₁-C₇-alkyl)-aminocarbonyl, cyano, C₁-C₇-alkenylene or -alkynylene, C₁-C₇-alkylenedioxy, sulfenyl (—S—OH), sulfinyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl (—S(O)₂OH), C₁-C₇-alkylsulfonyl (C₁-C₇-alkyl-SO₂—), phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl.

Unsubstituted or substituted alkenyl preferably has 2 to 20 carbon atoms and includes one or more double bonds, and is more preferably C₂-C₇-alkenyl that is unsubstituted or substituted as described above for unsubstituted or substituted alkyl. Examples are vinyl or allyl.

Unsubstituted or substituted alkynyl preferably has 2 to 20 carbon atoms and includes one or more triple bonds, and is more preferably C₂-C₇-alkynyl that is unsubstituted or substituted as described above for unsubstituted or substituted alkyl. An example is prop-2-ynyl.

Unsubstituted or substituted aryl preferably is a mono- or polycyclic, especially monocyclic, bicyclic or tricyclic aryl moiety with 6 to 22 carbon atoms, especially phenyl (very preferred), naphthyl (very preferred), indenyl, fluorenyl, acenapthylenyl, phenylenyl or phenanthryl, and is unsubstituted or substituted by one or more, especially one to three, moieties, preferably independently selected from the group consisting of a substituent of the formula —(C₀-C₇-alkylene)-(K)ₚ—(C₁-C₇-alkylene)-(L)_{q}—(C₀-C₇-alkylene)-H where C₀-alkylene means that a bond is present instead of bound alkylene, p and q, each independently of the other, are 0 or 1 and each of K and L, if present and independently of the others, is —O—, —NM—, —S—, —C(═O)—, —C(═S), —O—CO—, —CO—O—, —NM—CO—; —CO-NM—; —NM-SO₂—, —SO₂—NM; —NM-CO-NM—, —NM-CO—O—, —O—CO-NM-, —NM-SO₂—NM- wherein M is hydrogen or unsubstituted or substituted alkyl as defined below; especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N—) mono- or (N,N—) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkylcarbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N—) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl; from C₂-C₇-alkenyl, C₂-C₇-alkynyl, phenyl, naphthyl, heterocyclyl, especially as defined below for heterocyclyl, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydro-pyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein each phenyl, naphthyl or heterocyclyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano and wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyloxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N—) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N—) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N—) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl; where in each case where mentioned in this paragraph before phenyl, naphthyl or heterocyclyl is unsubstituted or substituted by one or more, especially up to three, moieties selected from the group consisting of C₁-C₇-alkyl, C₁-C₇-alkenyl, C₁-C₇-alkynyl, halo-C₁-C₇-alkyl, such as trifluoromethyl, halo, especially fluoro, chloro, bromo or iodo, hydroxy, C₁-C₇-alkoxy, phenyloxy, naphthyloxy, phenyl- or naphthyl-C₁-C₇-alkoxy, C₁-C₇-alkanoyloxy, phenyl- or naphthyl-C₁-C₇-alkanoyloxy, amino, mono- or di-(C₁-C₇-alkyl, phenyl, naphthyl, phenyl-C₁-C₇-alkyl, naphthyl-C₁-C₇-alkyl, C₁-C₇-alkanoyl and/or phenyl- or naphthyl-C₁-C₇-alkanoyl)-amino, carboxy, C₁-C₇-alkoxycarbonyl, phenoxycarbonyl, naphthyloxycarbonyl, phenyl-C₁-C₇-alkyloxycarbonyl, naphthyl-C₁-C₇-alkoxycarbonyl, carbamoyl, N-mono- or N,N-di-(C₁-C₇-alkyl, phenyl, naphthyl, phenyl-C₁-C₇-alkyl and/or naphthyl-C₁-C₇-alkyl)-aminocarbonyl, cyano, sulfo, sulfamoyl, N-mono- or N,N-di-(C₁-C₇-alkyl, phenyl, naphthyl, phenyl-C₁-C₇-alkyl and/or naphthyl-C₁-C₇-alkyl)-aminosulfonyl and nitro, or preferably, where preferred substituents are mentioned, by one or more of these mentioned substituents.

Especially preferred aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇-alkoxy, hydroxy-C₁-C₇-alkoxy, C₁-C₇-alkoxy-C₁-C₇-alkoxy; phenyl, naphthyl, phenyl-C₁-C₇-alkyl or naphthyl-C₁-C₇-alkyl wherein phenyl or naphthyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano; amino-C₁-C₇-alkoxy, N—C₁-C₇-alkanoylamino-C₁-C₇-alkoxy, carboxyl-C₁-C₇-alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano.

Unsubstituted or substituted polycyclic aryl is an aryl with two or more annealed rings, especially bi-, tri- or tetracyclic aryl, wherein at least one ring is unsaturated (=contains the highest possible number of conjugated double bonds between ring atoms). Preferably, polycyclic aryl, each of which is unsubstituted or substituted by one or more substituents independently selected from the substituents mentioned above for substituted aryl, is selected from the following group of moieties:

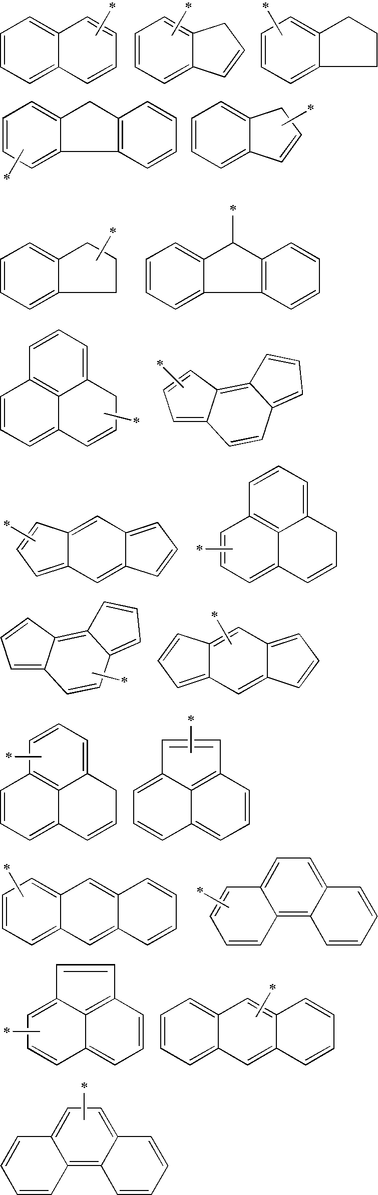

where the bond with asterisk marks the bond shown in formula I (and corresponding intermediates and starting materials) with which the respective moiety is bound to the rest of the molecule (so that any one H is present in such moiety may be replaced by that bond with the asterisk connecting the respective heterocyclyl moiety to the rest of the molecule, while one or more other H atoms may be replaced by a substituent if one or more substituents are present as just described);

Especially, unsubstituted or substituted polycyclic aryl is selected from the group consisting of naphthyl, fluorenyl and indenyl, each of which is unsubstituted or substituted by one or more, preferably up to three, moieties independently selected from those mentioned as substitutents for substituted aryl.

Unsubstituted or substituted heterocyclyl is a mono- or polycyclic, especially mono- or bicyclic, heterocyclic moiety with an unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen and sulfur (—S—, S(═O)— or S—(═O)₂—) which is unsubstituted or substituted by one or more, e.g. up to three, substitutents preferably independently selected from the substitutents mentioned above for aryl and from oxo (═O) and thioxo (═S). Preferably, unsubstituted or substituted heterocyclyl is selected from the following moieties:

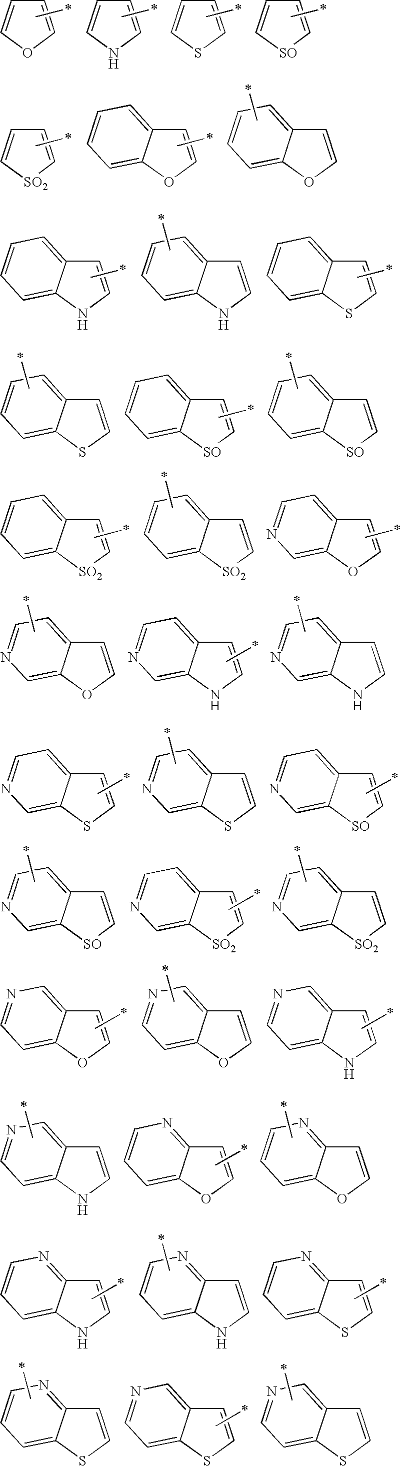

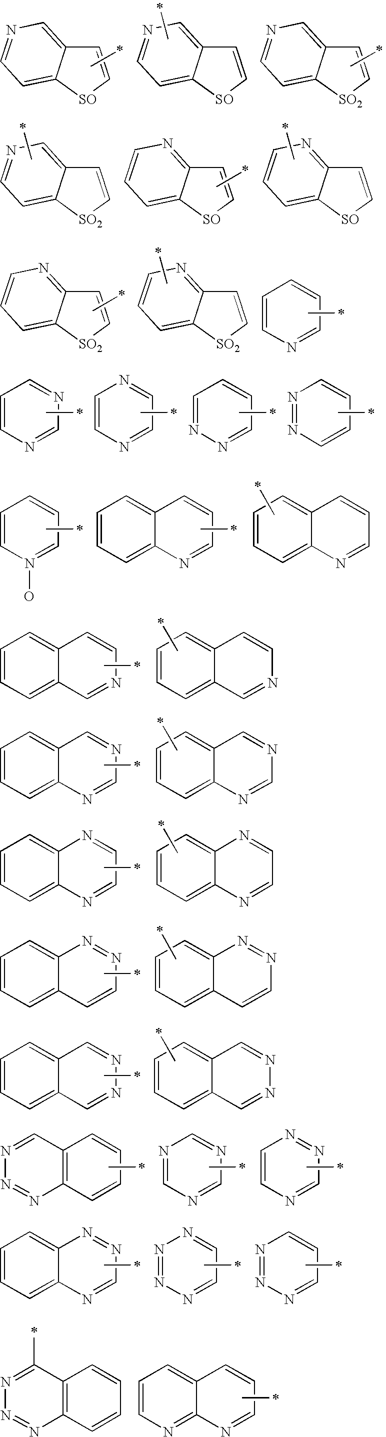

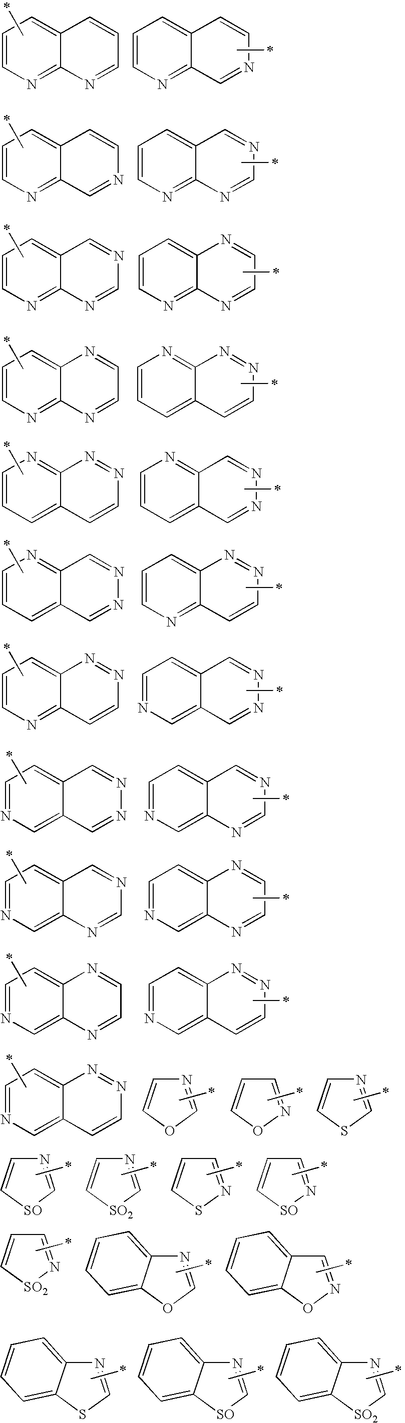

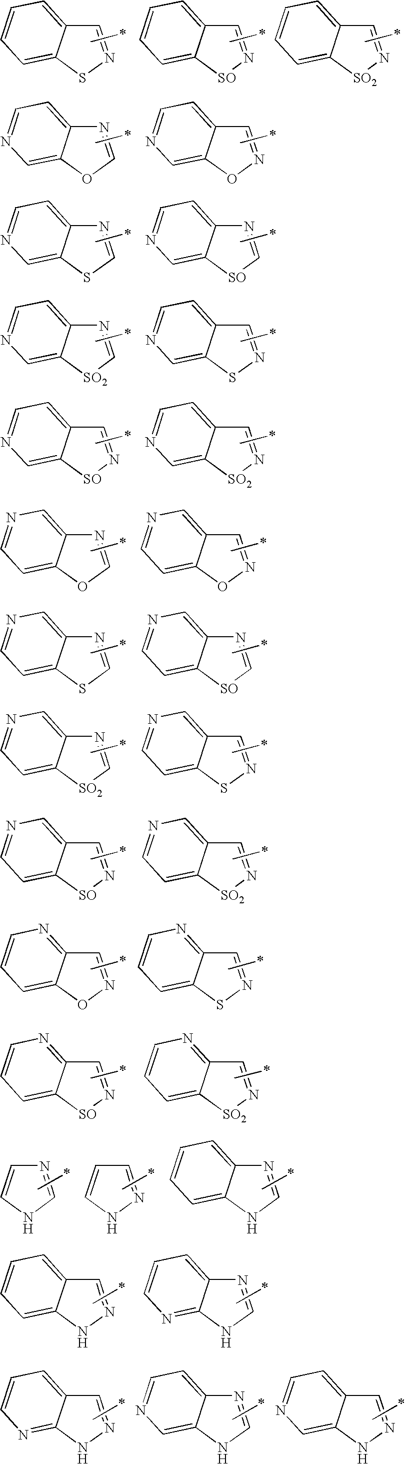

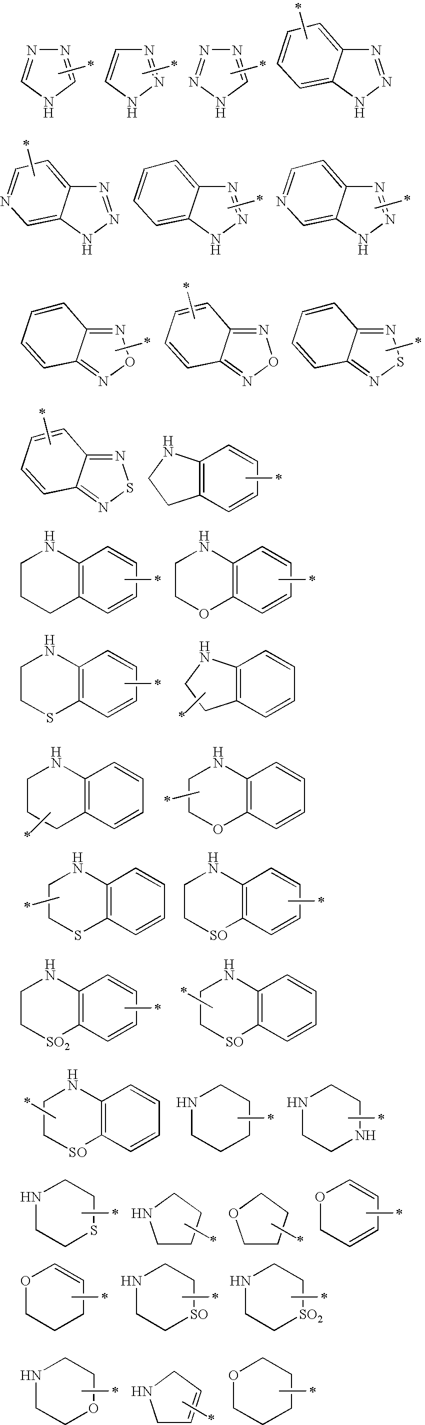

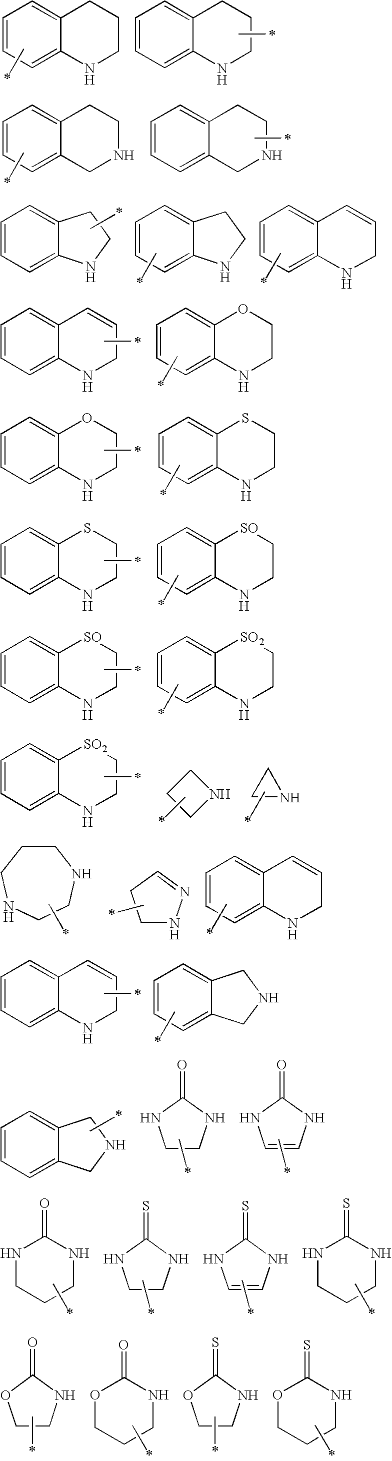

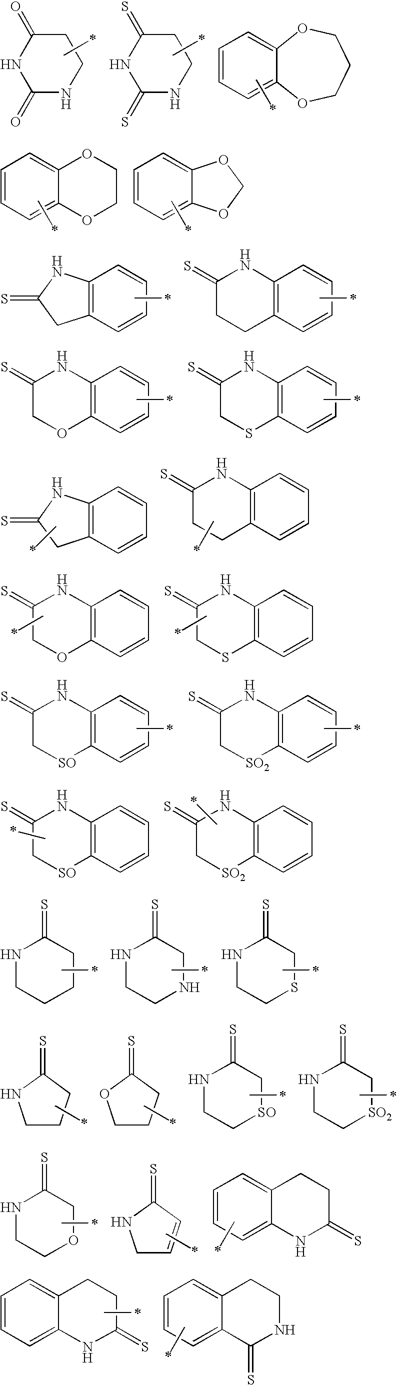

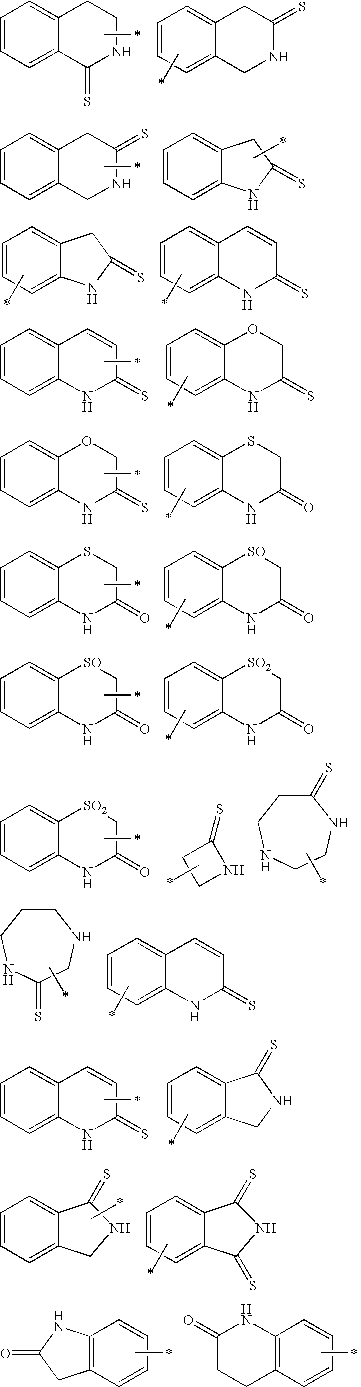

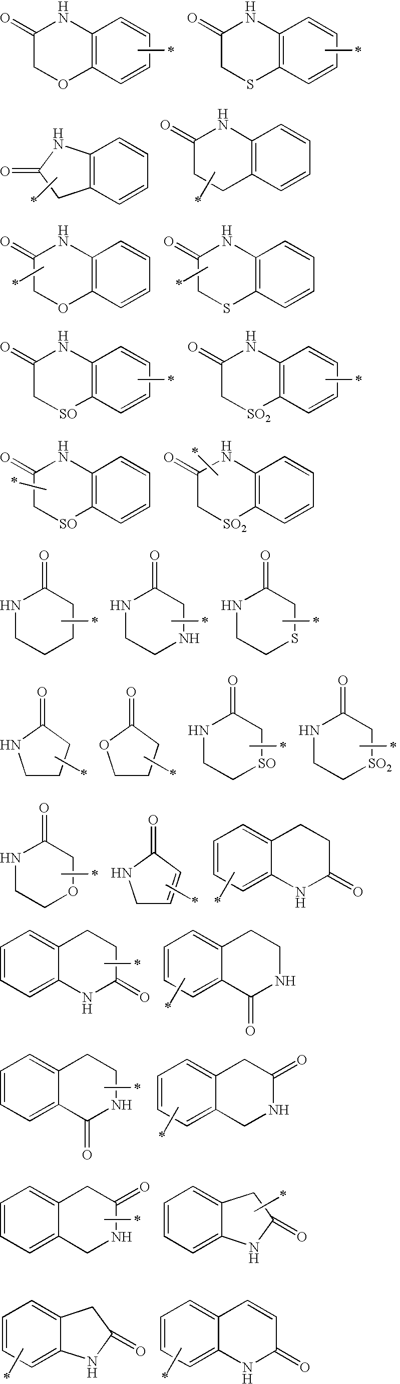

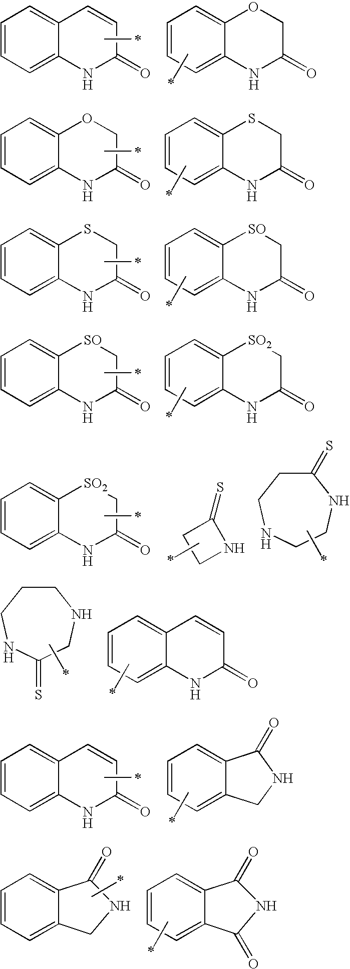

where the bond with asterisk marks the bond shown in formula I (and corresponding intermediates and starting materials) with which the respective moiety is bound to the rest of the molecule (so that any one H is present in such moiety may be replaced by that bond with the asterisk connecting the respective heterocyclyl moiety to the rest of the molecule, while one or more other H atoms may be replaced by a substituent if one or more substituents are present as just described).

Unsubstituted or substituted polycyclic heterocyclyl is a heterocyclyl with two or more annealed rings, especially bi-, tri- or tetracyclic heterocyclyl, especially a bicyclic moiety as shown in the definition of heterocycyclyl in the formulae above or a moiety selected from the group represented by the following formulae:

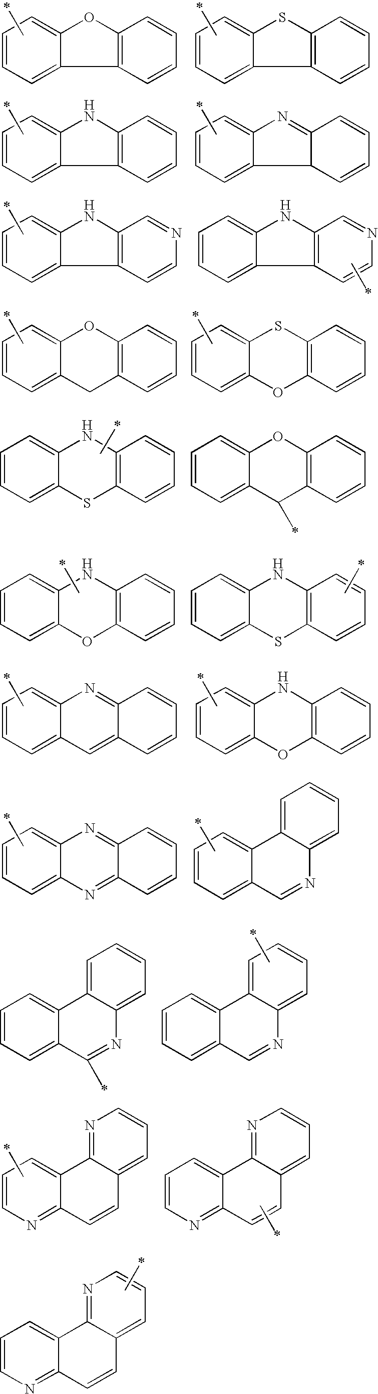

where each polycyclic heterocyclyl is unsubstituted or substituted by one or more, especial one to three, moieties independently selected from those mentioned as substituents for substituted heterocyclyl, especially C₁-C₇-alkyl, phenyl-C₁-C₇-alkyl or naphthyl-C₁-C₇-alkyl wherein the phenyl or naphthyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano; where the bond with asterisk marks the bond shown in formula I (and corresponding intermediates and starting materials) with which the respective moiety is bound to the rest of the molecule (so that any one H is present in such moiety may be replaced by that bond with the asterisk connecting the respective heterocyclyl moiety to the rest of the molecule, while one or more other H atoms may be replaced by a substituent if one or more substituents are present as just described).

Especially, unsubstituted or substituted polycyclic heterocyclyl is selected from the group consisting of indolyl, benzofuranyl, benzothienyl, quinolyl, isoquinolyl, carbazolyl, 9-thia-fluorenyl and 9-oxafluorenyl, each of which is unsubstituted or substituted by one or more, especial one to three, moieties independently selected from those mentioned as substituents for substituted heterocyclyl, especially C₁-C₇-alkyl, or phenyl-C₁-C₇-alkyl or naphthyl-C₁-C₇-alkyl wherein the phenyl or naphthyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano.

Unsubstituted or substituted cycloalkyl is preferably mono- or polycyclic, more preferably mono- or bicyclic, still more preferably monocyclic, C₃-C₁₆-, more preferably C₃-C₁₀-cycloalkyl, which may include one or more double (e.g. in cycloalkenyl) and/or triple bonds (e.g. in cycloalkinyl) with less double and/or triple bonds than required to form a fully unsaturated ring (e.g. aryl) system. Preferably, mono- or bicyclic cycloalkyl is saturated. The mono- or bicyclic cycloalkyl is unsubstituted or substituted by one or more, e.g. one to three substituents preferably independently selected from those mentioned above as substituents for aryl.

Acyl is preferably unsubstituted or substituted mono- or bicyclic aryl-carbonyl or -sulfonyl, unsubstituted or substituted mono- or bicyclic heterocyclylcarbonyl or -sulfonyl, unsubstituted or substituted mono- or bicyclic cycloalkylcarbonyl or -sulfonyl, formyl or (unsubstituted or substituted alkyl, unsubstituted or substituted mono- or bicyclic aryl-C₁-C₇-alkyl, unsubstituted or substituted mono- or bicyclic heterocyclyl-C₁-C₇-alkyl or unsubstituted or substituted mono- or bicyclic cycloalkyl-C₁-C₇-alkyl)-carbonyl or -sulfonyl, or (especially if bound to N, S or O) unsubstituted or substituted alkyloxycarbonyl, unsubstituted or substituted mono- or bicyclic aryl-oxycarbonyl, unsubstituted or substituted mono- or bicyclic heterocyclyloxycarbonyl, unsubstituted or substituted mono- or bicyclic cycloalkyloxycarbonyl, unsubstituted or substituted mono- or bicyclic aryl-C₁-C₇-oxycarbonyl, unsubstituted or substituted mono- or bicyclic heterocyclyl-C₁-C₇-oxycarbonyl, unsubstituted or substituted mono- or bicyclic cycloalkyl-C₁-C₇-oxycarbonyl or N-mono- or N,N-di-(unsubstituted or substituted mono- or bicyclic aryl, unsubstituted or substituted mono- or bicyclic heterocyclyl, unsubstituted or substituted mono- or bicyclic cycloalkyl, unsubstituted or substituted mono- or bicyclic aryl-C₁-C₇-alkyl, unsubstituted or substituted mono- or bicyclic heterocyclyl-C₁-C₇-alkyl, unsubstituted or substituted mono- or bicyclic cycloalkyl-C₁-C₇-alkyl or unsubstituted or substituted alkyl)-aminocarbonyl or -aminosulfonyl, with the proviso that—oxycarbonyl bound moieties are preferably bound to a nitrogen in the rest of the molecule. Preferred is C₁-C₇-alkanoyl, unsubstituted or mono-, di- or tri-(halo)-substituted benzoyl or naphthoyl, unsubstituted or phenyl-substituted pyrrolidinylcarbonyl, especially phenyl-pyrrolidinocarbonyl, C₁-C₇-alkylsulfonyl or (unsubstituted, halo-, C₁-C₇-alkyl- or halo-C₁-C₇-alkyl-substituted)-phenylsulfonyl, C₁-C₇-alkoxycarbonyl or phenyl-C₁-C₇-alkyloxycarbonyl. As acyl R2, indolyl-C₁-C₇-alkanoyl, e.g. indolylcarbonyl, quinolyl-C₁-C₇-alkanoyl, e.g. quinolinylcarbonyl, or phenyl-C₁-C₇-alkanoyl, e.g. phenylacetyl, wherein indolyl, quinolyl and phenyl are unsubstituted or substituted by a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, alkylene in each case may be straight-chained or branched and unsubstituted or (with lower preference) substituted e.g. by one or more moieties as defined for substituted alkyl, especially by halo, especially fluoro, hydroxy, C₁-C₇-alkoxy, phenyloxy, naphthyloxy, C₁-C₇-alkanoyloxy, benzoyloxy, naphthoyloxy, amino, mono- or di-(C₁-C₇-alkyl, C₁-C₇-alkanoyl, phenyl, naphthyl, phenyl-C₁-C₇-alkyl and/or naphthyl-C₁-C₇-alkyl)-amino, carboxy, C₁-C₇-alkoxycarbonyl or cyano, r and s, each independently of the other, are 0 or 1 and each of X and Y, if present and independently of the others, is —O—, —NV-, —S—, —O—CO—, —CO—O—, —NV-CO—; —CO-NV-; —NV-SO₂—, —SO₂—NV; —NV-CO-NV-, —NV-CO—O—, —O—CO-NV-, —NV-SO₂—NV- wherein V is hydrogen or unsubstituted or substituted alkyl as defined below, especially C₁-C₇-alkyl, or is phenyl, naphthyl, phenyl- or naphthyl-C₁-C₇-alkyl or halo-C₁-C₇-alkyl; and optionally one or more, e.g. up to two, further substituents selected from the other substituents mentioned for substituted aryl. Unsubstituted or substituted mono- or bicyclic aryl, unsubstituted or substituted mono- or bicyclic heterocyclyl, unsubstituted or substituted mono- or bicyclic cycloalkyl and unsubstituted or substituted alkyl are preferably as defined above whereever they are mentioned as part of acyl. “-Oxycarbonyl-” means —O—C(═O)—, “aminocarbonyl” means in the case of mono-substitution —NH—C(═O)—, in the case of double substitution also the second hydrogen is replaced by the corresponding moiety. For example, C₁-C₇-alkoxycarbonyl is C₁-C₇-alkyl-O—C(═O)—, N,N-di-(C₁-C₇-alkyl)aminocarbonyl is (C₁-C₇-alkyl)₂N—C(═O)—.

Generally, where substituents are present, they replace a hydrogen, e.g. in the case of R1 and/or R2.

The following preferred embodiments of the moieties and symbols in formula I can be employed independently of each other to replace more general definitions and thus to define especially preferred embodiments of the invention, where the remaining definitions of other moieties, respectively, can be kept broad as defined in embodiments of the inventions defined above of below.

### Preferred Definitions for R1

R1 is preferably selected from the group consisting of hydrogen, C₁-C₇-alkyl, especially C₁-C₄-alkyl, C₃-C₈-cycloalkyl, especially cyclopropyl or cyclobutyl, and C₃-C₈-cycloalkyl-C₁-C₄-alkyl, especially cyclopropylmethyl. Most preferably, R1 is C₃-C₈-cycloalkyl, especially cyclopropyl or cyclobutyl, most preferably cyclopropyl.

### Preferred Definitions for R2

In a first embodiment, R2 is preferably substituted alkyl, whereby preferred substituents are as defined herein. Preferred examples for alkyl are branched or straight chain C₁-C₇-alkyl. Preferred examples include methyl, ethyl, isopropyl, n-propyl, n-butyl, sec-butyl or tert-butyl, more preferably methyl, ethyl or isopropyl, most preferably methyl. The alkyl moiety is preferably mono-, di- or tri-substituted, more preferably mono-substituted. Suitable substituents for the alkyl moiety are as defined herein, preferably unsubstituted or substituted aryl or unsubstituted or substituted heterocylyl as defined herein.

The aryl moiety of the substituted alkyl is preferably phenyl or naphthyl, more preferably phenyl. When the aryl moiety is substituted, it is preferably mono- or di-substituted. Most preferably aryl is di-substituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, hydroxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, C₁-C₇-alkoxy-C₁-C₇-alkyl, C₁-C₇-alkoxy-C₁-C₇-alkoxy-C₁-C₇-alkyl, halo, such as fluoro, C₁-C₇-alkoxy, hydroxy-C₁-C₇-alkoxy and C₁-C₇-alkoxy-C₁-C₇-alkoxy, in particular C₁-C₇-alkoxy-C₁-C₇-alkoxy-C₁-C₇-alkyl, C₁-C₇-alkoxy, and C₁-C₇-alkoxy-C₁-C₇-alkoxy.

The heterocyclyl moiety of the substituted alkyl is preferably preferably mono- or bicyclic, more preferably bicyclic. Preferred are aromatic ring systems, or partially saturated ring systems, in particular whereby one of the rings is aromatic and the other is saturated or partially saturated, most preferred are aromatic. The heterocyclyl moiety has preferably 1, 2 or 3, more preferably 1 or 2, most preferably 1, heteroatoms selected from O, N or S, more preferably O or N. Particularly preferred examples include bicyclic 9- or 10-membered rings or monocyclic 5- or 6-membered rings, such as 10-membered rings preferably containing a nitrogen atom, in particular, quinolyl, isoquinolyl, 1,2,3,4-tetrahydro-1,4-benzoxazinyl, 2H-1,4-benzoxazin-3(4H)-onyl, 3,4-dihydro-1H-quinolin-2-onyl, or 4H-benzo[1,4]thiazin-3-onyl; bicyclic 9-membered ring systems preferably containing a N atom, in particular indolyl, 1H-indazolyl, benzothiophenyl, imidazo[1,2-a]pyridyl or 3H-benzooxazol-2-only; or 5- or 6-membered rings containing an N atom such as pyridyl, pyrrolyl and pyrimidinyl, more preferably heterocyclyl is indolyl or pyridyl most preferably indolyl, where each heterocyclyl is unsubstituted or substituted by one or more, e.g. up to three, preferably 1 or 2, substituents preferably independently selected from the group consisting of C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, hydroxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, C₁-C₇-alkoxy-C₁-C₇-alkyl, C₁-C₇-alkoxy-C₁-C₇-alkoxy-C₁-C₇-alkyl, halo, such as fluoro, C₁-C₇-alkoxy, hydroxy-C₁-C₇-alkoxy and C₁-C₇-alkoxy-C₁-C₇-alkoxy, in particular halo such as F, C₁-C₇-alkoxy-C₁-C₇-alkyl and hydroxy-C₁-C₇-alkoxy. Preferably heterocyclyl is substituted.

In a second embodiment R2 is preferably substituted or unsubstituted heterocyclyl. Heterocyclyl is preferably preferably mono- or bicyclic, more preferably bicyclic. Preferred are aromatic ring systems, or partially saturated ring systems, in particular whereby one of the rings is aromatic and the other is saturated or partially saturated, most preferred are partially saturated. The heterocyclyl moiety has preferably 1, 2 or 3, more preferably 1 or 2, most preferably 2, heteroatoms selected from O, N or S, more preferably O or N. The ring system contains preferably an oxo moiety. Particularly preferred examples include bicyclic 9- or 10-membered rings or monocyclic 5- or 6-membered rings, such as 10-membered rings preferably containing a nitrogen atom, in particular, quinolyl, isoquinolyl, 1,2,3,4-tetrahydro-1,4-benzoxazinyl, 2H-1,4-benzoxazin-3(4H)-onyl, 3,4-dihydro-1H-quinolin-2-onyl, or 4H-benzo[1,4]thiazin-3-onyl; bicyclic 9-membered ring systems preferably containing a N atom, in particular indolyl, 1H-indazolyl, benzothiophenyl, imidazo[1,2-a]pyridyl or 3H-benzooxazol-2-only; or 5- or 6-membered rings containing an N atom such as pyridyl, pyrrolyl and pyrimidinyl, more preferably heterocyclyl is 2H-1,4-benzoxazin-3(4H)-onyl, or 4H-benzo[1,4]thiazin-3-onyl; most preferably 2H-1,4-benzoxazin-3(4H)-onyl, where each heterocyclyl is unsubstituted or substituted by one or more, e.g. up to three, preferably 1 or 3, substituents preferably independently selected from the group consisting of C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, hydroxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, C₁-C₇-alkoxy-C₁-C₇-alkyl, C₁-C₇-alkoxy-C₁-C₇-alkoxy-C₁-C₇-alkyl, halo, such as fluoro, C₁-C₇-alkoxy, hydroxy-C₁-C₇-alkoxy and C₁-C₇-alkoxy-C₁-C₇-alkoxy, in particular halo such as F, and C₁-C₇-alkoxy-C₁-C₇-alkyl. Preferably heterocyclyl is substituted.

In one embodiment, R2 is preferably selected from phenyl, naphthyl, indolyl, phenyl-C₁-C₇-alkyl, naphthyl-C₁-C₇-alkyl, indolyl-C₁-C₇-alkyl and 2H-1,4-benzoxazin-3(4H)-onyl, where each phenyl, naphthyl, indolyl or 2H-1,4-benzoxazin-3(4H)-onyl is unsubstituted or preferably substituted by one or more, preferably up to three, especially up to two moieties independently selected from the group consisting of halo, especially fluoro, C₁-C₇-alkyloxy, C₁-C₇-alkyloxy-C₁-C₇-alkyl, C₁-C₇-alkyloxy-C₁-C₇-alkyloxy and C₁-C₇-alkyloxy-C₁-C₇-alkyloxy-C₁-C₇-alkyl.

In another embodiment, R2 is preferably phenyl, phenyl-C₁-C₇-alkyl, indolyl, indolyl-C₁-C₇-alkyl, 2H-1,4-benzoxazin-3(4H)-onyl, 2H-1,4-benzoxazin-3(4H)-onyl-C₁-C₇-alkyl, 4H-benzo[1,4]thiazin-3-onyl, 4H-benzo[1,4]thiazin-3-onyl-C₁-C₇-alkyl, pyridyl, and pyridyl-C₁-C₇-alkyl where each phenyl, indolyl, 2H-1,4-benzoxazin-3(4H)-onyl, 4H-benzo[1,4]thiazin-3-onyl, 4H-benzo[1,4]thiazin-3-only-C₁-C₇-alkyl, pyridyl, or pyridyl-C₁-C₇-alkyl is unsubstituted or substituted by one or more, especially up to three, moieties independently selected from C₁-C₇-alkyl, C₁-C₇-alkoxy-C₁-C₇-alkyl, C₁-C₇-alkoxy-C₁-C₇-alkoxy-C₁-C₇-alkyl, halo, such as fluoro, C₁-C₇-alkoxy, hydroxy-C₁-C₇-alkoxy and C₁-C₇-alkoxy-C₁-C₇-alkoxy.

### Preferred Definitions for W

In a first embodiment, W is unsubstituted or substituted polycyclic aryl. In this embodiment, polycyclic aryl is preferably naphthyl, fluorenyl or indenyl, most preferably naphthyl. When the aryl moiety is substituted, it is preferably mono- or di-substituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, carboxy-C₁-C₇-alkoxy, carboxy-C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, carboxy, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano, more preferably from C₁-C₇-alkyl, halo, such as F, carboxy-C₁-C₇-alkoxy, carboxy-C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, carboxy, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano. Most preferably, polycyclic aryl is unsubstituted.

In a preferred second embodiment, W is unsubstituted or substituted polycyclic heterocyclyl. In this embodiment, the heterocyclyl moiety is preferably bicyclic, tricyclic or tetracyclic, more preferably bicyclic or tricyclic, most preferably bicyclic. Preferred are aromatic ring systems, or partially saturated ring systems, in particular whereby one of the rings is aromatic and the other is saturated or partially saturated, most preferred are aromatic. The heterocyclyl moiety has preferably 1, 2 or 3, more preferably 1 or 2, most preferably 1, heteroatoms selected from O, N or S, more preferably S or N. Particularly preferred examples include bicyclic 9- or 10-membered rings or tricycyclic 12- to 14-membered rings, such as 9- or 10-membered rings preferably containing a nitrogen, oxygen or sulfur atom, in particular indolyl, 1H-indazolyl, benzothienyl, benzofuranyl, quinolyl, or isoquinolyl, or 13-membered rings such as carbazolyl, 9-oxa-fluorenyl and 9-thia-fluorenyl. When the heterocyclyl moiety is substituted, it is preferably mono- or di-substituted, more preferably mono-substituted. If the heterocyclyl moiety contains a nitrogen, the substitution is preferably on the nitrogen. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, carboxy-C₁-C₇-alkoxy, carboxy-C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, carboxy, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano, more preferably from C₁-C₇-alkyl, such as methyl, halo, such as F, carboxy-C₁-C₇-alkoxy, such as carboxy-(CH₂)₄—O—, carboxy-C₁-C₇-alkyl, such as carboxy-(CH₂)₄—, and phenyl-C₁-C₇-alkyl or naphthyl-C₁-C₇-alkyl, such as benzyl, wherein the phenyl or naphthyl is unsubstituted or substituted by up to three moieties independently selected from C₁-C₇-alkyl, halo, carboxy, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano, more preferably C₁-C₇-alkoxy, such as methoxy. Most preferably, polycyclic heteroaryl is unsubstituted, in particular if selected from tricyclic rings or benzofuranyl, benzothienyl, quinolyl, and isoquinolyl. Most preferably if the polycyclic heteroaryl is indolyl, it is substituted as described herein.

In one embodiment, W is preferably selected from the group consisting of naphthyl, indolyl, benzofuranyl, benzothienyl, quinolyl, isoquinolyl, carbazolyl, 9-oxa-fluorenyl and 9-thia-fluorenyl, each of which is unsubstituted or substituted by one or more, especially up to three, moieties independently selected from the group consisting of C₁-C₇-alkyl, C₁-C₇-alkoxy-C₁-C₇-alkyl, amino-C₁-C₇-alkyl, halo, especially fluoro, chloro or bromo, C₁-C₇-alkoxy and phenyl, naphthyl, phenyl-C₁-C₇-alkyl or naphthyl-C₁-C₇-alkyl wherein phenyl or naphthyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano.

In another embodiment, W is preferably indolyl, benzofuranyl, benzothienyl, quinolyl, isoquinolyl, carbazolyl, 9-thiafluorenyl or 9-oxafluorenyl, each of which is unsubstituted or substituted by one or more, especial one to three, moieties independently selected from C₁-C₇-alkyl, halo, such as F, carboxy-C₁-C₇-alkoxy, carboxy-C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, carboxy, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano.

### Preferred Definitions for R11

R11 is preferably hydroxy, halo, C₁-C₇-alkoxy, cyano or most especially hydrogen.

In all definitions above and below the person having skill in the art will, without undue experimentation or effort, be able to recognize which are especially relevant (e.g. those that if present provide compounds that are sufficiently stable for the manufacture of pharmaceuticals, e.g. having a half-life of more than 30 seconds, preferably of more than a week) and thus are preferably encompassed by the present claims and that only chemically feasible bonds and substitutions (e.g. in the case of double or triple bonds, hydrogen carrying amino or hydroxy groups and the like can be avoided in order to avoid tautomerism) are encompassed, as well as tautomeric forms where present, especially in equilibrium. For example, preferably, for reasons of stability or chemical feasibility, directly vicinal atoms in chains preferably are not selected from oxy plus oxy, thio plus oxy, oxy plus thio or thio plus thio, except where ring systems or the like are present that are sufficiently stable. Substitutents binding via an O (e.g. in C₁-C₇-alkoxy) or S that is part of them are preferably not bound to nitrogen e.g. in rings.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds”, “starting materials” and “intermediates” hereinbefore and hereinafter, especially to the compound(s) of the formula I or their precursors, is to be understood as referring also to one or more salts thereof or a mixture of a corresponding free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, starting materials, intermediates, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural (for example also different configuration isomers of the same compound, e.g. enantiomers in racemates or the like) or preferably the singular (“one”).

The compounds of the present invention can possess two or more asymmetric centers depending on the choice of the substituents. The preferred absolute configurations are as indicated herein specifically. However, any possible isolated or pure diastereoisomers, enantiomers or geometric enantiomers, and mixtures thereof, e.g., mixtures of enantiomers, such as racemates, are encompassed by the present invention.

As described above, the compounds of the present invention are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, Alzheimer's disease, dementia, anxiety states and cognitive disorders, and the like, especially where inhibition of (especially inappropriate) renin activity is required.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration and/or the like) and/or leads to or supports a renin dependent disease or disorder as mentioned above and below, e.g. by too high renin activity. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore “dependent” can be read as “dependent inter alia”, (especially in cases where a disease or disorder is really exclusively dependent only on renin) preferably as “dependent mainly”, more preferably as “dependent essentially only”. A disease dependent on (especially inappropriate) activity of renin may also be one that simply responds to modulation of renin activity, especially responding in a beneficial way (e.g. lowering the blood pressure) in case of renin inhibition.

Where a disease or disorder dependent on (=that “depends on”, “depending”) (especially inappropriate) activity of a renin is mentioned (such in the definition of “use” in the following paragraph and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural renin and/or one or more altered or mutated forms thereof.

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more diseases or disorders mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate, and each of the more specific definitions, independent of any others, may be introduced independently of or together with one or more other more specific definitions for other more general expressions or symbols.

In a first preferred embodiment, the invention especially relates to a compound of the formula I wherein

R1 is hydrogen, alkyl, cycloalkyl substituted alkyl or cycloalkyl;
R2 is substituted alkyl where the substituents are selected from unsubstituted or substituted aryl and unsubstituted or substituted heterocyclyl, or is unsubstituted or substituted heterocyclyl;
W is unsubstituted or substituted polycyclic heterocyclyl or unsubstituted or substituted polycyclic aryl; and
R11 is hydrogen;
or a pharmaceutically acceptable salt thereof;
or the use of such compound or salt according to the invention.

More preferably, the invention relates to a compound of the formula I, wherein

R1 is hydrogen, C₁-C₇-alkyl, C₃-C₈-cycloalkyl or C₃-C₈-cycloalkyl-C₁-C₇-alkyl;
R2 is phenyl, phenyl-C₁-C₇-alkyl, indolyl, indolyl-C₁-C₇-alkyl, 2H-1,4-benzoxazin-3(4H)-onyl, 2H-1,4-benzoxazin-3(4H)-onyl-C₁-C₇-alkyl, where each phenyl, indolyl or 2H-1,4-benzoxazin-3(4H)-onyl is unsubstituted or substituted by one or more, especially up to three, moieties independently selected from C₁-C₇-alkyl, C₁-C₇-alkoxy-C₁-C₇-alkyl, C₁-C₇-alkoxy-C₁-C₇-alkoxy-C₁-C₇-alkyl, halo, such as fluoro, C₁-C₇-alkoxy and C₁-C₇-alkoxy-C₁-C₇-alkoxy;
W is naphthyl, indolyl, benzofuranyl, benzothienyl, quinolyl, isoquinolyl, carbazolyl, 9-thia-fluorenyl or 9-oxafluorenyl, each of which is unsubstituted or substituted by one or more, especial one to three, moieties independently selected from C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano;
and R11 is hydrogen;
or a (preferably pharmaceutically acceptable) salt thereof; or the use of such compound or salt according to the invention.

Very preferred among the preceding and following compounds of the formula I are those that have the configuration given in the following formula IA,

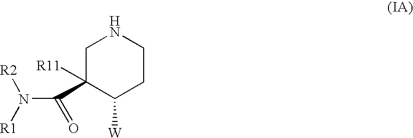

wherein R1, R2, R11 and W are as defined hereinabove or hereinbelow, or (preferably pharmaceutically acceptable) salts thereof.

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as the use thereof according to the invention.

### Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by reacting a carbonic acid of the formula II,

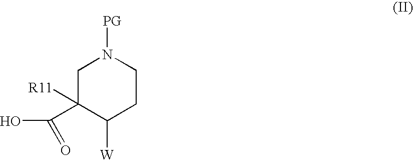

or a reactive derivative thereof, wherein PG is a protecting group and W and R11 are as defined for a compound of the formula I, with an amino compound of the formula III,

R1—NH—R2 (III)

wherein R1 and R2 are as defined for a compound of the formula I,
and, if desired, subsequent to this condensation reaction, converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials of the formula II and/or Ill, in addition to specific protecting groups mentioned, further protecting groups may be present, and any protecting groups are removed at an appropriate stage (especially before or after a reaction mentioned under “if desired”) in order to obtain a corresponding compound of the formula I, or a salt thereof.

### Preferred Reaction Conditions

The preferred reaction conditions for the reactions mentioned above, as well as for the transformations and conversions, are as follows (or analogous to methods used in the Examples or as described there):

The condensation of a carbonic acid of the formula II, or a reactive derivative thereof, preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also and preferably be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine, diisopropylethylamine (DIEA) or N-methylmorpholine and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCI); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP), 1-(3-dimethylaminopropyl)-3-ethylcarbodiimide hydrochloride/hydroxybenzotriazole or/1-hydroxy-7-azabenzotriazole (EDC/HOBT or EDC/HOAt), HOAt alone, 4-(4,6-dimethoxy-1,3,5-triazin-2-yl)-4-methylmorpholinium chloride (DMT-MM) or with 1-chloro-2-methyl-propenyl)-dimethylamine (=1-chloro-N,N,2-trimethyl-1-propenylamine). For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction may preferably carried out under an inert gas, e.g. nitrogen or argon.

In order to obtain a compound of the formula I if no further conversion in the protected state is desired, the subsequent removal of a protecting group, e.g. PG, such as tert-butoxycarbonyl, benzyl, 9H-fluoren-9-ylmethoxycarbonyl or 2-(trimethylsilyl)-ethoxycarbonyl, takes place under standard conditions, see also the literature mentioned below under General Process Conditions. For example, tert-butoxycarbonyl is removed in the presence of an acid, e.g. a hydrohalic acid, such as HCl, in an appropriate solvent, e.g. an ether, such as dioxane, or an alcohol, e.g. isopropanol, at customary temperatures, e.g. at room temperature, the removal of benzyl can be achieved e.g. by reaction with ethylchloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., or by removal by means of trimethylsilyl trifluoroacetate in the presence of a tertiary nitrogen base, such as 2,6-lutidine, in an appropriate solvent, such as a halogenated hydrocarbon, e.g. methylene chloride; the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetonitrile, preferably at elevated temperatures, e.g. under reflux conditions, and the removal of a 9H-fluoren-9-yl-methoxycarbonyl protecting group can be achieved in the presence of a secondary amine, especially piperidine, in an appropriate solvent, e.g. a halogenated hydrocarbons, such as methylene chloride, at preferred temperatures between 0 and 50° C., e.g. at about room temperature.

### Optional Reactions and Conversions

A compound of the formula I, or a protected form thereof directly obtained according to any one of the preceding procedures (meaning that, if conversion is desired, a removal of protecting groups is not required in the above-mentioned condensation reaction or after introducing protecting groups anew) which are included subsequently as starting materials for conversions as well even if not mentioned specifically, can be converted into different compounds of the formula I according to known procedures, where required or desired after removal of protecting groups.

Where R1 is hydrogen in a compound of the formula I, this can be converted into the corresponding compound wherein R1 has a meaning other than hydrogen given for compounds of the formula I by reaction with a compound of the formula IV,

R1*-Q (IV)

wherein R1* is defined as R1 in a compound of the formula I other than hydrogen and Q is a leaving group (e.g. preferably selected from halo, e.g. chloro, from unsubstituted or substituted aryl-sulfonyloxy, such as toluolsulfonyloxy, from unsubstituted or substituted alkylsulfonyloxy, such as methylsulfonyloxy or trifluoromethylsulfonyloxy, with the reaction allowed to take place e.g. in the presence of a base, such as an alkali metal salt of a weaker acid, e.g. an alkali metal carbonate and/or an alkali metal hydrogencarbonate, such as sodium or potassium carbonate and/or sodium or potassium hydrogencarbonate (NaHCO₃ or KHCO₃) in an appropriate solvent, e.g. dioxane and/or H₂O, at preferred temperatures between −20 and 50° C., e.g. at −5 to 30° C.), or wherein Q is —CHO (so that the compound of the formula IV is an aldehyde) and then R1* is the complementary moiety for a moiety R1 that includes a methylene group (resulting in a group R1 of the formula R1*—CH₂—) e.g. under reductive amination conditions as follows: The reaction preferably takes place under customary conditions for reductive amination, e.g. in the presence of an appropriate hydrogenation agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature.

Hydroxy substituents, e.g. as substitutents of aryl in alkyl substituted by aryl R1, R2 or in other aryl substituents, can be transformed into unsubstituted or substituted alkoxy, e.g. by alkylation reaction with the corresponding unsubstituted or substituted alkylhalogenide, e.g. iodide, in the presence of a base, e.g. potassium carbonate, in an appropriate solvent, e.g. N,N-dimethylformamide, e.g. at preferred temperatures between 0 and 50° C.

Carboxy substitutents can be converted into esterified carboxy by reaction with correspondding alcohols, e.g. C₁-C₇-alkanols, or into amidated carboxy by reaction with corresponding amines, e.g. under condensation conditions analogous to those described above under the condensation reaction between a compound of the formula II and a compound of the formula III.

Esterified carboxy substituents can be converted into free carboxy by hydrolysis, e.g. in the presence of a base, such as potassium hydroxide, in an appropriate solvent, e.g. tetrahydrofurane, preferably at elevated temperatures, e.g. from 50° C. to the reflux temperature of the reaction mixture.

In some cases, the conversions preferably take place with compounds of the formula I in protected form; the subsequent removal of protecting group can be achieved as described above for the condensation reaction between a compound of the formula II and a compound of the formula III and below under “General Process Conditions”, yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers or enantiomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-)crystallization, and the like. Some possible methods that can also be used with other compounds analogously can be found in the Examples.

### Starting Materials

In the subsequent description of starting materials (which term also includes intermediates) and their synthesis, R1, R2, R2*, R11, W and PG have the meanings given above (especially for compounds of the formulae I, II, III or IV) or in the Examples for the respective starting materials or intermediates, if not indicated otherwise directly or by the context. Protecting groups, if not specifically mentioned, can be introduced and removed at appropriate steps or stages in order to prevent functional groups, the reaction of which is not desired in the corresponding reaction step or steps, from participating in a reaction, employing protecting groups, methods for their introduction and their removal are as described above or below, e.g. in the references mentioned under “General Process Conditions”. The person skilled in the art will readily be able to decide whether and which protecting groups are useful or required and at which stage it is appropriate to introduce, exchange and/or remove protecting groups.

A compound of the formula II wherein R11 is hydrogen can, for example, be prepared by reducing a tetrahydropyridine compound of the formula V,

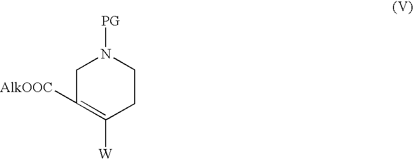

wherein Alk is the moiety of an alcohol, e.g. methyl or ethyl, to the corresponding compound of the formula II wherein R11 is hydrogen. The reduction can take place under customary conditions, for example (i) with hydrogen in the presence of a noble metal catalyst, e.g. in dispersion such as Pd on charcoal or with a homogenous catalyst such as Pd(OAc)₂, in an appropriate solvent, for example an alcohol, such as ethanol, or N-methylpyrrolidone, or mixtures of two or more thereof, at preferred temperatures in the range from 0 to 50° C., e.g. at room temperature; (ii) in the presence of a complex hydride, especially sodium borohydride, and e.g. NiCl₂ in an appropriate solvent, such as an alcohol, e.g. at temperatures from −30 to 30° C.; or (iii) in the presence of a reducing metal, such as Mg, in an appropriate solvent, e.g. an alcohol, such as methanol, at preferred temperatures from −20 to 40° C., resulting in a compound of the formula VI,

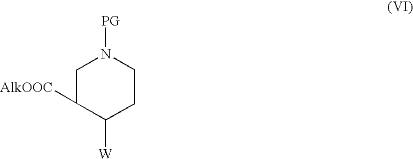

which can then, if desired under epimerization, preferably be hydrolyzed to the corresponding compound of the formula II wherein the carboxy group and W are present in the configuration of the R1R2N—C(═O)— and the W in formula IA given above, be converted to the corresponding compound of the formula II, e.g. (i) in the presence of an alcoholate of the formula MetOAlk, where Met is preferably an alkali metal, e.g. Na, and Alk is as defined under formula V, in the presence of an appropriate solvent, e.g. the corresponding alcohol AlcOH, e.g. methanol or ethanol, to achieve epimerization, followed by hydrolysis with water, e.g. at elevated temperatures from 30 to 80° C. or under reflux, or (ii) by addition of a metal hydroxide, e.g. potassium hydroxide, in the presence of water at elevated temperatures, e.g. from 50° C. to the reflux temperature of the mixture.

A tetrahydropyridine compound of the formula V can, for example, be prepared by reacting a compound of the formula VII,

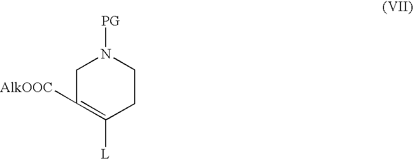

wherein L is a leaving group, e.g. as described for a compound of the formula IV, and the other moieties have the meanings described for a compound of the formula V, with a compound of the formula VIII,

W-X (VIII)

wherein W is as described for a compound of the formula I and X is —B(OH)₂ or —B(OY)₂ wherein the two Y together form a methylene, an ethylene or a corresponding bridge that is substituted by up to four methyl moieties, e.g. a pinakol borate group, or a leaving group as defined for a compound of the formula III, under customary reaction conditions, e.g. if the reaction takes place with a compound of the formula VII wherein L is a leaving group and with a compound of the formula VIII wherein X is —B(OH)₂ or —B(OY)₂, L is preferably halo, such as bromo or iodo, or trifluoromethylsulfonyloxy, and the reaction preferably takes place in an appropriate solvent, such as dioxane in the presence or absence of water, a basic buffering substance, e.g. potassium phosphate or potassium carbonate, and catalyst, e.g. Pd(PPh₃)₄, at preferably elevated temperatures, e.g. between 50° C. and the reflux temperature of the mixture. Where the reaction under takes place with a compound of the formula VII wherein L is hydroxy and with a compound of the formula VIII wherein X is a leaving group, the leaving group is preferably halo, e.g. bromo or iodo, and the coupling reaction preferably takes place in the presence of a base, such as potassium carbonate, in an appropriate solvent, e.g. N,N-dimethylformamide, at preferably elevated temperatures, e.g. from 30 to 80° C. Removal of protecting groups can take place as described above for removal of protecting groups after reaction of compounds of the formulae II and III and below in the general process conditions.

Hydroxy R11 can be introduced into starting materials at various stages, e.g. in (preferably appropriately protected) compounds of the formula VI, for example by treatment with a strong base to remove the hydrogen to be substituted by R11, such as lithium hexamethyldisialazide (LHMDS) or preferably lithium diisopropylamide in tetrahydrofuran at low temperatures, e.g. from −100 to −50° C., such as at −78° C., followed by oxidation e.g. by addition of an oxaziridine derivative according to Davis (e.g. 2-tert-butoxycarbonyl-3-trichloromethyl-oxaziridine or 2-(phenylsulfonyl or tolylsulfonyl)-3-phenyl-oxaziridine or (e.g. for stereoselective synthesis) (+)- or (−)-(camphorsulfonyl)oxaziridine) to give the corresponding hydroxy compound, see e.g. Julia Marc.; Bulletin de la Societe Chimique de France, 1996, 15-24.

Hydroxy R11 may then further be esterified or etherified according to standard procedures to give the corresponding compounds wherein R11 is halo, C₁-C₇-alkoxy, halo-C₁-C₇-alkoxy or cyano.

Alternatively, the compound of the formula VI can be treated with a strong base as just mentioned and then reacted with a C₁-C₇-alkylhalogenide, a cycloalkylhalogenide, a halo-C₁-C₇-alkyltosylate or a halo-cycloalkyltosylate to introduce the corresponding moieties C₁-C₇-alkyl, halo-C₁-C₇-alkyl, cycloalkyl or halo-substituted cycloalkyl, see e.g. Taylor, G. Marc.; Tetrahedron Letters, 1996, 1297-1300 and Coppola, Gary M.; Bioorganic and Medicinal Chemistry Letters, 2002, 2439-2442.

A compound of the formula III may, for example, be prepared by reacting an amino compound of the formula IX,

R1—NH₂ (IX)

wherein R1 is as defined for a compound of the formula I with an aldehyde of the formula X,

R2*—CHO (X)

wherein R2* is the complementary moiety for a moiety R2 that includes a methylene group (resulting in a group R2 of the formula R2*—CH₂—) e.g. under reaction conditions as follows: The corresponding reaction (reductive amination) can take under customary conditions, e.g. in the presence of an appropriate hydrogenation agent, such as hydrogen in the presence of a catalyst or a complex hydride, e.g. sodium triacetoxyborohydride or sodium cyanoborohydride, in an appropriate solvent, such as a halogenated hydrocarbon, e.g. methylene chloride or 1,2,-dichloroethane, and/or an alcohol, such as methanol, and optionally a carbonic acid, e.g. acetic acid, at preferred temperatures between −10° C. and 50° C., e.g. from 0° C. to room temperature.

In a compound of the formula X wherein R2* is a heterocyclyl comprising an NH in the ring, such as indolyl, the H can be replaced with unsubstituted or substituted alkyl by reaction with a corresponding (unsubstituted or substituted alkyl)-halogenide or -tosylate (toluolsulfonyloxy-group comprising), e.g. in the presence of a base, such as sodium or potassium hydride, a corresponding halogenide salt, e.g. potassium iodide, and an appropriate solvent, e.g. N,N-dimethylformamide or the like, at temperatures e.g. in the range from −10 to 50° C., e.g. from 0 to 25° C., giving the corresponding compound of the formula X with an N-bound unsubstituted or substituted alkyl.

Comparably, where in a compound of the formula VIII W is a polycyclic heterocyclyl comprising an NH as ring element, the H can be replaced by unsubstituted or substituted alkyl, such as (C₁-C₇-alkyloxy-mono- or disubstituted phenyl)-C₁-C₇-alkyl, by reaction of the corresponding unsubstituted or substituted alkyl-halogenide, e.g. bromide, under conditions comparable to those just mentioned for a compound of the formula X in the preceding paragraph.

A compound of the formula VIII wherein W is as described for a compound of the formula I and X is —B(OY)₂ can be prepared from a corresponding compound of the formula VIII*,

W-X* (VIII*)

wherein X* is halo, e.g. bromo, by reaction with a compound of the formula XI,

(YO)₂B-B-(OY)₂ (XI)

wherein Y is as described above in the presence of a base, e.g. an alkali metal acetate, such as potassium acetate, and an appropriate catalyst, e.g. PdCl₂(dppf), in an appropriate solvent, such as dimethylsulfoxide, and under customary temperature conditions, e.g. temperatures from 0 to 50° C., e.g. at room temperature.

A compound of the formula X can be obtained by reducing a corresponding hydroxymethylene precursor of the formula XII,

R2*—CH₂—OH (XII)

under appropriate conditions, e.g. in the presence of manganese dioxide and an appropriate solvent, e.g. an ester, such as ethyl acetate, at appropriate temperatures, e.g. in the range from 20 to 80° C., e.g. at about 60° C.

A hydroxymethylene compound of the formula XII can, for example, be obtained from a carbonic acid ester of the formula XIII,

R2*—COOAlk (XIII)

wherein Alk is the moiety of an alcohol, e.g. of methyl or ethyl, by reduction under appropriate conditions, e.g. in the presence of an appropriate complex hydride, such as lithium aluminium hydride, in a customary solvent, such as a cyclic ether, e.g. tetrahydrofurane, at temperatures e.g. from −30 to 50° C., e.g. at about 0° C.

In a compound of the formula X or XIII wherein R2* is substituted aryl carrying a hydroxymethylene group (and possibly other substituents), the hydroxymethylene group can be reacted with an unsubstituted or substituted alkyl-tosylate, e.g. a C₁-C₇-alkoxy-C₁-C₇-tosylate, e.g. in the presence of a base, such as sodium or potassium hydride, a corresponding halogenide salt, e.g. potassium iodide, and an appropriate solvent, e.g. N,N-dimethylformamide or the like, at temperatures e.g. in the range from −10 to 50° C. C, e.g. from 0 to 25° C., giving the corresponding compound of the formula X or XII carrying an (further unsubstituted or substituted) aryl with a corresponding unsubstituted or substituted alkyloxy-methyl substituent, e.g. C₁-C₇-alkoxy-C₁-C₇-alkoxy-methyl.

Other starting materials, such as also starting materials of the formula II, IV, V, VI, VII, VIII, IX, X, XI, XII or XIII, are known in the art, can be prepared according to methods that are known in the art and/or are commercially available, or they can be prepared in analogy to methods described in the Examples given below.

### General Process Conditions

The following applies in general (where possible) to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the processes in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the processes of the present invention those starting materials are preferably used which result in compounds of formula I described as being preferred. Special preference is given to reaction conditions that are identical or analogous to those mentioned in the Examples. The invention relates also to novel starting compounds and intermediates described herein, especially those leading to novel compounds of the formula I or compounds of the formula I mentioned as preferred herein.

### Pharmaceutical Use, Pharmaceutical Preparations and Methods

As described above, the compounds of the formula I are inhibitors of renin activity and, thus, may be of use for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like. Hypertension, at least as one component of the disease to be treated, is especially preferred, meaning that hypertension alone or in combination with one or more (especially of the mentioned) other diseases may be treated (prophylactically and/or therapeutically).

The present invention further provides pharmaceutical compositions comprising a therapeutically effective amount of a pharmacologically active compound of the formula I, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders and the like. Especially preferred is a disease which comprises hypertension, more especially hypertension itself, where treatment with a pharmaceutical composition or the use of a compound of the formula I for its synthesis is useful prophylactically and/or (preferably) therapeutically.

Thus, the pharmacologically active compounds of the formula I may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin. Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) anti-obesity agents such as orlistat; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibittors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the formula I may be administered either simultaneously, before or after the other active ingredient, either separately by the same or different route of administration or together in the same pharmaceutical formulation. The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical products or compositions comprising a therapeutically effective amount of a compound of the formula I alone or in combination with a therapeutically effective amount of another therapeutic agent, preferably selected from anti-diabetics, hypolipidemic agents, anti-obesity agents and anti-hypertensive agents, most preferably from antidiabetics, anti-hypertensive agents and hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material.

The present invention further provides a method for the prevention and/or treatment of conditions mediated by (especially inappropriate) renin activity, which comprises administering a therapeutically effective amount of a compound of the formula I to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a pharmaceutical product comprising a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity, especially one or more of the specific diseases mentioned above.

Finally, the present invention provides a method or use which comprises administering a compound of formula I in combination with a therapeutically effective amount of an anti-diabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodiumion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-ProPhe-His-Leu-Val-Ile_His_Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 20 μM.

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)-IleHis-Pro-Phe-His-Leu-Val-Ile_His_Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 20 μM. In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably can show IC₅₀ values in the range from 1 nM to 20 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-HisLeu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably show IC₅₀ values in the range from 1 nM to 20 μM.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

Compounds of the formula I can be tested in vivo in primates as described in the literature (see for example by Schnell C R et al. Measurement of blood pressure and heart rate by telemetry in conscious, unrestrained marmosets. Am J Physiol 264 (Heart Circ Physiol 33). 1993: 1509-1516; or Schnell C R et al. Measurement of blood pressure, heart rate, body temperature, ECG and activity by telemetry in conscious, unrestrained marmosets. Proceedings of the fifth FELASA symposium: Welfare and Science. Eds BRIGHTON. 1993.

The following Examples, while in addition representing preferred embodiments of the invention, serve to illustrate the invention without limiting its scope.

5-(4,4,5,5-tetramethyl-1,3,2-dioxaborolan-2-yl)-1-benzofuran is from MAYBRIDGE, quinoline-6-boronic acid from ASYMCHEM, 3-quinoline boronic acid from ACROS. The other all boronic acids are from ALDRICH.

### Abbreviations

Ac acetyl
aq. aqueous
Boc tert-butoxycarbonyl
Brine saturated sodium chloride solution
Celite trademark of Celite Corp. for filtering aid based on kieselguhr
conc. concentrated
DCM dichloromethane
DIEA N,N-diisopropylethylamine
DMF N,N-dimethylformamide
DMSO dimethylsulfoxide
DMT-MM 4-(4,6-dimethoxy-1,3,5-triazin-2-yl)-4-methylmorpholinium chloride
EDC 1-(3-Dimethylaminopropyl)-3-ethylcarbodiimide hydrochloride
ES-MS electrospray mass spectrometry
Et ethyl
ether diethyl ether
EtOAc ethyl acetate
h hour(s)
HOAt 1-hydroxy-7-azabenzotriazole
HPLC high-pressure liquid chromatography
IPr isopropyl
LAH lithium aluminium hydride
Me methyl
min minute(s)
mL milliliter(s)
MOMCl methoxymethyl chloride
MS Mass Spectrometry
MsCl Methylsulfonylchlorid
n-Hex n-hexyl
NaOMe sodium methoxylate
NMR nuclear magnetic resonance
Ph phenyl
RT room temperature
TBTU O-(benzotriazol-1-yl)-N,N,N′,N′-tetramethylammonium tetrafluoroborate
TFA trifluoroacetic acid
THF tetrahydrofurane
TMS trimethylsilyl
TMSOTf trifluoromethanesulfonic acid trimethylsilyl ester
WSCD=EDC
t_{Ret} HPLC retention time in min determined by HPLC condition

### Synthesis

Flash chromatography is performed by using silica gel (Merck; 40-63 μm). For thin layer chromatography, pre-coated silica gel (Merck 60 F254; Merck KgaA, Darmstadt, Germany)) plates are used. ¹NMR measurements are performed on a Bruker DXR 400 spectrometer using tetramethylsilane as internal standard. Chemical shifts (δ) are expressed in ppm downfield from tetramethylsilane. Electrospray mass spectra are obtained with a Fisons Instruments VG Platform II. Commercially available solvents and chemicals are used for syntheses.

### HPLC Condition

### Column: Nucleosil 100-3 C18 HD, 125×4.0 mm.

Flow rate: 1.0 ml/min
Mobile phase: A) TFA/water (0.1/100, v/v), B) TFA/acetonitrile (0.1/100, v/v)
Gradient: linear gradient from 20% B to 100% B in 7 min

### Detection: UV at 254 nm

The HPLC conditions can be identified by the subscript prefixes of the T_{Ret} values given in the examples.

In the following examples, only one (the preferred) configuration is shown, while in fact enantiomer mixtures are present. The single enantiomers can be obtained e.g. by classical crystallization of diastereomeric salt with enantiomer pure salt forming chiral ions or chiral chromatography.

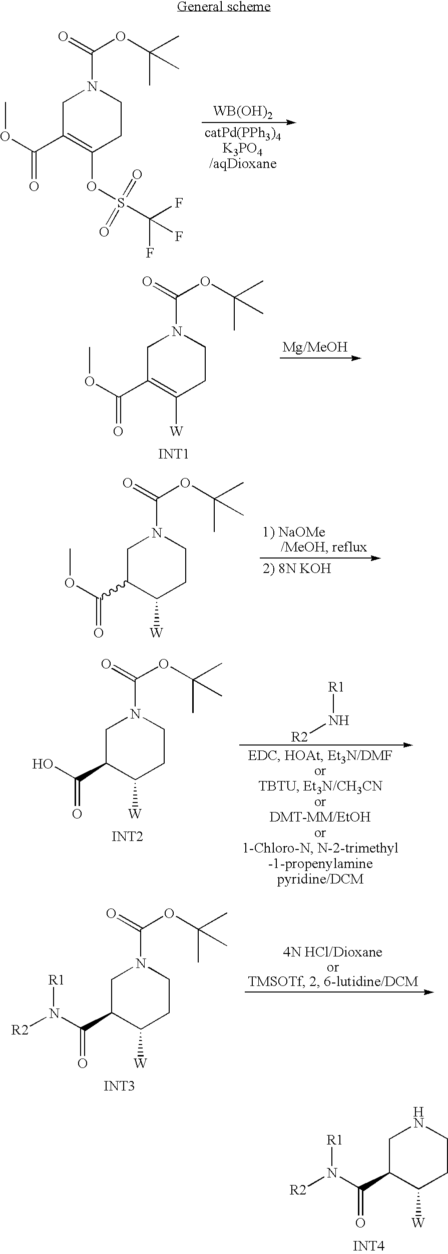

Intermediates INT2, INT3, INT4 are obtained as a racemic mixture, or optical resolution of INT2 using an appropriate chiral amine (such as cinchonidine, cinchonine, quinine or quinidine) affords corresponding enantiomeric pure INT2. And INT3 or the final product INT4 can be separated into the pure enantiomers by common techniques like chiral chromatography.

### Example 1

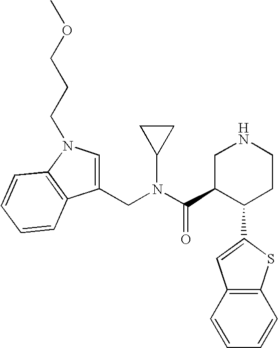

A mixture of Intermediate 1.1 (127 mg, 0.21 mmol) and 2,6-lutidine (86 μL, 0.74 mmol) in DCM (3 mL) is treated with TMSOTf (96 μL, 0.53 mmol) at 0° C. After stirring for 30 min, aqueous saturated NaHCO₃ and MeOH are added. Then EtOAc is added and the organic layer is washed with brine, dried over MgSO₄ and evaporated in vacuo. Silica gel flash chromatography of the residue affords Example 1 as white solid; ES-MS: M+H=502; HPLC: t_{Ret}=3.50 min.

### Intermediate 1.1:

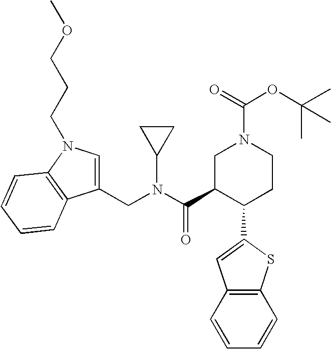

A mixture of Intermediate 1.4 (153 mg, 0.42 mmol), Intermediate 1.2 (131 mg, 0.51 mmol), EDC (121 mg, 0.63 mmol) and HOAt (86 mg, 0.63 mol) in DMF (1.5 mL) is stirred for 1.5 h at 60° C. H₂O and Et₂O are added and the organic layer is washed with brine, dried over MgSO₄ and evaporated in vacuo. Silica gel flash chromatography of the residue (hexane/ethyl acetate) affords Intermediate 1.1 as white amorphous material; ES-MS: M+H=602; HPLC: t_{Ret}=5.55 min.

### Intermediate 1.2:

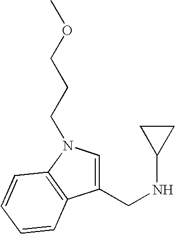

A mixture of Intermediate 1.3 (780 mg, 3.6 mmol), cyclopropylamine (410 mg, 7.2 mmol), AcOH (0.5 mL) and NaBH(OAc)₃ (1.1 g, 5.4 mmol) in DCM (3 mL) and MeOH (1 mL) is stirred under N₂ at 0° C. After stirring at RT for 1 h, the reaction mixture is quenched with saturated aqueous NaHCO₃ and extracted with DCM. The combined organic phases are washed with H₂O, brine and dried (Na₂SO₄). Concentration under reduced pressure and silica gel flash chromatography give Intermediate 1.2 as yellow oil; ES-MS: M+H=202; HPLC: t_{Ret}=2.67 min

### Intermediate 1.3:

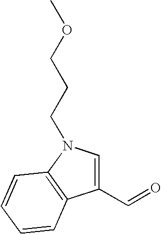

To a mixture of indole-3-carboxaldehyde (1.0 g, 6.9 mmol), toluene-4-sulfonic acid 3-methoxy-propyl ester (2.1 g, 9.0 mmol) and KI (1.1 g, 7.0 mmol) in DMF (15 mL), NaH (320 mg, 7.5 mmol) is added under N₂ at 0° C. After stirring at 50° C. for 4 h, the H₂O is added to the reaction mixture which is then extracted with EtOAc. The combined organic phases are washed with H₂O, brine and dried (Na₂SO₄). Concentration under reduced pressure and silica gel flash chromatography give Intermediate 1.3 as colorless oil; ES-MS: M+H=218, HPLC: t_{Ret}=3.18 min.

### Intermediate 1.4:

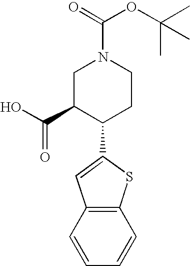

To a solution of Intermediate 1.5 (672 mg, 1.88 mmol) in dioxane (3.8 mL), 8N KOH (3.8 mL) is added. After refluxing for 2 h, the reaction mixture is cooled down to RT, adjusted to weakly acidic pH by slowly adding critic acid and extracted with EtOAc. The combined organic phases are washed with brine and dried (MgSO₄). Concentration under reduced pressure and purified by silica gel flash chromatography give Intermediate 1.4 as white amorphous material; ES-MS: M-^{t}Bu=306; HPLC: t_{Ret}=4.22 min.

### Intermediate 1.5:

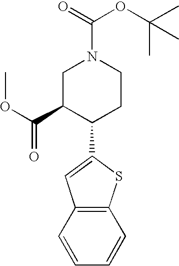

To a solution of Intermediate 1.6 (834 mg, 2.23 mmol) in MeOH (22.3 mL), Mg (651 mg, 26.8 mmol) is added at 0° C. under N₂. After stirring at RT for overnight, the reaction mixture is filtered through Celite pad and diluted with EtOAc. The reaction mixture is washed with saturated aqueous NH₄Cl and brine, and dried (MgSO₄). Concentration under reduced pressure follows. The residue and NaOMe (2.0 mL, 25 wt % MeOH solution, 9.28 mmol) are dissolved in MeOH. After adding H₂O, the reaction mixture is extracted with EtOAc. The combined organic phases are washed with brine and dried (MgSO₄). Concentration under reduced pressure gives Intermediate 1.5 as white amorphous material; ES-MS: M-^{t}Bu=320; HPLC: t_{Ret}=4.91 min.

### Intermediate 1.6:

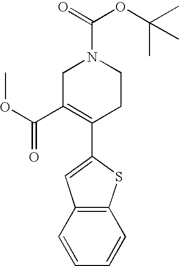

A mixture of 4-trifluoromethanesulfonyloxy-5,6-dihydro-2H-pyridine-1,3-dicarboxylic acid 1-tert-butyl ester 3-methyl ester (1.84 g, 4.73 mmol) (see e.g. WO 2004/002957 or US 2003/216441), 1-benzothiophene boronic acid (1.01 g, 5.67 mmol), K₃PO₄ (1.20 g, 5.68 mmol) and Pd(PPh₃)₄ (546 mg, 0.47 mmol) in dioxane (19 mL) and H₂O (3.8 mL) is stirred at 60° C. for 2 h. After adding H₂O, the reaction mixture is extracted with EtOAc. The combined organic phases are washed with H₂O, brine and dried (MgSO₄). Concentration under reduced pressure and silica gel flash chromatography give Intermediate 1.6 as white amorphous material; ES-MS: M-^{t}Bu=318; HPLC: t_{Ret}=4.89 min.

### Example 2

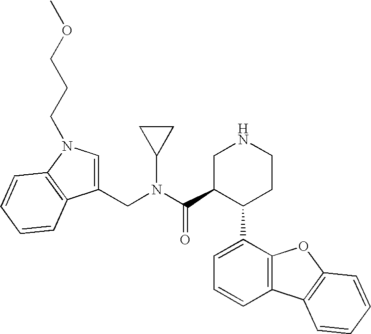

Example 2 is synthesized by deprotection of Intermediate 2.1 (210 mg, 0.33 mmol) analogously to the preparation of Example 1. White solid; ES-MS: M+H=536; HPLC: t_{Ret}=3.67 min.

### Intermediate 2.1:

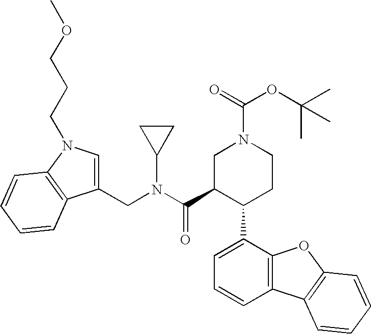

Intermediate 2.1 is synthesized by condensation of Intermediate 2.2 (200 mg, 0.50 mmol) and Intermediate 1.2 (170 mg, 0.66 mmol) analogously to the preparation of Intermediate 1.1. White solid; ES-MS: M+H=636; HPLC: t_{Ret}=5.67 min.

### Intermediate 2.2:

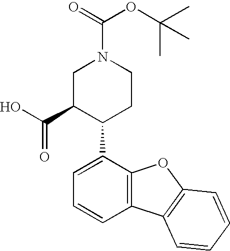

Intermediate 2.2 is synthesized by 1,4-reduction, epimerization and hydrolysis of Intermediate 2.3 (2.15 g, 5.13 mmol) analogously to the preparation of Intermediate 1.5 and 1.4. White solid; ES-MS: M-^{t}Bu=340; HPLC: t_{Ret}=4.49 min.

### Intermediate 2.3:

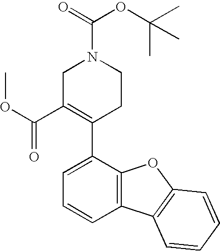

Intermediate 2.3 is synthesized by condensation of 4-trifluoromethanesulfonyloxy-5,6-dihydro-2H-pyridine-1,3-dicarboxylic acid 1-tert-butyl ester 3-methyl ester (2.0 g, 5.13 mmol) and dibenzofuran-4-boronic acid (1.41 g, 6.68 mmol) analogously to the preparation of Intermediate 1.6. Colorless solid; ES-MS: M+H=352; HPLC: t_{Ret}=5.07 min.

### Example 3

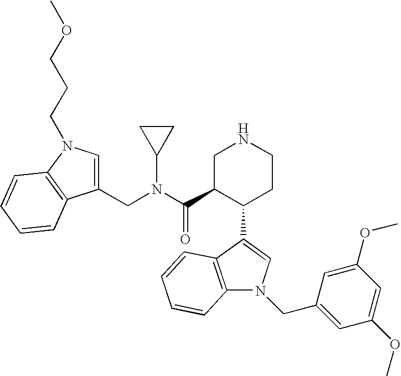

Example 3 is synthesized by deprotection of Intermediate 3.1 (114 mg, 0.16 mmol) analogously to the preparation of Example 1. White solid; ES-MS: M+H=635; HPLC: t_{Ret}=3.82 min.

### Intermediate 3.1:

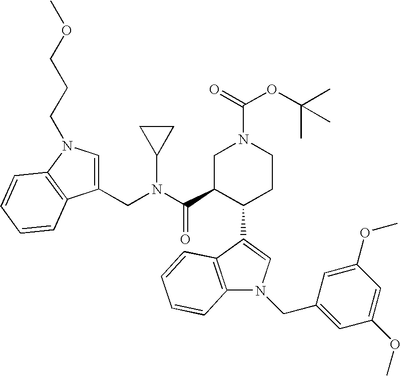

Intermediate 3.1 is synthesized by condensation of Intermediate 3.2 (152 mg, 0.31 mmol) and Intermediate 1.2 (95 mg, 0.37 mmol) analogously to the preparation of Intermediate 1.1. White amorphous material; ES-MS: M+H=735; HPLC: t_{Ret}=5.53 min.

### Intermediate 3.2:

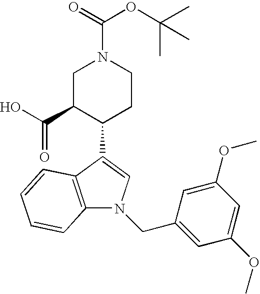

Intermediate 3.2 is synthesized by hydrolysis of Intermediate 3.3 (158 mg, 0.31 mmol) analogously to the preparation of Intermediate 1.4. White amorphous material; ES-MS: M-^{t}Bu=439; HPLC: t_{Ret}=4.49 min.

### Intermediate 3.3:

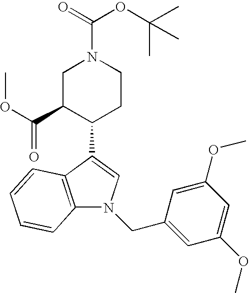

Intermediate 3.3 is synthesized by 1,4-reduction and epimerization of Intermediate 3.4 (164 mg, 0.32 mmol) analogously to the preparation of Intermediate 1.5. White solid; ES-MS: M-^{t}Bu=453; HPLC: t_{Ret}=5.05 min.

### Intermediate 3.4:

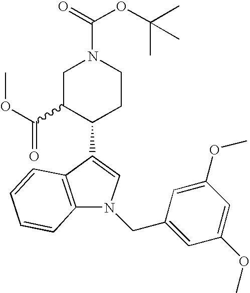

1,4-reduction of Intermediate 3.5 (250 mg, 0.51 mmol) analogously to the preparation of Intermediate 1.5. Then a mixture of crude material, 3,5-dimethoxybenzyl bromide (231 mg, 1.0 mmol) and NaH (30 mg, 0.75 mmol) in DMF (3 mL) is stirred at 0° C. for 2 h. After adding H₂O, the reaction mixture is extracted with EtOAc. The combined organic phases are washed with H₂O, brine and dried (MgSO₄). Concentration under reduced pressure and silica gel flash chromatography give Intermediate 3.4 as white amorphous material; ES-MS: M-^{t}Bu=453; HPLC: t_{Ret}=5.03 min.

### Intermediate 3.5:

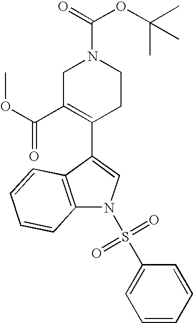

Intermediate 3.5 is synthesized by condensation of 4-trifluoromethanesulfonyloxy-5,6-dihydro-2H-pyridine-1,3-dicarboxylic acid 1-tert-butyl ester 3-methyl ester (1.08 g, 2.77 mmol) and 1-(phenylsulfonyl)-3-indoleboronic acid (1.0 g, 3.3 mmol) analogously to the preparation of Intermediate 1.6. Colorless solid; ES-MS: M-Boc=397; HPLC: t_{Ret}=4.75 min.

The following Examples enlisted on Table 1 are synthesized analogously to the preparation of Example 1-3. As far as not being commercially available, the synthesis of intermediates for the preparation of compounds of Examples 4-15 is described below the Table 1. The asterisk (*) marks the end of the bond via at which a moiety is bound to the rest of the molecule:

TABLE 1

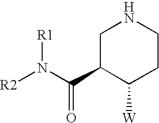

| Example | R1 | R2 | W | Analytical data |
|---|---|---|---|---|
| 4 | 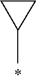 | 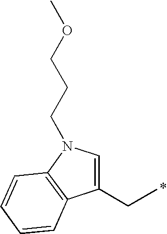 | 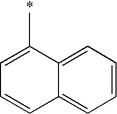 | MS: [M + 1]⁺ = 496 HPLC t_{Ret} = 3.59 min. |
| 5 | 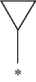 | 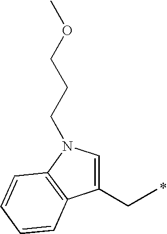 | 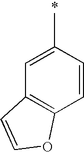 | MS: [M + 1]⁺ = 486 HPLC t_{Ret} = 3.30 min. |
| 6 | 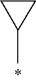 | 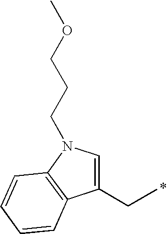 | 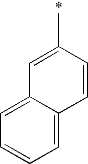 | MS: [M + 1]⁺ = 496 HPLC t_{Ret} = 3.55 min. |
| 7 | 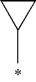 | 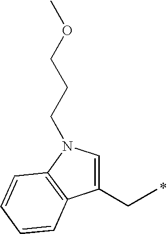 | 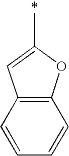 | MS: [M + 1]⁺ = 486 HPLC t_{Ret} = 3.54 min. |
| 8 | 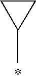 | 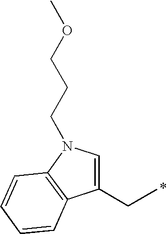 | 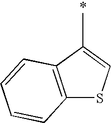 | MS: [M + 1]⁺ = 502 HPLC t_{Ret} = 3.59 min. |
| 9 | 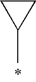 | 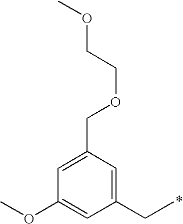 | 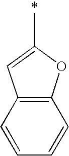 | MS: [M + 1]⁺ = 493 HPLC t_{Ret} = 3.25 min. |
| 10 | 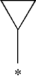 | 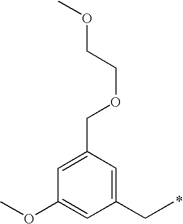 | 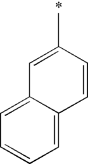 | MS: [M + 1]⁺ = 503 HPLC t_{Ret} = 3.23 min. |
| 11 | 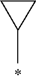 | 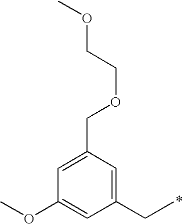 | 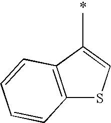 | MS: [M + 1]⁺ = 509 HPLC t_{Ret} = 3.25 min. |
| 12 | 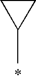 | 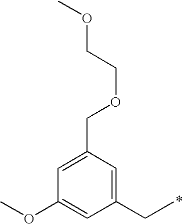 | 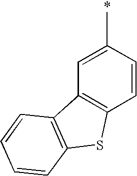 | MS: [M + 1]⁺ = 559 HPLC t_{Ret} = 3.50 min. |
| 13 | 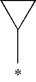 | 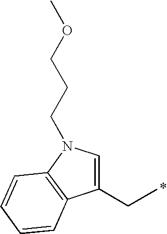 | 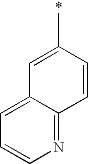 | MS: [M + 1]⁺ = 497 HPLC t_{Ret} = 2.10 min. |
| 14 | 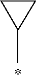 | 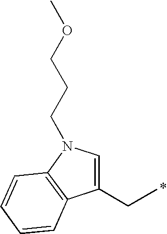 | 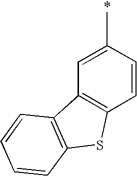 | MS: [M + 1]⁺ = 522 HPLC t_{Ret} = 3.85 min. |
| 15 | 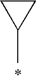 | 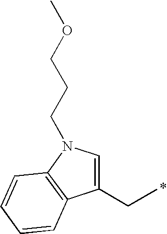 | 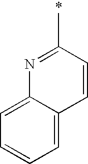 | |
| 16 | 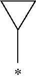 | 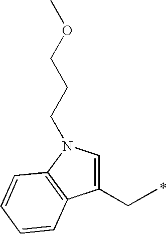 | 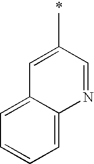 | MS: [M + 1]⁺ = 497 HPLC t_{Ret} = 2.38 min. |
| 17 | 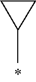 | 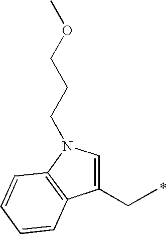 | 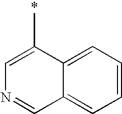 | |
| 18 | 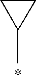 | 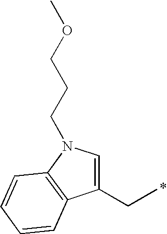 | 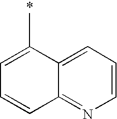 | MS: [M + 1]⁺ = 497 HPLC t_{Ret} = 2.22 min. |
| 19 | 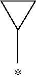 | 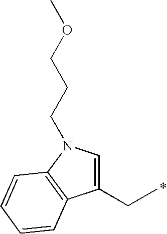 | 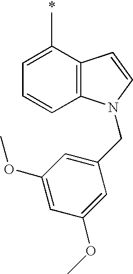 | MS: [M + 1]⁺ = 635 HPLC t_{Ret} = 3.55 min. |
| 20 | 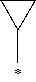 | 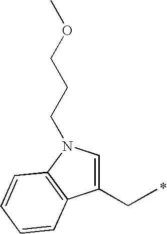 | 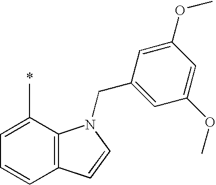 | Not synthesize |
| 21 | 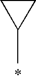 | 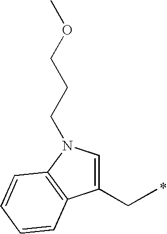 | 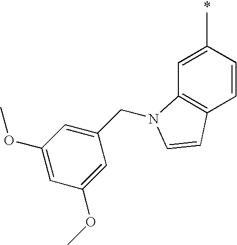 | MS: [M + 1]⁺ = 635 HPLC t_{Ret} = 3.65 min. |
| 22 | 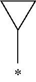 | 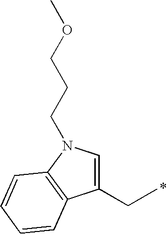 | 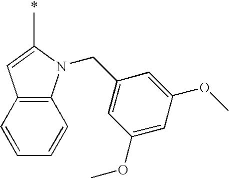 | Not synthesize |
| 23 | 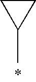 | 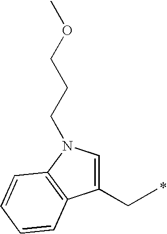 | 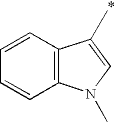 | MS: [M + 1]⁺ = 499 HPLC t_{Ret} = 3.25 min. |
| 24 | 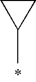 | 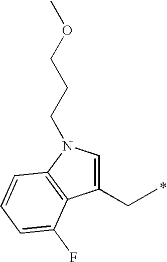 | 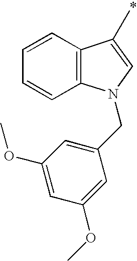 | MS: [M + 1]⁺ = 653 HPLC t_{Ret} = 3.75 min. |
| 25 | 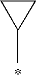 | 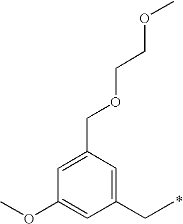 | 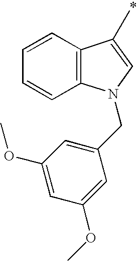 | MS: [M + 1]⁺ = 642 HPLC t_{Ret} = 3.40 min. |
| 26 | 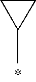 | 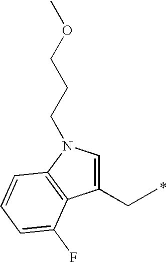 | 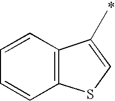 | MS: [M + 1]⁺ = 520 HPLC tRet = 3.40 min. |
| 27 | 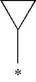 | 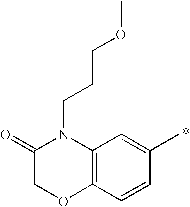 | 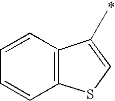 | MS: [M + 1]⁺ = 520 HPLC t_{Ret} = 2.93 min. |
| 28 | 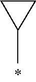 | 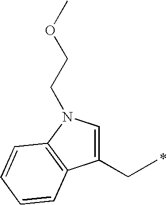 | 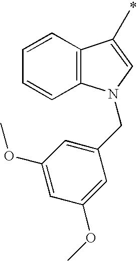 | MS: [M + 1]⁺ = 621 HPLC t_{Ret} = 3.59 min. |
| 29 | 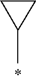 | 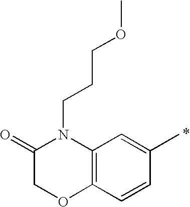 | 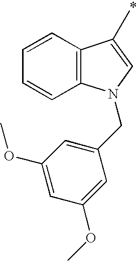 | MS: [M + 1]⁺ = 653 HPLC t_{Ret} = 3.23 min. |
| 30 | 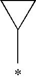 | 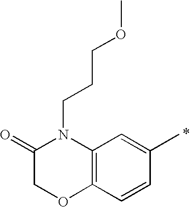 | 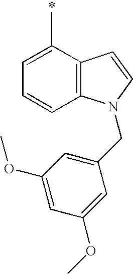 | MS: [M + 1]⁺ = 653 HPLC t_{Ret} = 3.22 min. |
| 31 | 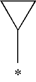 | 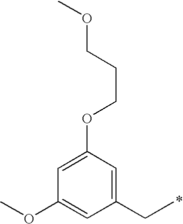 | 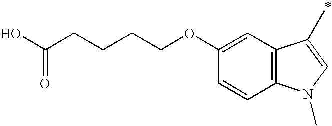 | MS: [M + 1]+ = 622 HPLC tRet = 3.01 min. |
| 32 | 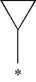 | 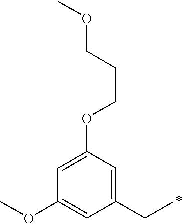 | 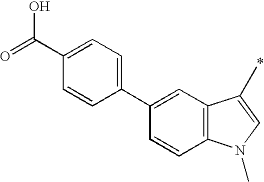 | MS: [M + 1]+ = 626 HPLC tRet = 3.12 min. |
| 33 | 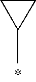 | 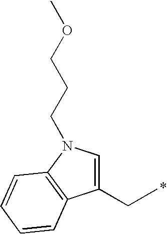 | 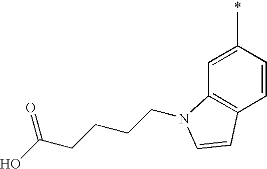 | MS: [M + 1]+ = 585 HPLC tRet = 3.09 min. |
| 34 | 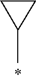 | 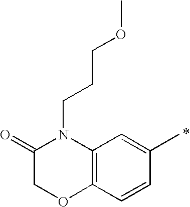 | 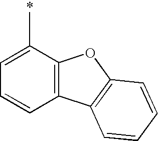 | MS: [M + 1]+ = 554 HPLC tRet = 2.92 min. |
| 35 | 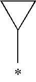 | 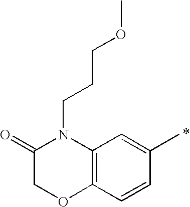 | 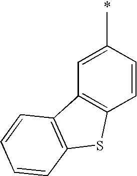 | MS: [M + 1]+ = 570 HPLC tRet = 3.10 min. |
| 36 | 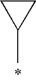 | 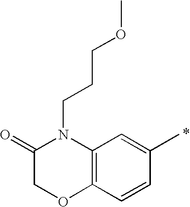 | 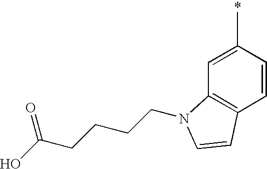 | MS: [M + 1]+ = 603 HPLC tRet = 2.75 min. |
| 37 | 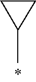 | 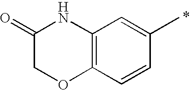 | 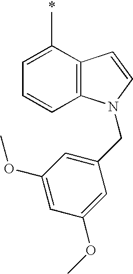 | MS: [M + 1]+ = 581 HPLC tRet = 2.95 min. |
| 38 | 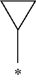 | 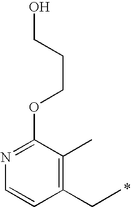 | 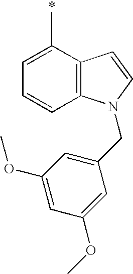 | MS: [M + 1]+ = 613 HPLC tRet = 2.52 min. |
| 39 | 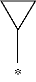 | 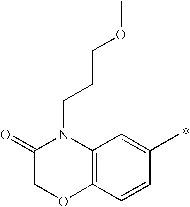 | 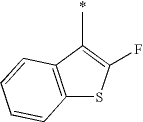 | MS: [M + 1]+ = 538 HPLC tRet = 2.65 min. |
| 40 | 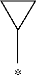 | 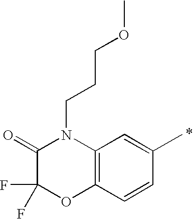 | 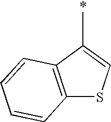 | MS: [M + 1]+ = 556 HPLC tRet = 3.05 min. |
| 41 | 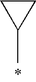 | 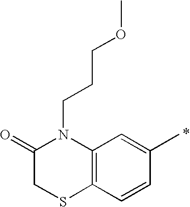 | 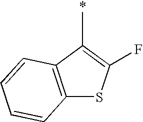 | MS: [M + 1]+ = 554 HPLC tRet = 2.85 min. |
| 42 | 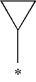 | 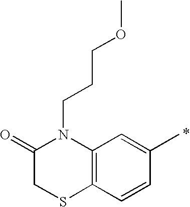 | 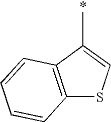 | MS: [M + 1]+ = 536 HPLC tRet = 2.80 min. |
| 43 | 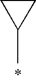 | 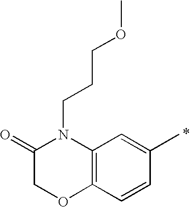 | 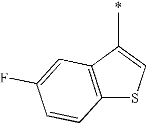 | MS: [M + 1]+ = 538 HPLC tRet = 2.73 min. |
| 44 (eut) | 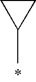 | 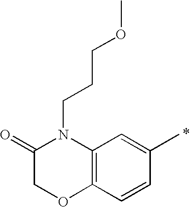 | 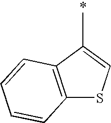 | MS: [M + 1]+ = 520 HPLC tRet = 2.67 min |
| 45 (dist) | 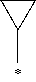 | 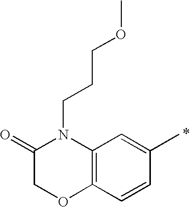 | 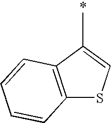 | MS: [M + 1]+ = 520 HPLC tRet = 2.63 min. |

### Intermediate 4.1:

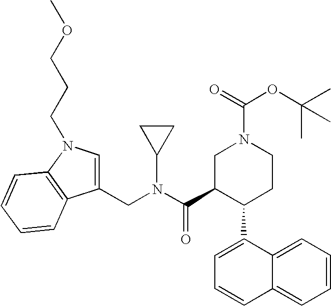

Intermediate 4.1 is synthesized by condensation of Intermediate 4.2 (210 mg, 0.59 mmol) and Intermediate 1.2 (198 mg, 0.77 mmol) analogously to the preparation of Intermediate 1.1. White solid; ES-MS: M+H=596; HPLC: t_{Ret}=5.49 min.

### Intermediate 4.2:

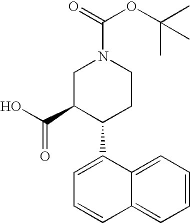

Intermediate 4.2 is synthesized by 1,4-reduction, epimerization and hydrolysis of Intermediate 4.3 (2.7 g, 7.34 mmol) analogously to the preparation of Intermediate 1.5 and 1.4. White solid; ES-MS: M-^{t}Bu=300; HPLC: t_{Ret}=4.20 min.

### Intermediate 4.3:

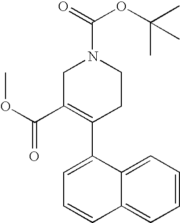

Intermediate 4.3 is synthesized by condensation of 4-trifluoromethanesulfonyloxy-5,6-dihydro-2H-pyridine-1,3-dicarboxylic acid 1-tert-butyl ester 3-methyl ester (3.01 g, 7.73 mmol) and 1-Naphthylboronic acid (1.61 g, 9.36 mmol) analogously to the preparation of Intermediate 1.6. Yellow solid; Rf=0.41 (AcOEt:n-Hex=1:3); ¹H NMR (CDCl₃) δ 1.52 (s, 9H), 2.42-2.62 (m, 2H), 3.29 (s, 3H), 3.52-3.60 (m, 1H), 3.69-3.83 (m, 1H), 4.33 (d, 1H), 4.45 (d, 1H), 7.11 (d, 1H), 7.40-7.49 (m, 3H), 7.65 (d, 1H), 7.77 (d, 1H), 7.83 (d, 1H).

### Intermediate 5.1:

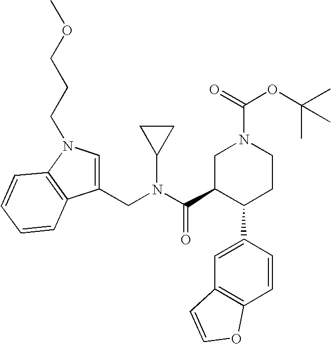

Intermediate 5.1 is synthesized by condensation of Intermediate 5.2 (210 mg, 0.61 mmol) and Intermediate 1.2 (204 mg, 0.79 mmol) analogously to the preparation of Intermediate 1.1. White solid; ES-MS: M+H=586; HPLC: t_{Ret}=5.12 min.

### Intermediate 5.2:

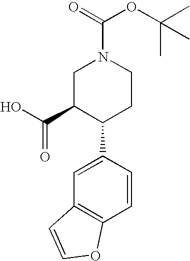

Intermediate 5.2 is synthesized by 1,4-reduction, epimerization and hydrolysis of Intermediate 5.3 (1.37 g, 3.85 mmol) analogously to the preparation of Intermediate 1.5 and 1.4. White solid; ES-MS: M-^{t}Bu=290; HPLC: t_{Ret}=3.93 min.

### Intermediate 5.3:

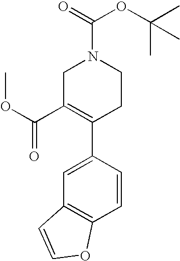

Intermediate 5.3 is synthesized by condensation of 4-trifluoromethanesulfonyloxy-5,6-dihydro-2H-pyridine-1,3-dicarboxylic acid 1-tert-butyl ester 3-methyl ester (1.5 g, 3.85 mmol) and 5-benzofuranylboronic acid (814 mg, 5.02 mmol) analogously to the preparation of Intermediate 1.6. Colorless solid; ES-MS: M-^{t}Bu=302; HPLC: t_{Ret}=4.52 min.

### Intermediate 6.1:

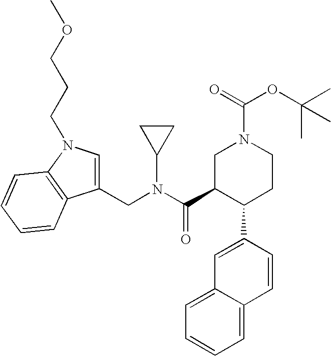

Intermediate 6.1 is synthesized by condensation of Intermediate 6.2 (210 mg, 0.59 mmol) and Intermediate 1.2 (198 mg, 0.77 mmol) analogously to the preparation of Intermediate 1.1. White solid; ES-MS: M+H=596; HPLC: t_{Ret}=5.47 min.

### Intermediate 6.2:

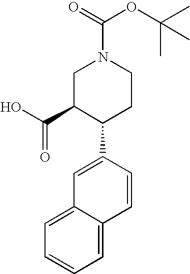

Intermediate 6.2 is synthesized by 1,4-reduction, epimerization and hydrolysis of Intermediate 6.3 (1.89 g, 5.13 mmol) analogously to the preparation of Intermediate 1.5 and 1.4. White solid; ES-MS: M-^{t}Bu=300; HPLC: t_{Ret}=4.24 min.

### Intermediate 6.3:

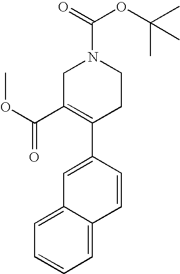

Intermediate 6.3 is synthesized by condensation of 4-trifluoromethanesulfonyloxy-5,6-dihydro-2H-pyridine-1,3-dicarboxylic acid 1-tert-butyl ester 3-methyl ester (2.0 g, 5.13 mmol) and 2-Naphthylboronic acid (1.14 g, 6.68 mmol) analogously to the preparation of Intermediate 1.6. Colorless solid; ES-MS: M-^{t}Bu=312; HPLC: t_{Ret}=4.92 min.

### Intermediate 7.1:

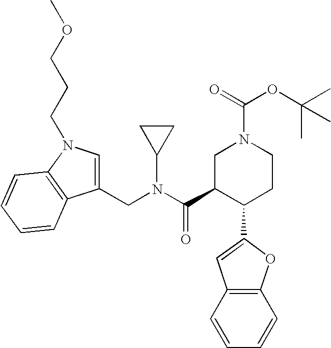

Intermediate 7.1 is synthesized by condensation of Intermediate 7.2 (208 mg, 0.60 mmol) and Intermediate 1.2 (202 mg, 0.78 mmol) analogously to the preparation of Intermediate 1.1. White amorphous material; ES-MS: M+H=586; HPLC: t_{Ret}=5.45 min.

### Intermediate 7.2:

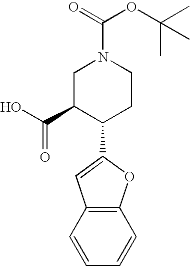

Intermediate 7.2 is synthesized by 1,4-reduction, epimerization and hydrolysis of Intermediate 7.3 (770 mg, 2.15 mmol) analogously to the preparation of Intermediate 1.5 and 1.4. White solid; ES-MS: M-^{t}Bu=290; HPLC: t_{Ret}=4.09 min.

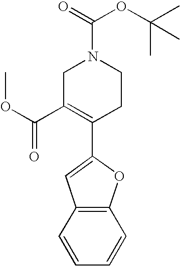

### Intermediate 7.3:

Intermediate 7.3 is synthesized by condensation of 4-trifluoromethanesulfonyloxy-5,6-dihydro-2H-pyridine-1,3-dicarboxylic acid 1-tert-butyl ester 3-methyl ester (2.0 g, 5.13 mmol) and 2-benzofuranylboronic acid (1.66 g, 10.3 mmol) analogously to the preparation of Intermediate 1.6. Colorless solid; ES-MS: M-^{t}Bu=290; HPLC: t_{Ret}=4.67 min.

### Intermediate 8.1:

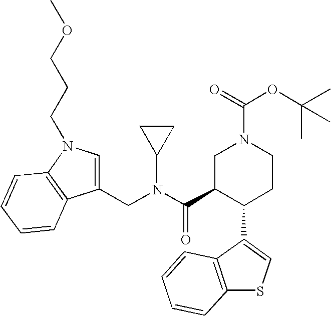

Intermediate 8.1 is synthesized by condensation of Intermediate 8.2 (200 mg, 0.55 mmol) and Intermediate 1.2 (186 mg, 0.72 mmol) analogously to the preparation of Intermediate 1.1. White solid; ES-MS: M+H=602; HPLC: t_{Ret}=5.42 min.

### Intermediate 8.2:

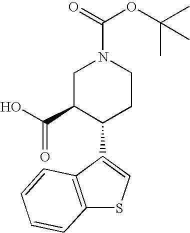

Intermediate 8.2 is synthesized by 1,4-reduction, epimerization and hydrolysis of Intermediate 8.3 (2.8 g, 7.5 mmol) analogously to the preparation of Intermediate 1.5 and 1.4. White solid; ES-MS: M-^{t}Bu=320; HPLC: t_{Ret}=4.74 min.

### Intermediate 8.3:

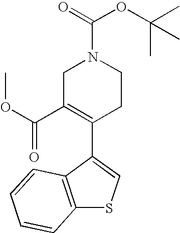

Intermediate 8.3 is synthesized by condensation of 4-trifluoromethanesulfonyloxy-5,6-dihydro-2H-pyridine-1,3-dicarboxylic acid 1-tert-butyl ester 3-methyl ester (3.07 g, 7.88 mmol) and benzothiophene-3-boronic acid (1.74 g, 9.77 mmol) analogously to the preparation of Intermediate 1.6. Yellow solid; Rf=0.38 (AcOEt:n-Hex=1:3); ¹H NMR (CDCl₃) δ 1.50 (s, 9H), 2.52-2.59 (m, 2H), 3.34 (s, 3H), 3.63-3.68 (m, 2H), 4.30-4.37 (m, 2H), 7.15 (s, 1H), 7.28-7.34 (m, 2H), 7.51-7.55 (m, 1H), 7.87-7.90 (m, 1H).

### Intermediate 9.1:

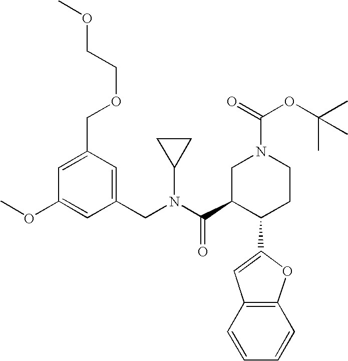

Intermediate 9.1 is synthesized by condensation of Intermediate 7.2 (210 mg, 0.61 mmol) and Intermediate 9.1 (210 mg, 0.79 mmol) analogously to the preparation of Intermediate 1.1. White solid; ES-MS: M+H=593; HPLC: t_{Ret}=5.09 min.

### Intermediate 9.1:

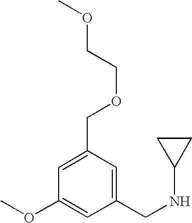

Intermediate 9.1 is synthesized by condensation of Intermediate 9.2 (10.3 g, 45.9 mmol) and cyclopropylamine (6.4 mL, 91.8 mmol) analogously to the preparation of Intermediate 1.2. Colorless oil; Rf=0.20 (AcOEt:DCM=2:1); ¹H NMR (CDCl₃) δ 0.33-0.45 (m, 4H), 2.12-2.18 (m, 1H), 3.39 (s, 3H), 3.54-3.63 (m, 4H), 3.79 (s, 3H), 4.54 (s, 2H), 6.75 (s, 1H), 6.77 (s, 1H), 6.85 (s, 1H).

### Intermediate 9.2:

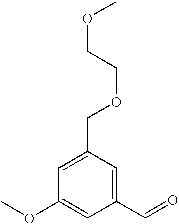

A mixture of Intermediate 9.3 (12.9 g, 57 mmol) and MnO₂ (17.5 g, excess) in EtOAc (200 mL) is stirred under N₂ at 60° C. for 4 h. After filtration removing MnO₂, the filtrate is concentrated under reduced pressure and silica gel flash chromatography to give Intermediate 9.2 as colorless oil; Rf=0.45 (AcOEt:n-Hex=1:1); ¹H NMR (CDCl₃) δ 3.39 (s, 3H), 3.56-3.68 (m, 4H), 3.87 (s, 3H), 4.61 (s, 2H), 7.19 (s, 1H), 7.30 (s, 1H), 7.47 (s, 1H), 9.98 (s, 1H).

### Intermediate 9.3:

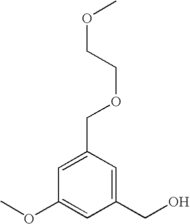

A mixture of Intermediate 9.4 (824 mg, 3.3 mmol) and LAH (174 mg, 6.6 mmol) in THF (12 mL) is stirred under N₂ at 0° C. for 3 h. After adding H₂O, the reaction mixture is extracted with EtOAc. The combined organic phases are washed with H₂O, brine and dried (Na₂SO₄). Concentration under reduced pressure and silica gel flash chromatography give Intermediate 9.3. White powder; HPLC: _{A}t_{Ret}=2.52 min; Rf=0.21 (EtOAc:n-Hex=1:1).

### Intermediate 9.4:

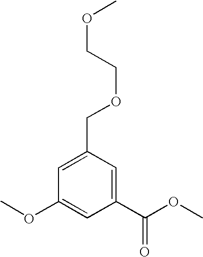

Intermediate 9.4 is synthesized by alkylation of 3-(hydroxymethyl)-5-methoxy-benzoic acid methylester (1.85 g, 9.4 mmol) (see e.g. Synthetic Communications, 2001, 31, 1921-1926) analogously to the preparation of Intermediate 1.3. White amorphous material; ES-MS: M+H=255; HPLC: _{A}t_{Ret}=3.44 min

### Intermediate 10.1:

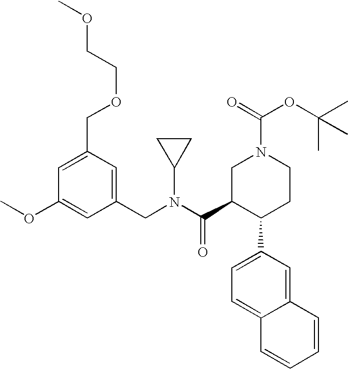

Intermediate 10.1 is synthesized by condensation of Intermediate 6.2 (205 mg, 0.58 mmol) and Intermediate 9.1 (199 mg, 0.75 mmol) analogously to the preparation of Intermediate 1.1. Colorless oil; ES-MS: M+H=603; HPLC: t_{Ret}=5.03 min.

### Intermediate 11.1:

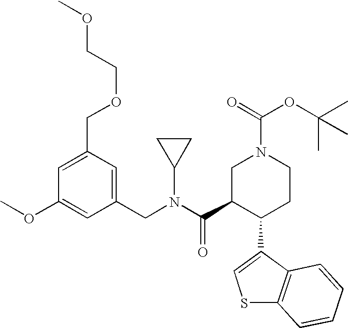

Intermediate 11.1 is synthesized by condensation of Intermediate 8.2 (205 mg, 0.57 mmol) and Intermediate 9.1 (196 mg, 0.74 mmol) analogously to the preparation of Intermediate 1.1. Colorless oil; ES-MS: M+H=609; HPLC: t_{Ret}=5.10 min.

### Intermediate 12.1:

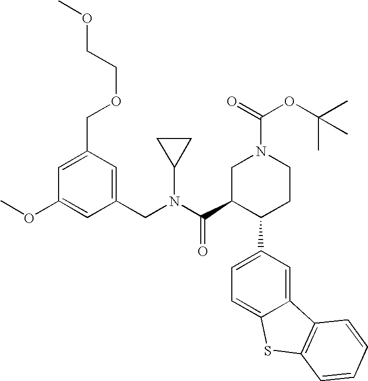

Intermediate 12.1 is synthesized by condensation of Intermediate 12.2 (210 mg, 0.51 mmol) and Intermediate 9.1 (176 mg, 0.66 mmol) analogously to the preparation of Intermediate 1.1. White solid; ES-MS: M+H=659; HPLC: t_{Ret}=5.49 min.

### Intermediate 12.2:

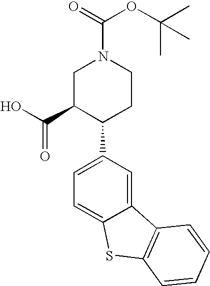

Intermediate 12.2 is synthesized by 1,4-reduction, epimerization and hydrolysis of Intermediate 12.3 (1.78 g, 4.2 mmol) analogously to the preparation of Intermediate 1.5 and 1.4. White solid; ES-MS: M-^{t}Bu=356; HPLC: t_{Ret}=4.64 min.

### Intermediate 12.3:

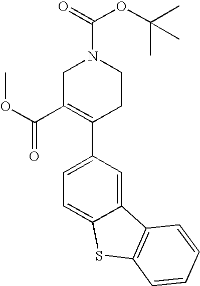

Intermediate 12.3 is synthesized by condensation of 4-trifluoromethanesulfonyloxy-5,6-dihydro-2H-pyridine-1,3-dicarboxylic acid 1-tert-butyl ester 3-methyl ester (1.66 g, 4.27 mmol) and 2-dibenzothienylboronic acid (0.99 g, 4.34 mmol) analogously to the preparation of Intermediate 1.6. Yellow solid; Rf=0.40 (AcOEt:n-Hex=1:3); ¹H NMR (CDCl₃) δ 1.52 (s, 9H), 2.58-2.61 (m, 2H), 3.47 (s, 3H), 3.67 (t, 2H), 4.70 (brs, 2H), 7.20 (d, 1H), 7.44-7.49 (m, 2H), 7.70 (d, 1H), 7.71-7.73 (m, 1H), 7.91 (s, 1H), 8.11-8.14 (m, 1H).

### Intermediate 13.1:

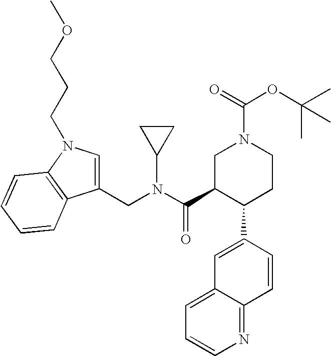

Intermediate 13.1 is synthesized by condensation of Intermediate 13.2 (296 mg, 0.83 mmol) and Intermediate 1.2 (258 mg, 1.00 mmol) analogously to the preparation of Intermediate 1.1. White solid; ES-MS: M+H=597; HPLC: t_{Ret}=3.53 min.

### Intermediate 13.2:

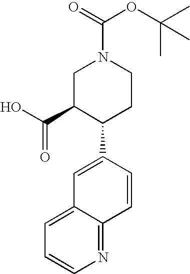

Intermediate 13.2 is synthesized by epimerization and hydrolysis of Intermediate 13.3 (398 mg, 1.07 mmol) analogously to the preparation of Intermediate 1.5 and 1.4. White amorphous material; ES-MS: M+H=357; HPLC: t_{Ret}=2.72 min.

### Intermediate 13.3:

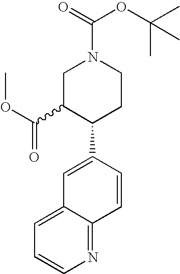

Intermediate 13.3 is synthesized by oxidation of Intermediate 13.4 (524 mg, 1.41 mmol) analogously to the preparation of Intermediate 9.2. Colorless amorphous material; ES-MS: M+H=371; HPLC: t_{Ret}=2.82 and 2.92 min.

### Intermediate 13.4:

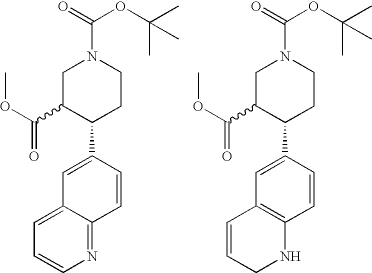

Intermediate 13.4 is synthesized by 1,4-reduction of Intermediate 13.5 (1.67 g, 4.53 mmol) analogously to the preparation of Intermediate 1.5. Colorless oil; ES-MS: M+H=371 and M+=373; HPLC: t_{Ret}=2.81 and 2.91 min. Both compounds are used in the next reaction step as mixture.

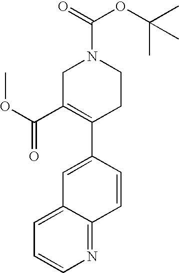

### Intermediate 13.5:

Intermediate 13.5 is synthesized by condensation of 4-trifluoromethanesulfonyloxy-5,6-dihydro-2H-pyridine-1,3-dicarboxylic acid 1-tert-butyl ester 3-methyl ester (1.77 g, 4.54 mmol) and quinoline-6-boronic acid (943 mg, 5.45 mmol) analogously to the preparation of Intermediate 1.6. Colorless solid; ES-MS: M+H=369; HPLC: t_{Ret}=2.80 min.

### Intermediate 14.1:

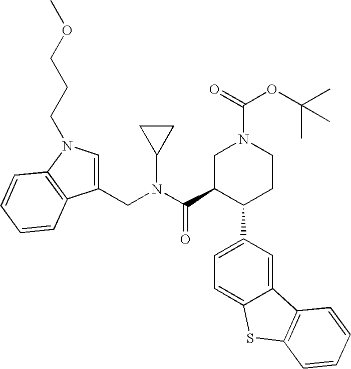

Intermediate 14.1 is synthesized by condensation of Intermediate 12.2 (205 mg, 0.498 mmol) and Intermediate 1.2 (167 mg, 0.648 mmol) analogously to the preparation of Intermediate 1.1 White solid; ES-MS: M−H=650; HPLC: t_{Ret}=5.80 min.

### Intermediate 16.1:

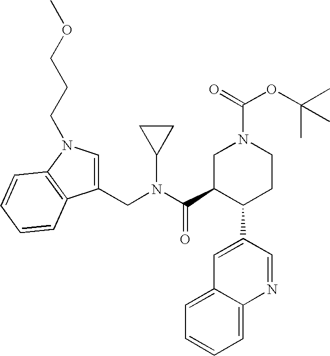

Intermediate 16.1 is synthesized by condensation of Intermediate 16.2 (202 mg, 0.57 mmol) and Intermediate 1.2 (176 mg, 0.68 mmol) analogously to the preparation of Intermediate 1.1 White solid; ES-MS: M+H=597; HPLC: t_{Ret}=3.45 min.

### Intermediate 16.2:

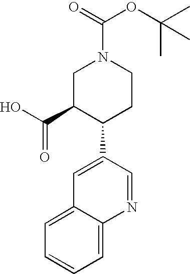

Intermediate 16.2 is synthesized by epimerization and hydrolysis of Intermediate 16.3 (2.32 g, 6.26 mmol) analogously to the preparation of Intermediate 1.5 and 1.4. White solid; ES-MS: M+H=357; HPLC: t_{Ret}=2.57 min.

### Intermediate 16.3:

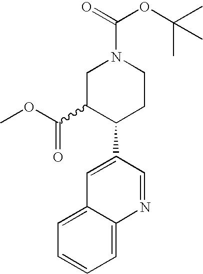

Intermediate 16.3 is synthesized by 1,4-reduction of Intermediate 16.4 (2.32 g, 6.30 mmol) analogously to the preparation of Intermediate 1.5. White solid; ES-MS: M+H=371; HPLC: t_{Ret}=2.82 min.

### Intermediate 16.4:

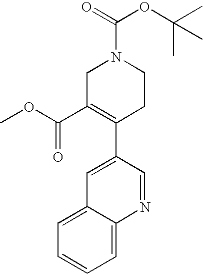

Intermediate 16.4 is synthesized by condensation of 4-trifluoromethanesulfonyloxy-5,6-dihydro-2H-pyridine-1,3-dicarboxylic acid 1-tert-butyl ester 3-methyl ester (7.73 g, 19.9 mmol) and quinoline-2-boronic acid (4.27 g, 24.7 mmol) analogously to the preparation of Intermediate 1.6. Colorless solid; ES-MS: M+H=369; HPLC: t_{Ret}=2.90 min.

### Intermediate 18.1:

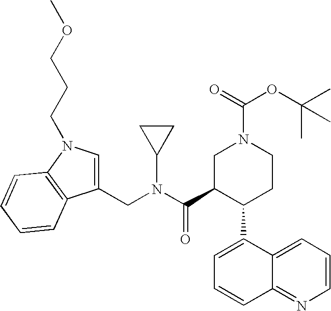

Intermediate 18.1 is synthesized by 1,4-reduction, epimerization, hydrolysis and condensation of Intermediate 18.2 (1.7 g, 4.6 mmol) analogously to the preparation of Intermediate 1.5, 1.4, and 1.1 White solid; ES-MS: M+H=597; HPLC: t_{Ret}=3.38 min.

### Intermediate 18.2:

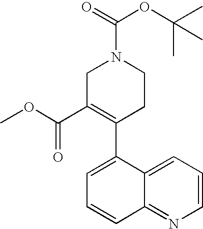

Intermediate 18.2 is synthesized by condensation of 4-trifluoromethanesulfonyloxy-5,6-dihydro-2H-pyridine-1,3-dicarboxylic acid 1-tert-butyl ester 3-methyl ester (1.92 g, 4.92 mmol) and quinoline-4-boronic acid (1.02 g, 5.90 mmol) analogously to the preparation of Intermediate 1.6. Colorless oil; ES-MS: M+H=369; HPLC: t_{Ret}=2.79 min.

### Intermediate 19.1:

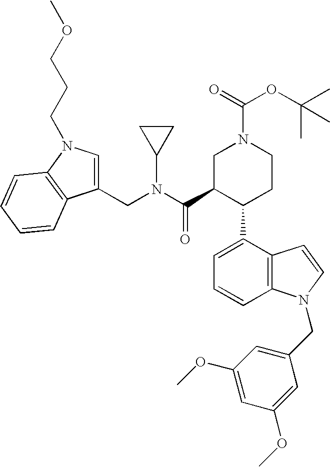

Intermediate 19.1 is synthesized by condensation of Intermediate 19.2 (210 mg, 0.42 mmol) and Intermediate 1.2 (121 mg, 0.46 mmol) analogously to the preparation of Intermediate 1.1. Colorless oil; ES-MS: M+H=735; HPLC: t_{Ret}=5.49 min.

### Intermediate 19.2:

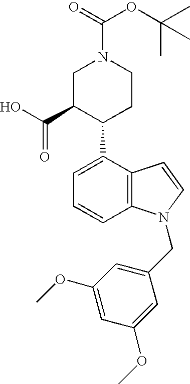

Intermediate 19.2 is synthesized by 1,4-reduction, epimerization and hydrolysis of Intermediate 19.3 (2.59 g, 5.11 mmol) analogously to the preparation of Intermediate 1.4 and 1.5. Colorless oil; ES-MS: M+H-Bu^{t}=439; HPLC: t_{Ret}=4.37 min.

### Intermediate 19.3:

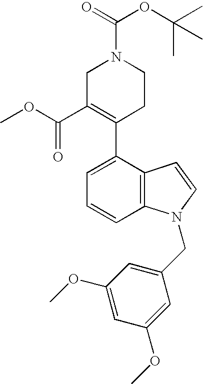

Intermediate 19.3 is synthesized by condensation of 4-trifluoromethanesulfonyloxy-5,6-dihydro-2H-pyridine-1,3-dicarboxylic acid 1-tert-butyl ester 3-methyl ester (2.67 g, 5.27 mmol) and Intermediate 19.4 (2.08 g, 5.28 mmol) analogously to the preparation of Intermediate 1.6. Orange gum; Rf 0.46 (n-hexane/EtOAc=7:4), ¹H-NMR: 1.52 (s, 9H), 2.60-2.67 (m, 2H), 3.30 (s, 3H), 3.62-3.69 (m, 2H), 3.70 (s, 6H), 4.28-4.33 (m, 2H), 5.23 (s, 2H), 6.20 (d, 2H, J=1.0), 6.28-6.31 (m, 2H), 6.83 (d, J=8.0), 7.10 (d, J=3.0), 7.15 (t, J=8.0), 7.21 (J=8.0).

### Intermediate 19.4:

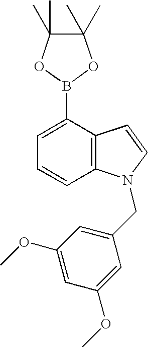

To a deoxygenated solution of Intermediate 19.5 (2.65 g, 7.65 mmol) and bis(pinacol)diborate (2.0 g, 8.42 mmol) in DMSO (40 mL), KOAc (2.34 g, 23.0 mmol) and PdCl₂(dppf) (0.69 g, 0.77 mmol) are added under N₂ at RT. After stirring at 80 C for 24 h, the mixture is treated with water and filtered through a Celite pad. The filtrate is extracted with EtOAc and ether. The combined organic extracts are washed with water, dried over Na₂SO₄, filtered, and concentrated. Silica gel column chromatography affords Intermediate 19.4 as light green crystals: Rf 0.42 (n-hexane/EtOAc=3:1), ¹H-NMR: 1.39 (s, 12H), 3.70 (s, 6H), 5.26 (s, 2H), 6.27 (d, 2H, J=1.0), 6.33 (t, J=1.0), 7.04 (d, J=3.0), 7.16-7.20 (m, 2H), 7.38 (d, J=9.0), 7.62 (d, J=9.0).

### Intermediate 19.5:

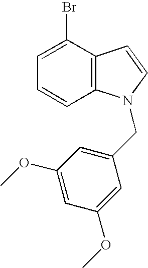

To a solution of 4-bromoindole (1.74 g, 8.87 mmol) in DMF (26 mL), NaH (60%, 485 mg, 10.6 mmol) and 3,5-dimethoxybenzyl bromide (2.54 g, 10.6 mmol) are added at 0 C. After stirring at 0° C. to RT for 1 h, the mixture is poured into water. After extraction with EtOAc, the combined organic layer is washed with water, dried over Na2SO4, filtered, and concentrated. Silica gel column chromatography affords Intermediate 19.5 as a yellow oil: Rf 0.46 (n-hexane/EtOAc=3:1), 1H-NMR: 3.72 (s, 6H), 5.24 (s, 2H), 6.22 (d, 2H, J=1.0), 6.36 (t, J=1.0), 6.59 (d, J=3.0), 7.01 (t, J=9.0), 7.17 (d, J=3.0), 7.24 (d, J=9.0), 7.27 (d, J=9.0).

### Intermediate 21.1:

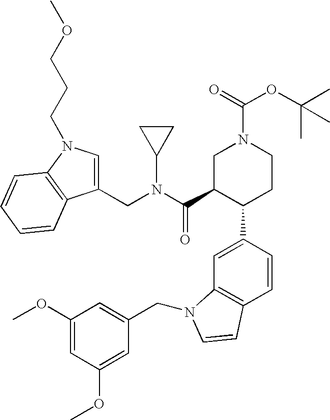

Intermediate 21.1 is synthesized by condensation of Intermediate 21.2 (150 mg, 0.3 mmol) and Intermediate 1.2 (94 mg, 0.36 mmol) analogously to the preparation of Intermediate 1.1. Colorless oil; ES-MS: M+H=735; HPLC: t_{Ret}=5.50 min.

### Intermediate 21.2:

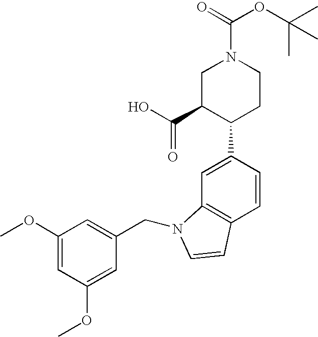

Intermediate 21.2 is synthesized by 1,4-reduction, epimerization and hydrolysis of Intermediate 21.3 (1 g, 2.0 mmol) analogously to the preparation of Intermediate 1.4 and 1.5. Colorless oil; ES-MS: M+H=495; HPLC: t_{Ret}=4.40 min.

### Intermediate 21.3:

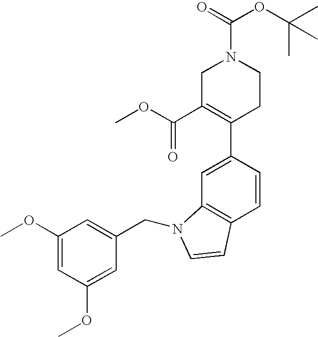

Intermediate 21.3 is synthesized by condensation of 4-trifluoromethanesulfonyloxy-5,6-dihydro-2H-pyridine-1,3-dicarboxylic acid 1-tert-butyl ester 3-methyl ester (6.31 g, 16.2 mmol) and Intermediate 21.4 (6.37 g, 16.2 mmol) analogously to the preparation of Intermediate 1.6. Light yellow solid; Rf 0.44 (n-hexane/EtOAc=7:4), ¹H-NMR: 1.50 (s, 9H), 2.49-2.56 (m, 2H), 3.30 (s, 3H), 3.59-3.63 (m, 2H), 3.72 (s, 6H), 4.20-4.28 (m, 2H), 5.20 (s, 2H), 6.21 (d, 2H, J=1.0), 6.32 (t, J=1.0), 6.51 (d, J=3.0), 6.89 (d, J=9.0), 7.06 (s), 7.11 (d, J=3.0), 7.55 (d, J=9.0).

### Intermediate 21.4:

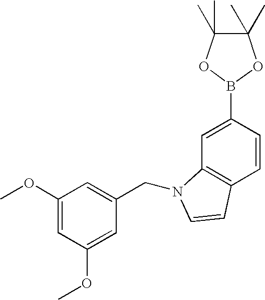

Intermediate 21.4 is synthesized by cross-coupling of Intermediate 21.5 (6.81 g, 19.67 mmol) analogously to the preparation of Intermediate 19.4. Light yellow solid; Rf 0.47 (nhexane/EtOAc=3:1), ¹H-NMR: 1.35 (s, 12H), 3.70 (s, 6H), 5.29 (s, 2H), 6.24 (d, J=1.0), 6.35 (t, J=1.0), 6.54 (d, J=3.0), 7.14 (d, J=3.0), 7.57 (d, J=9.0), 7.63 (d, J=9.0), 7.87 (s).

### Intermediate 21.5:

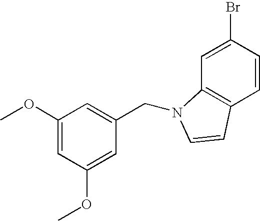

Intermediat 21.5 is synthesized by N-benzylation of 5-bromo-indole (4.20 g, 21.42 mmol) analogously to the preparation of Intermediate 19.5. White crystals: Rf 0.44 (nhexane/EtOAc=7:2), ¹H-NMR: 3.71 (s, 6H), 5.18 (s, 2H), 6.22 (d, 2H, J=1.0), 6.36 (t, J=1.0), 6.50 (d, J=3.0), 7.09 (t, J=3.0), 7.18 (d, J=9.0), 7.43 (s), 7.48 (d, J=9.0).

### Intermediate 23.1:

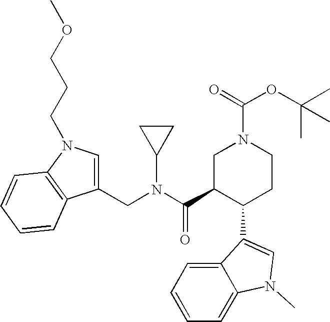

Intermediate 23.1 is synthesized by condensation of Intermediate 23.2 (59 mg, 0.17-mmol) and Intermediate 1.2 (51 mg, 0.2 mmol) analogously to the preparation of Intermediate 1.1. Colorless oil; ES-MS: M+H=599; HPLC: t_{Ret}=5.20 min.

### Intermediate 23.2:

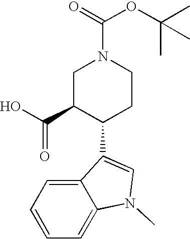

Intermediate 23.2 is synthesized by 1,4-reduction, epimerization and hydrolysis of Intermediate 23.3 (3.4 g, 9.2 mmol) analogously to the preparation of Intermediate 1.4 and 1.5. Colorless oil; ES-MS: M+H=359; HPLC: t_{Ret}=3.47 min.

### Intermediate 23.3:

To a solution of N-methyl indole (2.4 g, 18 mmol) in ACOH (40 mL), 4-oxo-piperidine-3-carboxylic acid methyl ester (6.7 g, 35 mmol) and 2NH₃PO₄ (10 mL) are added. After stirring at 110° C. for 17 h, the reaction micture is azeotropically concentrated with toluene. The reside is treated with Boc₂O (15.7 g, 72 mmol) in CH₂Cl₂ (200 mL) and sat. aq. NaHCO₃ (100 mL). After stirred at RT for 19 h, the mixture is diluted with water and extracted with CH₂Cl₂. The combined organic layer is washed with water and brine, dried over Na₂SO₄, filtered, and concentrated. Silica gel column chromatography affords Intermediate 23.3 as a yellow oil: ES-MS: M+H=371; HPLC: t_{Ret}=4.43 min.

### Intermediate 24.1:

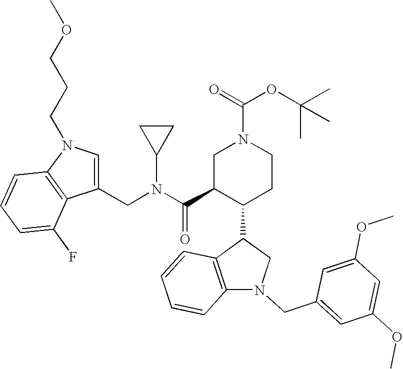

Intermediate 24.1 is synthesized by condensation of Intermediate 3.2 (143 mg, 0.29 mmol) and Intermediate 24.2 (96 mg, 0.35 mmol) analogously to the preparation of Intermediate 1.1. Colorless oil; ES-MS: M+H=753; HPLC: t_{Ret}=5.60 min.

### Intermediate 24.2:

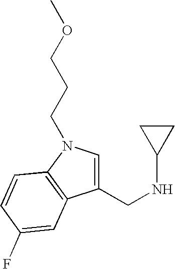

Intermediate 24.2 is synthesized by condensation of Intermediate 24.3 (640 mg, 2.70 mmol) and cyclopropylamine (308 mg, 5.40 mmol) analogously to the preparation of Intermediate 1.2. Colorless oil; ES-MS: M+H=277; HPLC: _{A}t_{Ret}=2.57 min.

### Intermediate 24.3:

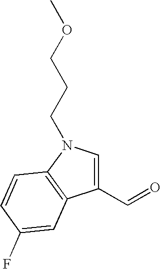

Intermediate 24.3 is synthesized by condensation of 5-fluoro-indole-3-carbaldehyde (500 mg, 3.10 mmol) and toluene-4-sulfonic acid 3-methoxy-propyl ester (973 mg, 3.90 mmol) analogously to the preparation of Intermediate 1.3. Yellow oil; ES-MS: M+H=236; HPLC: _{A}t_{Ret}=3.22 min.

### Intermediate 25.1:

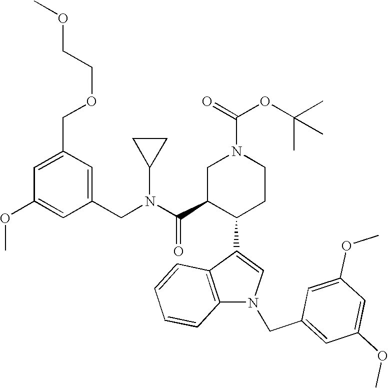

Intermediate 25.1 is synthesized by condensation of Intermediate 3.2 (132 mg, 0.27 mmol) and Intermediate 9.1 (78 mg, 0.29 mmol) analogously to the preparation of Intermediate 1.1. Colorless oil; ES-MS: M+H=742; HPLC: t_{Ret}=5.14 min.

### Intermediate 26.1:

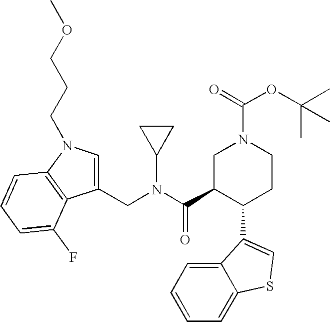

Intermediate 26.1 is synthesized by condensation of Intermediate 8.2 (154 mg, 0.43 mmol) and Intermediate 24.2 (141 mg, 0.51 mmol) analogously to the preparation of Intermediate 1.1. Colorless oil; ES-MS: M+H=620; HPLC: t_{Ret}=5.50 min.

### Intermediate 27.1:

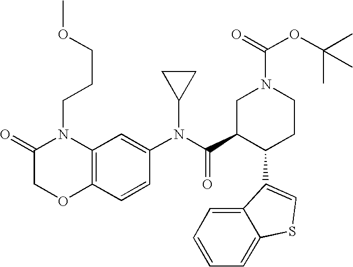

A mixture of Intermediate 8.2 (110 mg, 0.31 mmol) and 1-chloro-N,N-2-trimethyl-1-propenylamine (49 μL, 0.37 mmol) in DCM (1 mL) is stirred at RT. After stirring for 30 min, a mixture of Intermediate 27.2 (84 mg, 0.31 mmol) and pyridine (32 μL, 0.40 mmol) in DCM (1.5 mL) is added to the reaction mixture, and stirred for 3 h at RT. H₂O and CH₂Cl₂ are added and the organic phases is dried over Na₂SO₄ and evaporated in vacuo. Silica gel flash chromatography of the residue (hexane/ethyl acetate) affords Intermediate 27.1 as a colorless oil; ES-MS: M+H=620; HPLC: t_{Ret}=4.70 min.

### Intermediate 27.2

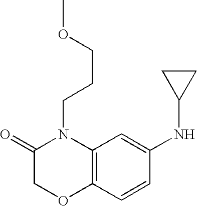

To a mixture of Intermediate 27.3 (680 mg, 3.3 mmol) in DMF (7 mL), NaH (160 mg, 4.0 mmol) is added under N₂ at 0° C. After stirring at RT for 30 min, toluene-4-sulfonic acid 3-methoxy-propyl ester (1.2 g, 4.9 mmol) and KI (99 mg, 0.6 mmol) are added to the reaction mixture at RT. After stirring at 60° C. for 2 h, H₂O is added to the reaction mixture which is then extracted with EtOAc. The combined organic phases are washed with H₂O and evaporated in vacuo. Silica gel flash chromatography give Intermediate 27.2 as pale yellow solid oil; ES-MS: M+H=277, HPLC: t_{Ret}=2.67 min.

### Intermediate 27.3

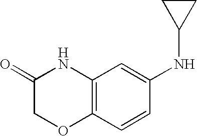

To a mixture of Intermediate 27.4 (1.6 g, 6.0 mmol), Zn (2.7 g, 42 mmol) and saturated aqueous NH₄Cl (20 mL) in EtOH (80 mL) are added at RT. After refluxing for 1 h, the reaction mixture is cooled down to RT, and is filtered Celite pad and diluted with EtOAc. The organic phases are washed with H₂O, brine and dried and evaporated in vacuo. The residue is suspended with EtOAc, and filtered through Celite. The filtrate is dried, and evaporated in vacuo to afford Intermediate 27.3 as pale red solid; ES-MS: M+H=205, HPLC: t_{Ret}=1.97 min.

### Intermediate 27.4

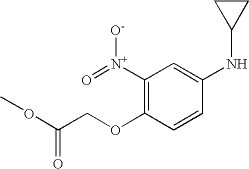

A mixture of Intermediate 27.5 (4 g, 20.6 mmol), Methyl bromoacetate (4.7 g, 30.8 mmol) and K₂CO₃ (4.8 g, 35.0 mmol) in CH₃CN (20 mL) is stirred for 24 h at RT. H₂O and EtOAc are added and the organic layer is washed with brine, dried over MgSO₄ and evaporated in vacuo. Silica gel flash chromatography of the residue (hexane/ethyl acetate) affords Intermediate 27.4 as orange oil material; ES-MS: M+H=264; HPLC: t_{Ret}=3.48 min.

### Intermediate 27.5

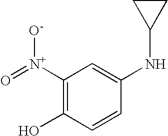

(1-Ethoxycyclopropoxy)trimethylsilane (3.9 g, 23.4 mmol) is slowly added to a mixture of 4-amino-2-nitrophenol (3 g, 19.5 mmol) in AcOH (80 mL) and MeOH (20 mL), and the resulting mixture is heated at 70° C. for 3 h. The reaction mixture is cooled at RT, and evaporated in vacuo, the residue is used further. BF₃-EtO₂ (3 mL, 23.4 mmol) is added to NaBH₄ (889 mg, 23.4 mmol) in THF (20 mL) dropwise at 0° C. Then, the residue in THF (80 mL) is added dropwise at 40° C. over 30 min. After stirring at 0° C. for 2 h, the reaction mixture is quenched with cold water and extracted with EtOAc. The combined organic phases are washed H₂O, brine and dried. Concentration under reduce pressure and Silica gel flash chromatography give Intermediate 27.5 as red solid; ES-MS: M+H=195PLC: t_{Ret}=3.47.

### Intermediate 28.1:

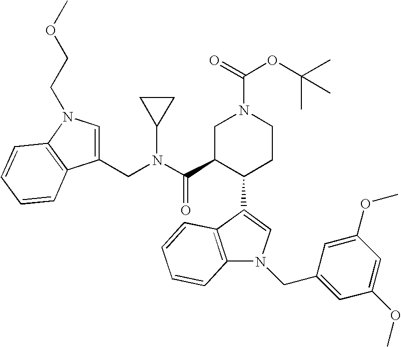

Intermediate 28.1 is synthesized from Intermediate 3.2 (135 mg, 0.27 mmol) analogously to the preparation of Intermediate 1.1. Colorless oil; ES-MS: M+H=721; HPLC: t_{Ret}=5.37 min.

### Intermediate 29.1:

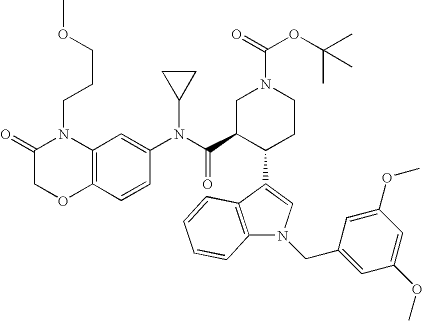

Intermediate 29.1 is synthesized by condensation of Intermediate 3.2 (156 mg, 0.32 mmol) and Intermediate 27.2 (87 mg, 0.32 mmol) analogously to the preparation of Intermediate 27.1. Colorless oil; ES-MS: M+H=753; HPLC: t_{Ret}=4.82 min.

### Intermediate 30.1:

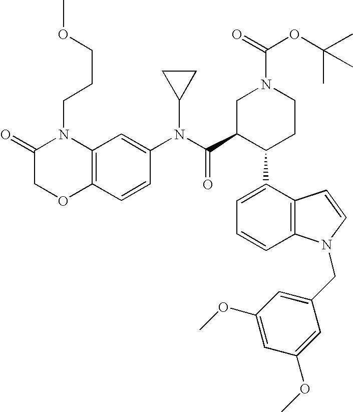

Intermediate 30.1 is synthesized by condensation of Intermediate 19.2 (152 mg, 0.31 mmol) and Intermediate 27.2 (85 mg, 0.31 mmol) analogously to the preparation of Intermediate 27.1. Colorless oil; ES-MS: M+H=753; HPLC: t_{Ret}=4.84 min.

### Intermediate 31.1

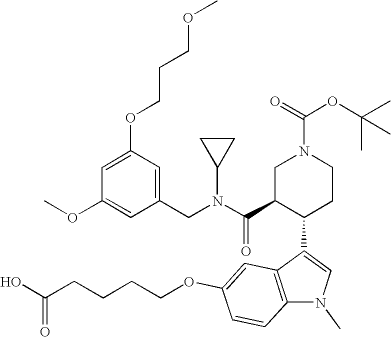

To a solution of Intermediate 31.2 (90 mg, 0.122 mmol) in MeOH is added aqueous NaOH (1M, 0.5 mL, 0.5 mmol) at room temperature, and the mixture is stirred at 60° C. After stirring for 4 h at 60° C., the reaction mixture is concentrated under reduced pressure. RP-HPLC purification affords Intermediate 31.1 as white amorphous; ES-MS: M+H=722; HPLC: _{A}t_{Ret}=4.39 min.

### Intermediate 31.2

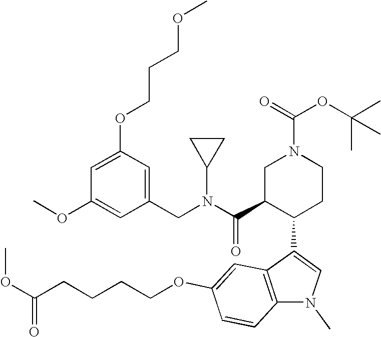

To a solution of Intermediate 31.3 (120 mg, 0.193 mmol) in DMF are added Cs₂CO₃ and 5-bromopentanoic acid methyl ester (57 mg, 0.29 mmol) at room temperature, and the resulting mixture is stirred at 60° C. After stirring for 16 h at 60° C., the reaction is quenched by addition of H₂O and the mixture is extracted with Et₂O. The organic layer is washed with H₂O and brine, then dried over Na₂SO₄ and concentrated under reduced pressure. R^{P}-HPLC purification affords Intermediate 31.2 as a yellow amorphous; ES-MS: M+H=736; HPLC: _{A}t_{Ret}=5.02 min.

### Intermediate 31.3

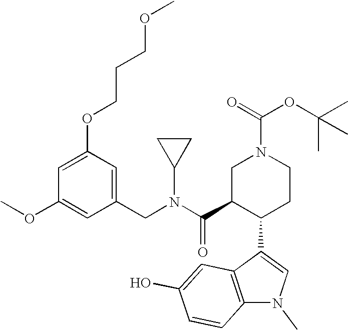

Intermediate 31.3 is synthesized by condensation of Intermediate 31.4 (400 mg, 1.07 mmol) with cyclopropyl[3-methoxy 5-(3-methoxypropoxy)benzyl]amine (340 mg, 1.28 mmol) analogously to the preparation of Intermediate 1.1 Yellow amorphous material; ES-MS: [M+H=622; HPLC: t_{Ret}=4.22 minutes.

### Intermediate 31.4

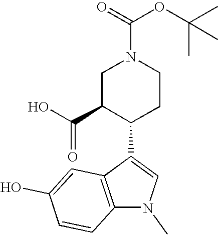

To a solution of Intermediate 31.5 (550 mg, 1.28 mmol) in MeOH is added Mg (583 mg, 24 mmol) at room temperature. After stirring for 19 h at room temperature, the reaction mixture is diluted with 5% aqueous KHSO₄ and extracted with Et₂O. The organic layer is washed H₂O with brine, dried over Na₂SO₄. Concentration under reduced pressure gives the crude material.

A mixture of the above crude material and 12% NaOMe in MeOH (10 mL) is stirred under reflux. After stirring for 3 h, 1N aqueous NaOH is added to the reaction mixture. After stirring at 80° C., the reaction mixture is acidified by 5% aqueous KHSO₄ and extracted with EtOAc. The organic layer is washed with H₂O with brine, dried over Na₂SO₄. Concentration under reduced pressure affords Intermediate 31.4 as a white amorphous; ES-MS: [M-^{t}Bu+H]⁺=319; HPLC: _{A}t_{Ret}=3.25 min.

### Intermediate 31.5

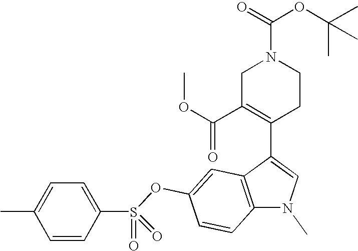

To a solution of Intermediate 31.6 (3.2 g, 7.5 mmol) and 4-trifluoromethanesulfonyloxy-5,6-dihydro-2H-pyridine-1,3-dicarboxylic acid 1-tert-butyl ester 3-methyl ester (3.5 g, 9 mmol, WO2004002957) in dioxane (38 mL) and H₂O (4 mL) are added K₃PO₄ (1.9 g, 9 mmol) and Pd(PPh₃)₄ (0.81 g, 0.7 mmol) under N₂ atmosphere, then the mixture is stirred at 60° C. After stirring for 1.5 h, the reaction is quenched by addition of H₂O and the resulting mixture is extracted with EtOAc. The organic layer is washed with H₂O and brine, and dried over MgSO₄ and concentrated under reducing pressure. The residue is purified by SiO₂ chromatography to afford Intermediate 31.5 as a brown amorphous material; ES-MS: [M+HtBu]⁺=485; HPLC: t_{Ret}=4.75 min.

### Intermediate 31.6

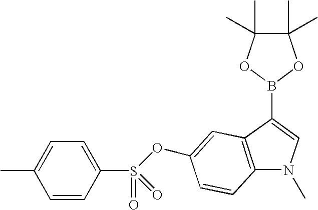

A mixture of Intermediate 31.7 (2 g, 6.6 mmol) and pyridiniumin tribromide (2.5 g, 9 mmol) in CH₂Cl₂ is stirred at 0° C. After stirring for 1 h, the mixture is diluted with CH₂Cl₂, and the organic layer is successively washed with aqueous Na₂SO₃ twice, H₂O and brine, then dried over Na₂SO₄. Concentration under reduced pressure affords the crude material.

To a solution of the above crude material in THF (100 mL) is added n-butyllithium (1.6 M in n-hexane, 4.7 mL, 7.6 mmol) at −78° C. After stirring for 0.5 h at −78° C., 2-Isopropoxy 4,4,5,5-tetramethyl-[1,3,2]-dioxaborolane (1.47 g, 7.9 mmol) is added to the mixture. After stirring at for 1 h at −78° C., the reaction mixture is quenched by 5% aqueous KHSO₄ and extracted with Et₂O. The organic layer is washed with H₂O with brine, dried over Na₂SO₄. Concentration under reduced pressure affords Intermediate 31.6 as colorless oil; ES-MS: [M+H]⁺=428; HPLC: _{A}t_{Ret}=4.82 min.

### Intermediate 31.7

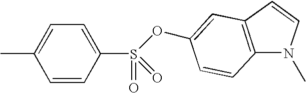

To a solution of 5-hydroxylindole (4 g, 35.4 mmol) in DMF (300 mL) is added NaH 60% dispersion in mineral oil (3.5 g, 88 mmol) at 0° C., then the mixture is stirred at 0° C. After stirring for 1 h at 0° C., tosyl chloride (17 g, 88.5 mmol) is added to the mixture, then the mixture is stirred at 0° C. After stirring for 1 h at 0° C., the reaction is quenched by 5% aqueous KHSO₄ to give purple solid, which is collected by filtration.

To a solution of the above solid in THF (200 mL) is added NaOMe in MeOH (25% w/w, 10 mL) at 0° C., then the mixture is stirred at 0° C. After stirring for 1 h at 0° C., the reaction mixture is quenched by 5% aqueous KHSO₄ and extracted with Et₂O. The organic layer is washed with H₂O with brine, dried over Na₂SO₄. Concentration under reduced pressure gives the crude material.

To a solution of the crude in DMF (100 mL) is added NaH 60% dispersion in mineral oil (1.4 g, 35 mmol), and the mixture is stirred at room temperature. After stirring for 1 h at room temperature, iodomethane (5 g, 35 mmol) is added to the mixture at 0° C., then the mixture is stirred at 0° C. for 1 h. The reaction mixture is quenched by 5% aqueous KHSO₄. The resulting solid material is collected by filtration then washed with Et₂O to give Intermediate 31.7 as a brown solid material; ES-MS: [M+H]⁺=302; HPLC: _{A}t_{Ret}=4.20 min.

### Intermediate 32.1

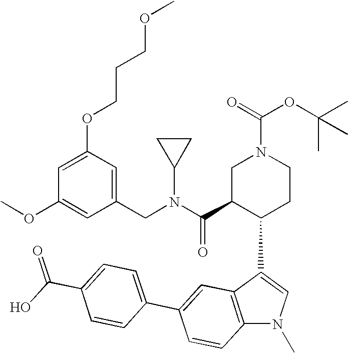

Intermediate 32.1 is synthesized by hydrolysis of Intermediate 32.2 analogously to the preparation of Intermediate 31.1. Yellow amorphous materials; ES-MS: [M+H]⁺=726; HPLC: t_{Ret}=4.59 min.

### Intermediate 32.2

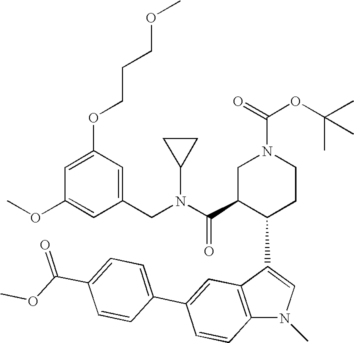

To a solution of Intermediate 32.3 (160 mg, 0.21 mmol) and 4-methylcarbonylphenylboronic acid (61 mg, 0.34 mmol) in dioxane (1 mL) and H₂O (0.2 mL) are added K₃PO₄ (72 mg, 0.34 mmol) and Pd(PPh₃)₄ (23 mg, 0.02 mmol) under N₂ atmosphere, then the mixture is stirred at 60° C. After stirring for 3 h, the reaction mixture is cooled down to room temperature, quenched by H₂O and extracted with Et₂O. The organic layer is washed with H₂O and brine, and dried over MgSO₄ and concentrated under reduced pressure. RP-HPLC purification affords Intermediate 32.2 as a yellow amorphous material; ES-MS: [M+H]⁺=740; HPLC: t_{Ret}=5.37 min.

### Intermediate 32.3

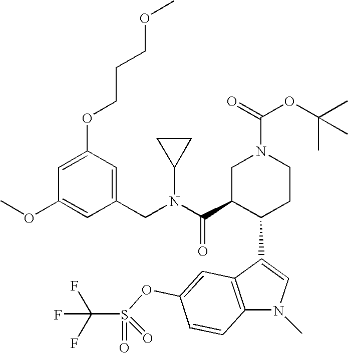

To a solution of Intermediate 31.3 (140 mg, 0.225 mmol) in CH₂Cl₂ (1 mL) are added diisopropylethylamine (170 mg, 1.35 mmol) and Tf₂O (96 mg, 0.34 mmol) at 0° C., then the mixture is stirred at 0° C. After stirring for 1 h at 0° C., the reaction mixture is quenched by H₂O and extracted with Et₂O. The organic layer is washed with 5% aqueous KHSO₄, H₂O and brine, and dried over MgSO₄ and concentrated under reduced pressure to give Intermediate 32.3 as a yellow oil material; ES-MS: [M+H]⁺=754; HPLC: t_{Ret}=5.32 min.

### Intermediate 33.1

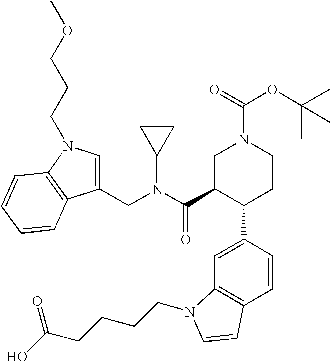

To a solution of Intermediate 33.2 (59.5 mg, 0.085 mmol) in MeOH (2 mL) is added 1N aqueous NaOH (0.5 mL) and stirred at 60° C. for 2 h. After cooling to room temperature and evaporated in vacuo, the resulting mixture is acidified with citric acid solution and extracted with Et₂O. The organic layer is dried over Na₂SO₄, and concentration under reduced pressure gives Intermediate 33.1, which is directly used without any purification.

### Intermediate 33.2

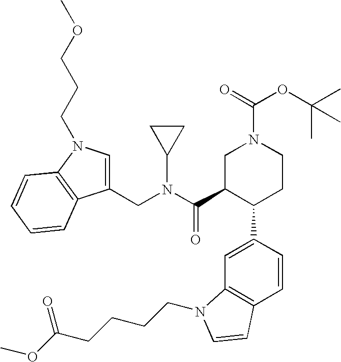

A mixture of Intermediate 33.3 (102.1 mg, 0.14 mmol), TBAF (112.0 mg, 0.43 mmol) and ethylenediamine (61 μL, 0.91 mmol) in DMF (2 mL) is stirred under N₂ at 50° C. for 7 h then 70° C. for 1 h. After adding H₂O, the reaction mixture is extracted with EtOAc. The combined organic phases are washed with H₂O, dried (Na₂SO₄), concentrated. A mixture of the residue, methyl 5-bromovalerate (30 μL, 0.21 mmol) and NaH (8.4 mg, 0.21 mmol) in DMF (2 mL) is stirred at RT for 3 h. After adding ice and H₂O, the reaction mixture is extracted with EtOAc. The combined organic phases are wash with brine and dried (Na₂SO₄). Concentration under reduced pressure and silica gel column chromatography affords Intermediate 33.2 as a yellow oil ES-MS: M+H=699; HPLC: t_{Ret}=5.29 min.

### Intermediate 33.3

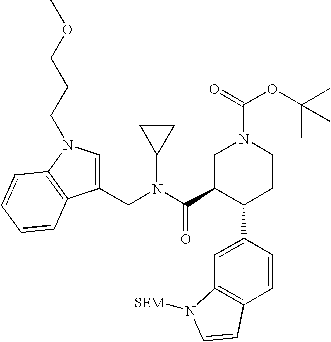

Intermediate 33.3 is synthesized by condensation of Intermediate 33.4 (503.5 mg, 1.06 mmol) and Intermediate 1.2 (328.9 mg, 1.27 mmol) analogously to the preparation of Intermediate 1.1. White amorphous material; ES-MS: M⁺=715; HPLC: t_{Ret}=5.92 min.

### Intermediate 33.4

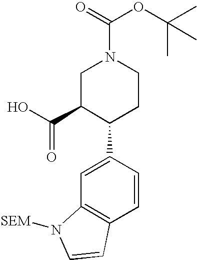

Intermediate 33.4 is synthesized by 1,4-reduction, epimerization and hydrolysis of Intermediate 33.5 (2.72 g, 5.59 mmol) analogously to the preparation of Intermediate 1.4 and 1.5. Colorless oil; ES-MS: M+H=475; HPLC: t_{Ret}=4.93 min.

### Intermediate 33.5

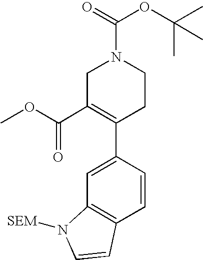

Intermediate 33.5 is synthesized by condensation of 4-trifluoromethanesulfonyloxy-5,6-dihydro-2H-pyridine-1,3-dicarboxylic acid 1-tert-butyl ester 3-methyl ester (3.30 g, 8.49 mmol) and Intermediate 33.6 (3.17 g, 8.49 mmol) analogously to the preparation of Intermediate 1.6. Colorless solid; ES-MS: M+H=487; HPLC: t_{Ret}=5.50 min.

### Intermediate 33.6

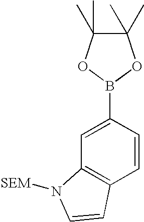

Intermediate 33.6 is synthesized by cross-coupling of Intermediate 33.7 (3.35 g, 10.20 mmol) analogously to the preparation of Intermediate 19.4. Light yellow solid; ES-MS: M+H=374; HPLC: t_{Ret}=5.67 min.

### Intermediate 33.7

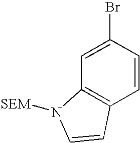

To a mixture of 6-bromoindole (2 g, 10.20 mmol) and NaH (448.9 mg, 11.22 mmol) in DMF (40 mL) is added SEMCl (1.99 mL, 4.3 mmol) dropwise at 0° C. After stirring for 1 h, the reaction mixture is quenched with ice, and the mixture is extracted with Et₂O. The combined organic phases are washed with brine and dried (Na₂SO₄). Concentration under reduced pressure and silica gel flash chromatography give Intermediate 33.7 as colorless oil; ES-MS: M+H=326; HPLC: t_{Ret}=5.58 min.

### Intermediate 34.1

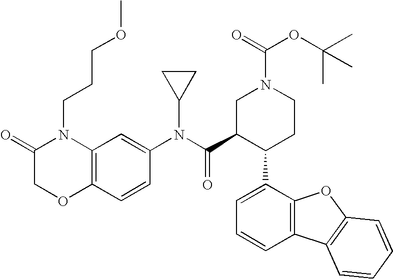

Intermediate 34.1 is synthesized by condensation of Intermediate 2.2 (151.1 mg, 0.38 mmol) and Intermediate 27.2 (105.6 mg, 0.38 mmol) analogously to the preparation of Intermediate 27.1. White amorphous material; ES-MS: M+H=654; HPLC: t_{Ret}=4.70 min.

### Intermediate 35.1

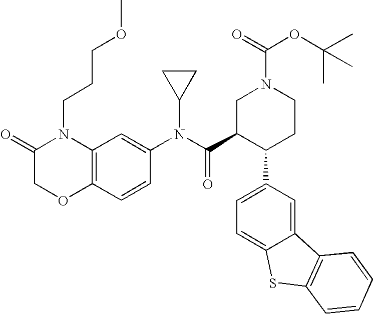

Intermediate 35.1 is synthesized by condensation of Intermediate 12.2 (207.3 mg, 0.50 mmol) and Intermediate 27.2 (139.2 mg, 0.5 mmol) analogously to the preparation of Intermediate 27.1. White amorphous material; ES-MS: M+H=670; HPLC: t_{Ret}=4.91 min.

### Intermediate 36.1

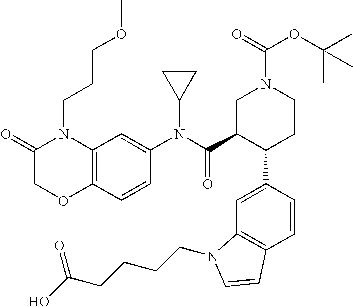

Intermediate 36.1 is synthesized by hydrolysis of Intermediate 36.2 (83.5 mg, 0.12 mmol) analogously to the preparation of Intermediate 33.1. White amorphous material; ES-MS: M+H=703; HPLC: t_{Ret}=4.05 min.

### Intermediate 36.2

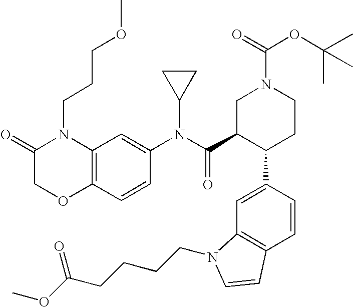

Intermediate 36.2 is synthesized by deprotection and alkylation of Intermediate 36.3 (320.1 mg, 0.44 mmol) analogously to the preparation of Intermediate 33.2. White amorphous material; ES-MS: M+H=717; HPLC: t_{Ret}=4.55 min.

### Intermediate 36.3

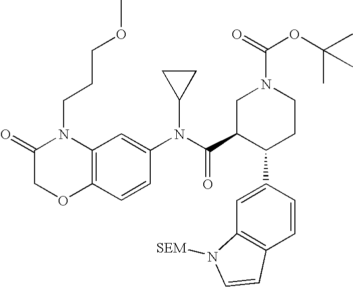

Intermediate 36.3 is synthesized by condensation of Intermediate 33.4 (553.1 mg, 1.17 mmol) and Intermediate 27.2 (322 mg, 1.17 mmol) analogously to the preparation of Intermediate 27.1. White amorphous material; ES-MS: M=733; HPLC: t_{Ret}=5.39 min.

### Intermediate 37.1

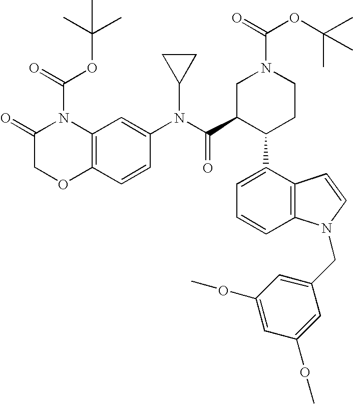

Intermediate 37.1 is synthesized by condensation of Intermediate 19.2 (200.1 mg, 0.40 mmol) and Intermediate 37.2 (123.1 mg, 0.40 mmol) analogously to the preparation of Intermediate 27.1. White amorphous material; ES-MS: M+H=781; HPLC: t_{Ret}=5.03 min.

### Intermediate 37.2

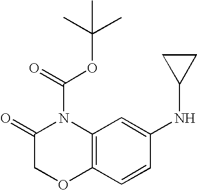

To a solution of Intermediate 27.3 (200 mg, 1.0 mmol) and in THF (5 mL), Boc₂O (240 mg, 1.10 mmol) and DMAP (134 mg, 1.10 mmol) are added under N₂ at 0° C. After stirring RT for 16 h, H₂O is added and the reaction mixture is extracted with EtOAc. The combined organic phases are washed with H₂O, and dried (Na₂SO₄). Concentration under reduced pressure and silica gel flash chromatography give Intermediate 37.2 as colorless amorphous; ES-MS: M+H=305; HPLC: _{A}t_{Ret}=3.77 min.

### Intermediate 38.1

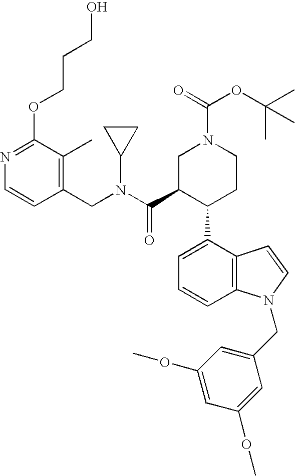

A mixture of Intermediate 38.2 (126.3 mg, 0.15 mmol) and TBAF in THF (0.38 μL, 0.38 mmol) are stirred under N₂ at RT for 1 h. After adding H₂O, the reaction mixture is extracted with EtOAc. The combined organic phases are washed with H₂O, dried (Na₂SO₄), and concentration under reduced pressure gives Intermediate 38.2 as White amorphous material; ES-MS: M+H=713; HPLC: t_{Ret}=4.14 min.

### Intermediate 38.2

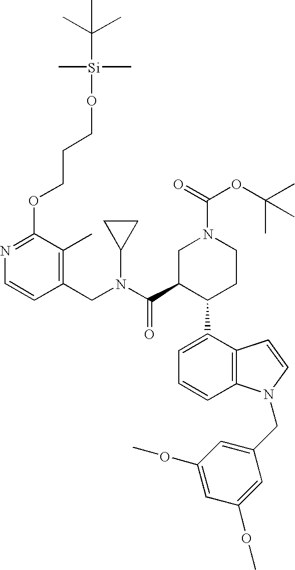

Intermediate 38.2 is synthesized by condensation of Intermediate 19.2 (134.0 mg, 0.27 mmol) and {2-[3-tert-Butyldimethylsilanyloxy]propoxy}-3-methylpyridin-4-ylmethyl}-cyclopropylamine (95.6 mg, 0.27 mmol) (see e.g. WO 2005/054244) analogously to the preparation of Intermediate 1.1. White amorphous material; ES-MS: M+H=827; HPLC: t_{Ret}=6.12 min.

### Intermediate 39.1

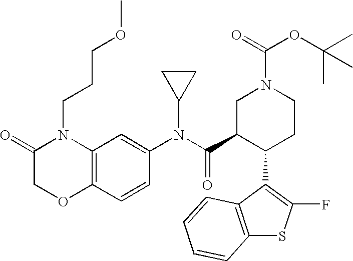

Intermediate 39.1 is synthesized by coupling reaction of Intermediate 39.2 (103.2 mg, 0.27 mmol) and Intermediate 27.2 (90.08 mg, 0.326 mmol) analogously to the preparation of Intermediate 27.1. White solid; ES-MS: M+H=638; HPLC: t_{Ret}=4.55 min.

### Intermediate 39.2

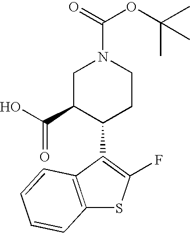

Intermediate 39.2 is synthesized by 1,4-reduction, epimerization and hydrolysis of Intermediate 39.3 (213.1 mg, 0.544 mmol) analogously to the preparation of Intermediate 1.5 and 1.4. White solid; ES-MS: M-^{t}Bu+H=324; HPLC: t_{Ret}=3.95 min.

### Intermediate 39.3

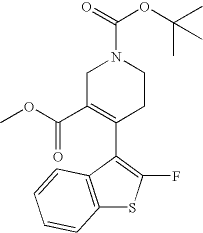

Intermediate 39.3 is synthesized by coupling reaction of 4-trifluoromethanesulfonyloxy-5,6-dihydro-2H-pyridine-1,3-dicaroxylic acid 1-tert-butyl ester 3-methyl ester (801.5 mg, 2.058 mmol) and Intermediate 39.4 (484.2 mg, 2.47 mmol) analogously to the preparation of Intermediate 1.6. white solid; ES-MS: M-^{t}Bu+H=336; HPLC: _{A}t_{Ret}=4.57 min.

### Intermediate 39.4:

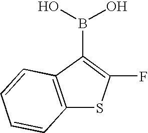

To the solution of Intermediate 39.5 (93.5 mg, 0.61 mmol) in THF (5 mL) under N₂ at −78° C., n-BuLi (0.57 ml, 1.60 M in hexane) is added. After stirring at that temperature for 1 hr, trimethoxyboronic ester (0.108 mL, 0.915 mmol) is added. The reaction mixture is warmed to RT and stirred at that temperature for a few minutes. Then, 1N HCl aq. is added and stirred at RT for 5 min. The resulting solution is extracted with EtOAc and organic phase is wshed with brine, dried over Na₂SO₄, concentrated under reduced pressure and subjected to silica gel chromatography to give the intermediate 39.4 as sirup. Rf=0.38 (hexane/EtOAC=1/1) HPLC: _{A}t_{Ret}=2.82 min.

### Intermediate 39.5

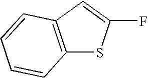

To the solution of benzothiophene (2.67 g, 0.0198 mol) in THF (200 mL) under N₂ at −78° C., n-BuLi (18.6 ml, 1.60 M in hexane) is added. After stirring at that temperature for 1 hr, N-fluorobenzenesulofoneimide (12.48 g, 0.0396 mol) is added. The reaction mixture is warmed to RT and stirred at that temperature for a few minutes. Then, sat. NH₄Cl aq. is added. The resulting solution is extracted with EtOAc and organic phase is dried over Na₂SO₄, concentrated under reduced pressure and subjected to silica gel chromatography to give the intermediate TAI097.5 as sirup. Rf=0.77 (hexane/EtOAC=10/1) HPLC: _{A}t_{Ret}=4.02 min. Ref. Tetrahedron Lett. 1977, 2797-2800.

### Intermediate 40.1

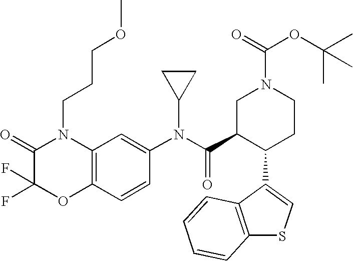

Intermediate 40.1 is synthesized by coupling of Intermediate 8.2 (200 mg, 0.55 mmol) and Intermediate 40.2 (189.8 mg, 0.608 mmol) analogously to the preparation of Intermediate 27.1. White solid; ES-MS: M+H=656; HPLC: t_{Ret}=4.82 min.

### Intermediate 40.2

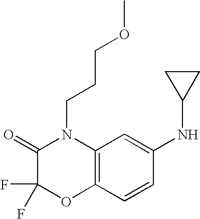

Intermediate 40.2 is synthesized by N-alkylation of intermediate 40.3 (2.38 g, 9.9 mmol) analogously to the preparation of intermediate 27.2. White crystal; ES-MS: M+H=313; HPLC: _{C}t_{Ret}=1.94 min.

### Intermediate 40.3

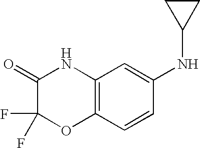

Intermediate 40.4 (140 mg, 0.42 mmol) and potassium carbonate (210 mg, 2.1 mmol) in MeOH (1.4 mL) is stirred at r.t. for 1.5 h. After adding water, the reaction mixture is extracted with EtOAc. The combined organic phases are washed with H₂O and dried (MgSO₄). Concentration under reduced pressure and recrystallization give Intermediate 40.3. White crystal; ES-MS: M+H=241: _{C}t_{Ret}=1.73 min.

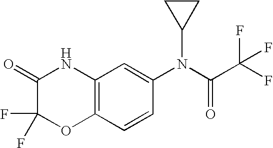

### Intermediate 40.4

Intermediate 40.5 (1.7 g, 4.08 mmol) and potassium carbonate (843 mg, 6.11 mmol) in DMF (25 mL) is stirred at 70° C. for 5 h. The reaction mixture is concentrated under reduced pressure. The evaporated residue is diluted with EtOAc, washed with H₂O and dried (MgSO₄). Concentration under reduced pressure and recrytallization give Intermediate 40.4: white powder; ES-MS: M+H=337: _{C}t_{Ret}=1.81 min.

### Intermediate 40.5

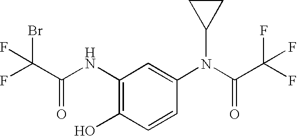

Intermediate 40.6 (1.25 g, 4.8 mmol), potassium carbonate (1.66 g, 12 mmol) and bromo difluoroacetyl chloride (1.02 g, 5.28 mmol) in THF (15 mL) is stirred at 0° C. for 30 min. After adding KHSO₄aq., the reaction mixture is extracted with EtOAc. The combined organic phases are washed with sat.NaHCO₃aq. and dried (MgSO₄). Concentration under reduced pressure and silica gel flash chromatography give Intermediate 40.5: yellow crystal; ES-MS: M+H=417: _{C}t_{Ret}=1.82 min.

### Intermediate 40.6

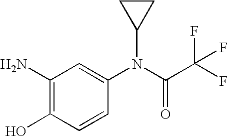

To a solution of Intermediate 40.7 (13 g, 44.8 mmol) in EtOH (60 mL) is added NH₄Cl (4.8 g, 89.6 mmol), water (60 mL) and Zn (14.6 g, 224 mmol). The resulting mixture is stirred at 80° C. for 1 h. The reaction mixture is filtered via celite pad and the celite cake is washed with EtOH and EtOAc. Concentration under reduced pressure and silica gel flash chromatography gives Intermediate 40.6: red crystal; ES-MS: M+H=261: _{C}t_{Ret}=1.31 min.

### Intermediate 40.7

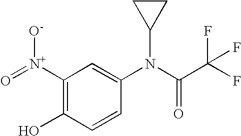

To a solution of Intermediate 40.8 (19 g, 82.4 mmol) and pyridine (32.6 g, 412 mmol) in dichloromethane (200 mL) is added trifluoroacetic anhydride (25 g, 247 mmol) at 0° C. and stirred for 30 min. After adding 2M HClaq (83 mL), the reaction mixture is extracted with dichloromethane. The combined organic phases are washed with water, sat.NaHCO₃aq. and dried (MgSO₄). Concentration under reduced pressure and recrystallization give Intermediate 40.7: yellow crystal; ES-MS: M+H=291: _{C}t_{Ret}=1.82 min.

### Intermediate 40.8

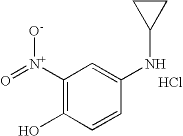

Intermediate 40.9 (23 g, 92 mmol) in EtOAc (50 mL) is added to 4M HCl in EtOAc with small amount of water at r.t. The resulting mixture is stirred for 15 min. The resulting yellow precipitate is collected by filtration and the solid is washed with EtOAc to give Intermediate 40.8: yellow powder; ES-MS: M+H−HCl=195: _{C}t_{Ret}=1.78 min.

### Intermediate 40.9

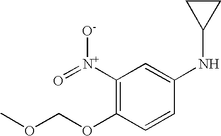

Intermediate 40.9 is synthesized by reduction of intermediate 40.10 (29 g, 130 mmol) analogously to the preparation of intermediate 27.5. Red oil; ES-MS: M+H=239; HPLC: _{C}t_{Ret}=1.83 min.

### Intermediate 40.10

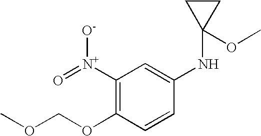

To a solution of Intermediate of 27.5 (29.1 g, 130 mmol) in dichloromethane (300 mL) is added N,N-ethyldiisopropylamine (67 g, 520 mmol) and MOMCl (10.5 g, 130 mmol) at 0° C. The reaction mixture is stirred for overnight. After being neutralized by 1M HClaq., the reaction mixture is extracted with dichlomethane. The combined organic phases are washed with sat.NaHCO₃aq. and dried (MgSO₄). Concentration under reduced pressure gives Intermediate 40.10: red oil; ES-MS: M+H=_{C}t_{Ret}=1.82 min.

### Intermediate 41.1

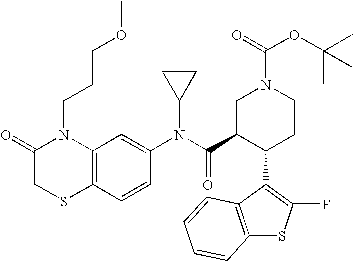

Intermediate 41.1 is synthesized by coupling of intermediate 39.2 (151.0 mg, 0.398 mmol) and Intermediate 41.2 (116 mg, 0.398 mmol) analogously to the preparation of Intermediate 27.1. White solid; ES-MS: M+H=654; HPLC: t_{Ret}=4.72 min.

### Intermediate 41.2

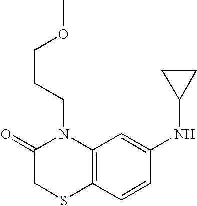

Intermediate 41.2 is synthesized by alkylation of Intermediate 41.3 (228 mg, 1.0 mmol) made analogously to a known method (see e.g. European Journal of Medicinal Chemistry 1998, 33, 957-967. or EP 432893). Orange solid; ES-MS: M+H=293; HPLC: _{A}t_{Ret}=3.70 min.

### Intermediate 41.3

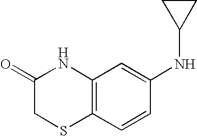

Intermediate 41.3 is synthesized by reduction of Intermediate 41.4 (143 g, 0.5 mmol) analogously to the preparation of Intermediate 27.3. Brown solid; ES-MS: M+H=221; HPLC: _{A}t_{Ret}=2.87 min.

### Intermediate 41.4

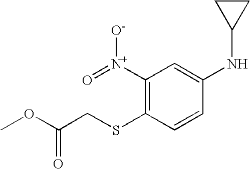

A mixture of Intermediate 41.5 (4.73 g, 2.4 mmol), methylthioglycolate (237 μL, 2.65 mmol), and NaH (115 mg, 2.88 mmol) in DMF (10 mL) is stirred under N₂ at 0° C. for 1 h. After adding H₂O, the reaction mixture is extracted with EtOAc. The combined organic phases are washed with H₂O and dried (Na₂SO₄). Concentration under reduced pressure and silica gel flash chromatography give Intermediate 41.4 as orange oil; ES-MS: M+H=283; HPLC: _{A}t_{Ret}=3.84 min.

### Intermediate 41.5

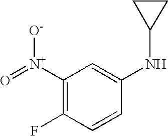

Intermediate 41.5 is synthesized by cyclopropanation of 4-fluoro-3-nitroaniline (3.25 g, 40.0 mmol) analogously to the preparation of intermediate 27.5. Orange oil; ES-MS: M+H=197; HPLC: _{A}t_{Ret}=3.82 min.

### Intermediate 42.1

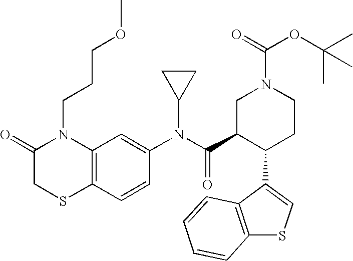

Intermediate 42.1 is synthesized by coupling of Intermediate 8.2 (200 mg, 0.55 mmol) and Intermediate 41.2 (160.8 mg, 0.55 mmol) analogously to the preparation of Intermediate 27.1. White solid; ES-MS: M+H=636; HPLC: t_{Ret}=4.52 min.

### Intermediate 43.1

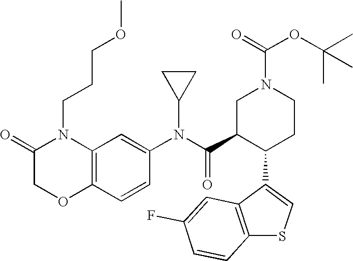

Intermediate 43.1 is synthesized by condensation of Intermediate 43.2 (166.4 mg, 0.44 mmol) and Intermediate 27.2 (133.3 mg, 0.48 mmol) analogously to the preparation of Intermediate 27.1. White amorphous material; ES-MS: M+H=638; HPLC: t_{Ret}=4.39 min.

### Intermediate 43.2

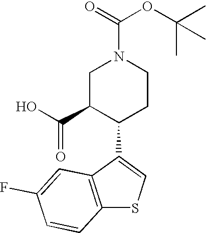

Intermediate 43.2 is synthesized by 1,4-reduction (Mg-MeOH), oxidation (MnO₂), epimerization (NaOMe) and hydrolysis of Intermediate 43.3 (206.1 g, 0.53 mmol) analogously to the preparation of Intermediate 1.4, 1.5 and 9.2. Colorless oil; ES-MS: M-^{t}Bu=324; HPLC: t_{Ret}=3.84 min.

### Intermediate 43.4

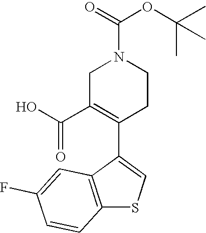

To a solution of 5-fluoro-benzo[b]thiophen-3(2H)-one (1.41 g, 7.81 mmol) (see e.g. WO 2004/099191) in dichloromethane (40 ml) is added N,N-diisopropylethylamine (3.3 mL, 19.53 mmol) and trifluoromethanesulfonyl anhydride (1.3 mL, 9.37 mmol) at −78° C. then stirred for 1.5 h. After adding water, the reaction mixture is extracted with dichloromethane. The combined organic phases are concentrated under reduced pressure gives the corresponding triflate.

Intermediate 43.4 is synthesized by condensation of intermediate 43.5 (2.64 g, 7.19 mmol) and the above triflate analogously to the preparation of Intermediate 1.6. Orange amorphous material; ES-MS: M-^{t}Bu=336; HPLC: t_{Ret}=4.38 min.

### Intermediate 43.5

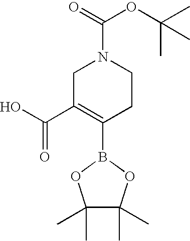

Intermediate 43.5 is synthesized by cross-coupling of 4-trifluoromethanesulfonyloxy-5,6-dihydro-2H-pyridine-1,3-dicarboxylic acid 1-tert-butyl ester 3-methyl ester (2.13 g, 5.74 mmol) analogously to the preparation of Intermediate 19.4. Light yellow solid; ES-MS: M-^{t}Bu=312; HPLC: t_{Ret}=4.03 min.

### Intermediate 44.1 & 45.1

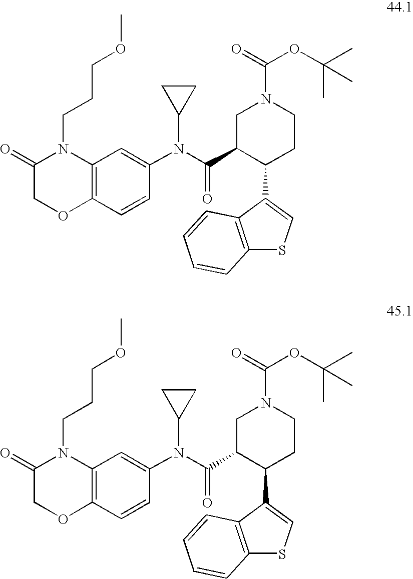

The two enantiomers of racemic intermediate 27.1 are separated by chiral, separative HPLC (HPLC, Chiralcel OD-H, n-hexane/EtOH/MeOH 90/5/5) and afford intermediate 44.1 (eut) as a white amorphous material: t_{R} (HPLC, Chiral OD, 20 um, 0.46×25 cm, n-hexane/EtOH/MeOH 90/5/5) 12.032 min (peak 1). Intermediate 45.1 (dist) is also isolated as a white amorphous material: t_{R} (HPLC, Chiral OD, 20 um, 0.46×25 cm, n-hexane/EtOH/MeOH 90/5/5) 14.741 min (peak 2).

### Example 46

### Soft Capsules

5000 soft gelatin capsules, each comprising as active ingredient 0.05 g of any one of the compounds of formula I mentioned in any one of the preceding Examples, are prepared as follows:

1. Composition

| | | |
|---|---|---|
| Active ingredient | 250 | g |
| Lauroglycol | 2 | liters |

Preparation process: The pulverized active ingredient is suspended in Lauroglykol® (propylene glycol laurate, Gattefosse S. A., Saint Priest, France) and ground in a wet pulverizer to produce a particle size of about 1 to 3 μm. 0.419 g portions of the mixture are then introduced into soft gelatin capsules using a capsule-filling machine.

### Example 32

### Tablets Comprising Compounds of the Formula I

Tablets, comprising, as active ingredient, 100 mg of any one of the compounds of formula I in any one of the preceding Examples are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinylpolypyrrolidone, cross-linked (BASF, Germany). Aerosil® is silicon dioxide (Degussa, Germany).

1. A compound of the formula I, 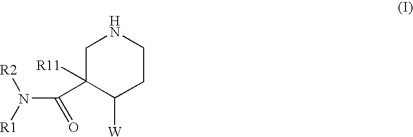
wherein
R1 is hydrogen, unsubstituted or substituted alkyl, unsubstituted or substituted alkenyl, unsubstituted or substituted alkynyl, unsubstituted or substituted aryl, unsubstituted or substituted heterocyclyl or unsubstituted or substituted cycloalkyl;
R2 is unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
W is unsubstituted or substituted polycyclic heterocyclyl or unsubstituted or substituted polycyclic aryl; and
R11 is hydrogen, hydroxy, halo, C₁-C₇-alkyl, halo-C₁-C₇-alkyl, cycloalkyl, halo-substituted cycloalkyl, C₁-C₇-alkoxy, halo-C₁-C₇-alkoxy or cyano,
or a salt thereof.

2. A compound of the formula I according to claim 1, wherein
R1 is hydrogen, alkyl, cycloalkyl substituted alkyl or cycloalkyl;
R2 is substituted alkyl where the substituents are selected from unsubstituted or substituted aryl and unsubstituted or substituted heterocyclyl, or is unsubstituted or substituted heterocyclyl;
W is unsubstituted or substituted polycyclic heterocyclyl or unsubstituted or substituted polycyclic aryl; and
R11 is hydrogen;
or a pharmaceutically acceptable salt thereof.

3. (canceled)

4. A compound of the formula I according to claim 1, wherein
R1 is hydrogen, C₁-C₇-alkyl, C₃-C₈-cycloalkyl or C₃-C₈-cycloalkyl-C₁-C₇-alkyl;
R2 is phenyl, phenyl-C₁-C₇-alkyl, indolyl, indolyl-C₁-C₇-alkyl, 2H-1,4-benzoxazin-3(4H)-onyl, 2H-1,4-benzoxazin-3(4H)-onyl-C₁-C₇-alkyl, where each phenyl, indolyl or 2H-1,4-benzoxazin-3(4H)-onyl is unsubstituted or substituted by one or more moieties independently selected from C₁-C₇-alkyl, C₁-C₇-alkoxy-C₁-C₇-alkyl, C₁-C₇-alkoxy-C₁-C₇-alkoxy-C₁-C₇-alkyl, halo, C₁-C₇-alkoxy and C₁-C₇-alkoxy-C₁-C₇-alkoxy;
W is naphthyl, indolyl, benzofuranyl, benzothienyl, quinolyl, isoquinolyl, carbazolyl, 9-thiafluorenyl or 9-oxafluorenyl, each of which is unsubstituted or substituted by one or more moieties independently selected from C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano;
and R11 is hydrogen;
or a pharmaceutically acceptable salt thereof.

5. A compound of the formula I according to claim 1, wherein
R1 is hydrogen, C₁-C₇-alkyl, C₃-C₈-cycloalkyl or C₃-C₈-cycloalkyl-C₁-C₇-alkyl;
R2 is phenyl, phenyl-C₁-C₇-alkyl, indolyl, indolyl-C₁-C₇-alkyl, 2H-1,4-benzoxazin-3(4H)-onyl, 2H-1,4-benzoxazin-3(4H)-onyl-C₁-C₇-alkyl, 4H-benzo[1,4]thiazin-3-onyl, 4H-benzo[1,4]thiazin-3-onyl-C₁-C₇-alkyl, pyridyl, and pyridyl-C₁-C₇-alkyl where each phenyl, indolyl, 2H-1,4-benzoxazin-3(4H)-onyl, 4H-benzol[1,4]thiazin-3-onyl, 4H-benzo[1,4]thiazin-3-only-C₁-C₇-alkyl, pyridyl, or pyridyl-C₁-C₇-alkyl is unsubstituted or substituted by one to three, moieties independently selected from C₁-C₇-alkyl, C₁-C₇-alkoxy-C₁-C₇-alkyl, C₁-C₇-alkoxyC₁-C₇-alkoxy-C₁-C₇-alkyl, halo, C₁-C₇-alkoxy, hydroxy-C₁-C₇-alkoxy and C₁-C₇-alkoxy-C₁-C₇-alkoxy;
W is indolyl, benzofuranyl, benzothienyl, quinolyl, isoquinolyl, carbazolyl, 9-thia-fluorenyl or 9-oxafluorenyl, each of which is unsubstituted or substituted by one to three moieties independently selected from C₁-C₇-alkyl, halo, carboxy-C₁-C₇-alkoxy, carboxy-C₁-C₇-alkyl and phenyl-C₁-C₇-alkyl or naphthyl-C₁-C₇-alkyl wherein the phenyl or naphthyl is unsubstituted or substituted by up to three moieties independently selected from C₁-C₇-alkyl, halo, carboxy, hydroxy, C₁-C₇-alkoxy, amino, N-mono- or N,N-di-(C₁-C₇-alkyl)-amino, C₁-C₇-alkoxycarbonyl, carbamoyl, sulfamoyl and cyano;
and R11 is hydrogen;
or a pharmaceutically acceptable salt thereof.

6. A compound of the formula I according to claim 1, having the configuration given in the following formula IA, 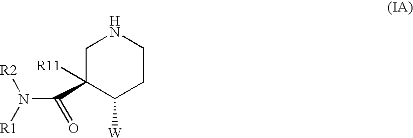
wherein R1, R2, R11 and W are as defined in claim 1;
or a pharmaceutically acceptable salt thereof.

7. A compound of the formula I according to claim 1, selected from the group of compounds having the formulae: 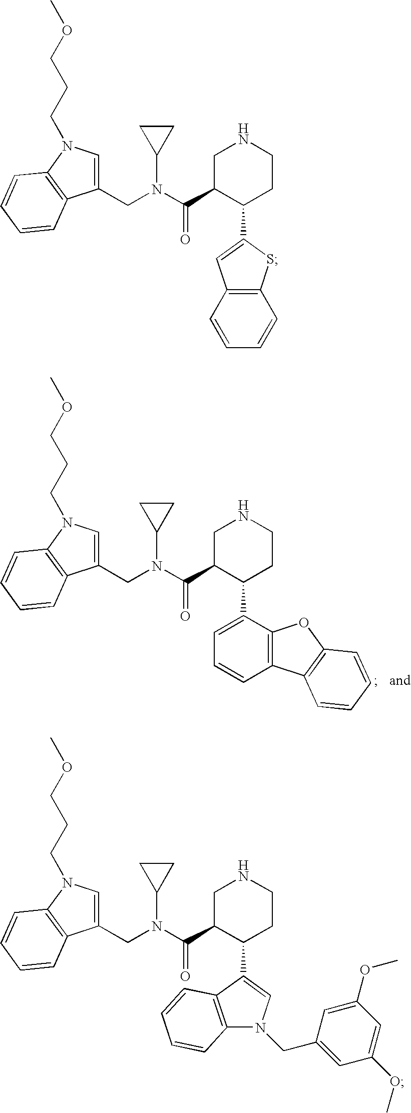
each of which in the presence of the compound of the respective mirror image can be present as enantiomeric mixture, e.g. as racemate, or preferably as pure enantiomer as shown in the formulae;
or a pharmaceutically acceptable salt thereof.

8. A compound of the formula I according to claim 1, selected from the group of compounds of the formula I represented by the following formula and the definition of its moieties in the following table:

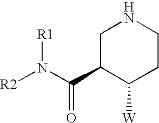

| Compound | R1 | R2 | W |
|---|---|---|---|
| 4 | 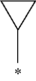 | 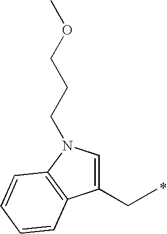 | 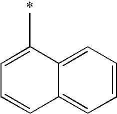 |
| 5 | 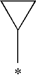 | 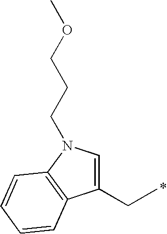 | 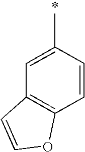 |
| 6 | 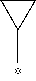 | 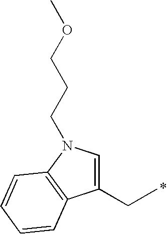 | 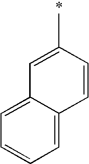 |
| 7 | 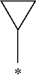 | 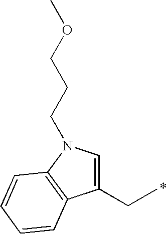 | 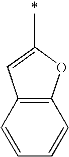 |
| 8 | 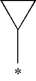 | 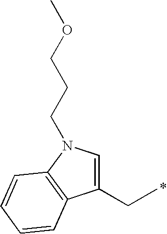 | 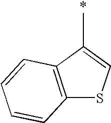 |
| 9 | 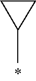 | 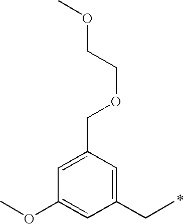 | 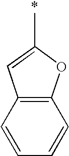 |
| 10 | 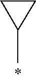 | 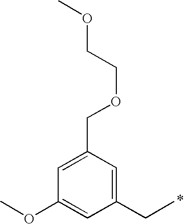 | 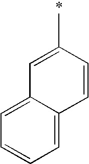 |
| 11 | 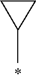 | 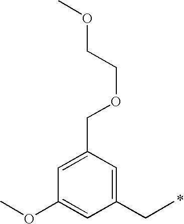 | 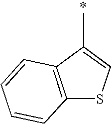 |
| 12 | 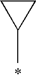 | 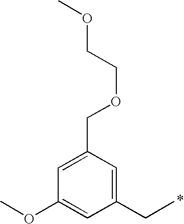 | 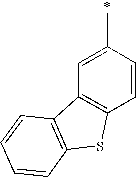 |
| 13 | 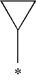 | 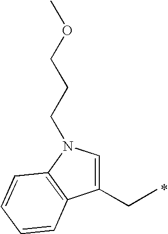 | 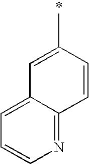 |
| 14 | 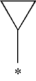 | 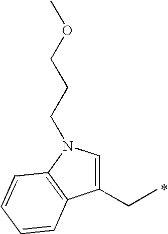 | 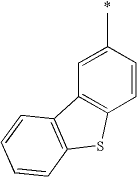 |
| 15 | 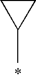 | 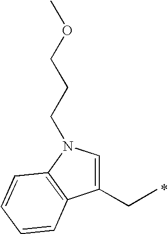 | 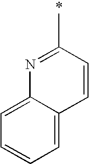 |
| 16 | 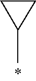 | 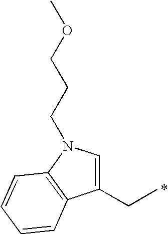 | 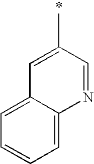 |
| 17 | 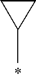 | 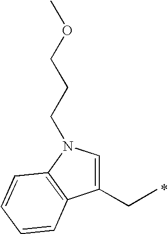 | 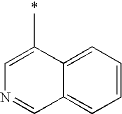 |
| 18 | 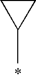 | 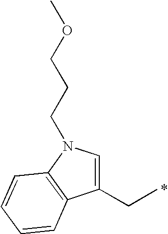 | 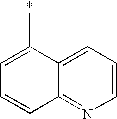 |
| 19 | 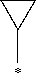 | 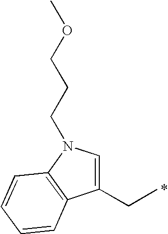 | 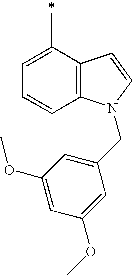 |
| 20 | 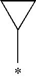 | 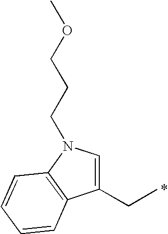 | 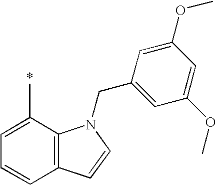 |
| 21 | 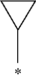 | 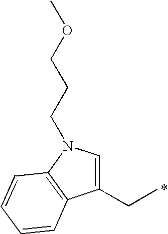 | 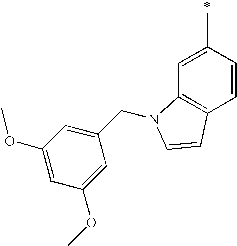 |
| 22 | 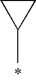 | 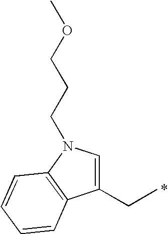 | 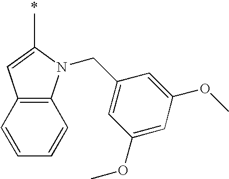 |
| 23 | 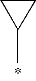 | 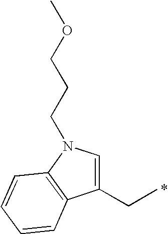 | 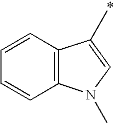 |
| 24 | 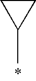 | 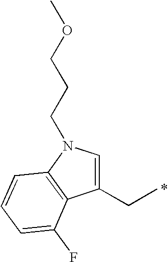 | 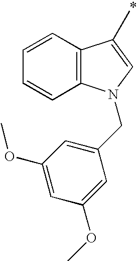 |
| 25 | 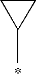 | 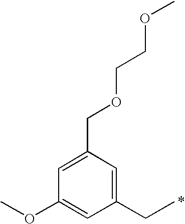 | 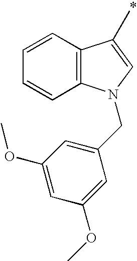 |
| 26 | 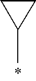 | 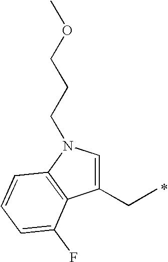 | 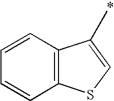 |
| 27 | 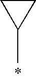 | 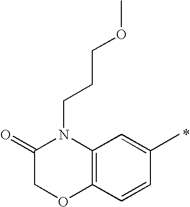 | 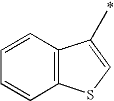 |
| 28 | 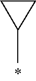 | 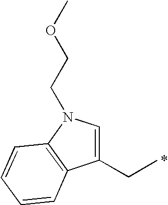 | 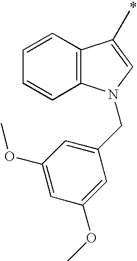 |
| 29 | 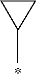 | 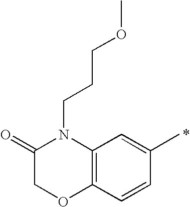 | 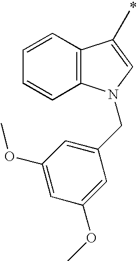 |
| 30 | 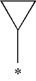 | 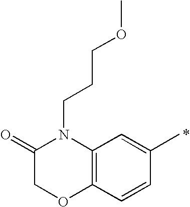 | 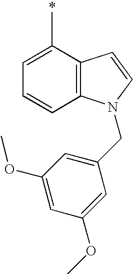 |
| 31 | 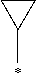 | 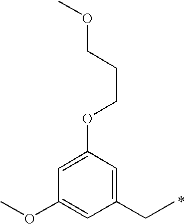 | 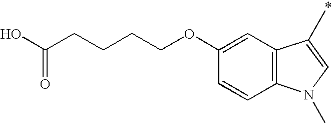 |
| 32 | 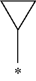 | 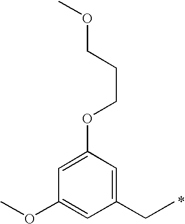 | 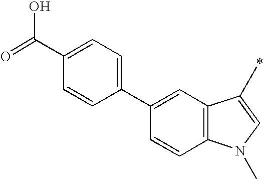 |
| 33 | 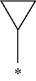 | 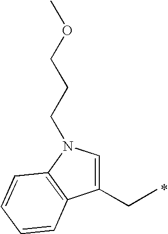 | 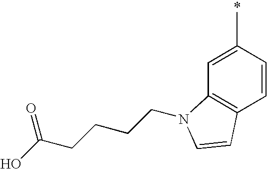 |
| 34 | 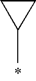 | 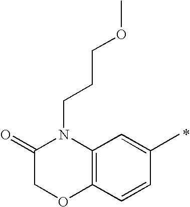 | 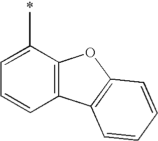 |
| 35 | 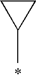 | 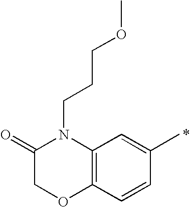 | 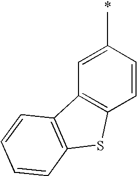 |
| 36 | 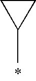 | 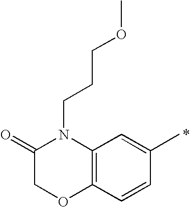 | 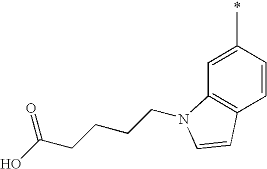 |
| 37 | 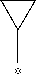 | 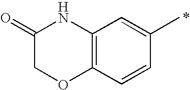 | 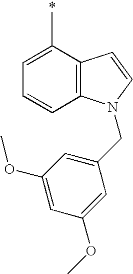 |
| 38 | 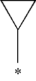 | 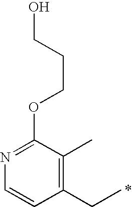 | 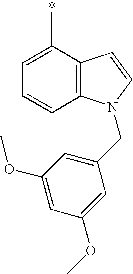 |
| 39 | 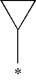 | 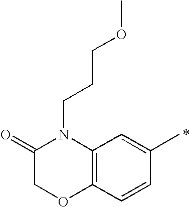 | 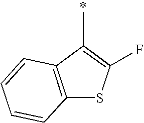 |
| 40 | 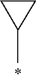 | 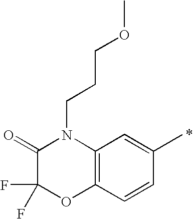 | 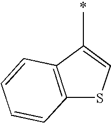 |
| 41 | 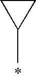 | 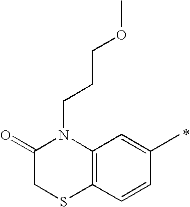 | 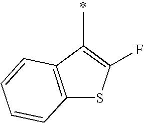 |
| 42 | 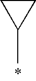 | 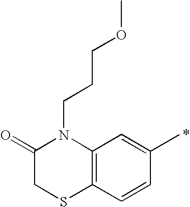 | 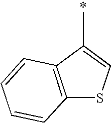 |
| 43 | 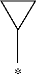 | 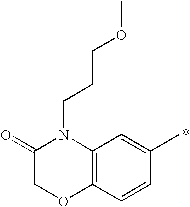 | 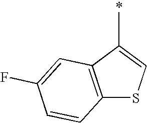 |
| 44 (eut) | 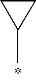 | 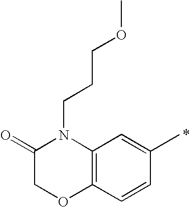 | 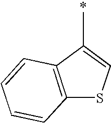 |
| 45 (dist) | 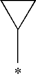 | 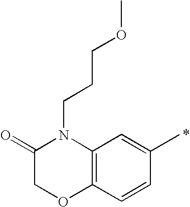 | 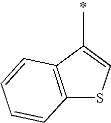 |

each of which in the presence of the compound of the respective mirror image can be present as enantiomeric mixture e.g. as racemate, or preferably as pure enantiomer as shown in the formulae;
or a pharmaceutically acceptable salt thereof.

9.-12. (canceled)

13. A pharmaceutical formulation, comprising a compound of the formula I, or a pharmaceutically acceptable salt thereof, according to claim 1 and at least one pharmaceutically acceptable carrier material.

14. A method of treatment a disease that depends on activity of renin, comprising administering to a warm-blooded animal in need of such treatment a pharmaceutically effective amount of a compound of the formula I, or a pharmaceutically acceptable salt thereof, according to claim 1.

15. A process for the manufacture of a compound of the formula I, or a pharmaceutically acceptable salt thereof, according to claim 1, said process comprising the step(s) of:
reacting a carbonic acid of the formula II, 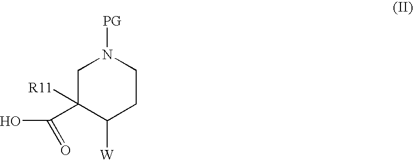
or a reactive derivative thereof, wherein PG is a protecting group and W and R11 are as defined for a compound of the formula I, with an amino compound of the formula III,
R1—NH—R2 (III)
wherein R1 and R2 are as defined for a compound of the formula I,
and, if desired, subsequent to this condensation reaction, converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials of the formula II and/or 111, in addition to specific protecting groups mentioned, further protecting groups may be present, and any protecting groups are removed at an appropriate stage (especially before or after a reaction mentioned under “if desired”) in order to obtain a corresponding compound of the formula I, or a salt thereof.
